(19) United States
(12) Patent Application Publication
(10) Pub. No.: US 2024/0270804 A1
(43) Pub. Date: Aug. 15, 2024

(54) PROMOTER-ENHANCER SEQUENCES OF THE HUMAN TROPONIN T GENE FOR SELECTIVE EXPRESSION IN CARDIAC MYOCYTES

(71) Applicant: Cardiac RSK3 Inhibitors, LLC, Los Altos, CA (US)

(72) Inventors: Michael S. KAPILOFF, Los Altos, CA (US); Federico Cividini, Belmont, CA (US)

(73) Assignee: Cardiac RSK3 Inhibitors, LLC, Los Altos, CA (US)

(21) Appl. No.: 18/439,546

(22) Filed: Feb. 12, 2024

Related U.S. Application Data
(60) Provisional application No. 63/484,543, filed on Feb. 13, 2023.

(51) Int. Cl. C07K 14/47; A61K 48/00; C12N 15/86

(52) U.S. Cl. CPC C07K 14/4716; A61K 48/0025; C12N 15/86; C12N 2750/14143; C12N 2800/60

(57) ABSTRACT

The present invention describes a novel gene regulatory sequence containing promoter and enhancer sequences of the human cardiac troponin T gene (TNNT2) that directs expression selectively in the cardiac myocyte. The new TNNT2 promoter/enhancer composition can be used to direct adeno-associated virus gene expression or to construct cell-type specific expression vectors or for cardiac specific transgenesis. The use of this new promoter/enhancer composition is demonstrated by expression of an mAKAP shRNA and mAKAP-derived anchoring disruptor peptides useful for the treatment of heart failure.

### CROSS-REFERENCE TO RELATED APPLICATIONS

This application claims priority to U.S. Provisional Application Ser. No. 63/484,543, filed Feb. 13, 2023, which is incorporated by reference in its entirety for all purposes. The present application also incorporates by reference in their entireties for all purposes U.S. Pat. Nos. 9,132,174, 9,937,228, 10,617,737, 11,229,679 and U.S. application Ser. No. 17/580,692, filed Jan. 21, 2022, as well as U.S. Pat. No. 10,907,153 and U.S. application Ser. No. 16/818,771, filed Mar. 13, 2020.

### STATEMENT OF GOVERNMENTAL SUPPORT

This invention was made with Government support under contracts R44 HL158318 and HL147631 awarded by the National Institutes of Health. The Government has certain rights in this invention.

### REFERENCE TO SEQUENCE LISTING

The instant application contains a Sequence Listing which has been submitted electronically in XML format and is hereby incorporated by reference in its entirety. Said XML copy, created on Feb. 7, 2024, is named 65274_4US01_generic_SL.xml and is 292,801 bytes in size.

### BACKGROUND OF THE INVENTION

In response to chronic stress, the heart's main compensatory mechanism is myocyte hypertrophy, a non-mitotic increase in volume of the contractile cells (Hill and Olson 2008). The adult mammalian myocyte is roughly cylindrical and can grow either in width or length. Because myocytes contribute the vast majority of the myocardial mass of the heart (Jugdutt 2003), concentric and eccentric hypertrophy of the cardiac myocyte result in thickening of heart chamber walls and dilation of the chambers, respectively. In theory, “concentric” myocyte growth in width involving parallel assembly of sarcomeres reduces ventricular wall stress (Law of LaPlace), while “eccentric” lengthwise myocyte growth involving serial assembly of sarcomeres may accommodate greater ventricular volumes without stretching individual sarcomeres beyond the optimum length for contraction (length-tension relationship) (Grossman, Jones, and McLaurin 1975). While the left ventricle will undergo relatively symmetric hypertrophy in response to physiologic stress such as pregnancy or exercise training, concentric ventricular hypertrophy is the predominant initial response to the increased systolic wall stress present in pressure overload diseases such as hypertension or aortic stenosis. Eccentric ventricular hypertrophy predominates during states of volume overload such as occurs following myocardial infarction, as well as during the transition from concentric hypertrophy to the dilated heart in Heart Failure with Reduced Ejection Fraction (HFrEF) in some forms of cardiovascular disease, including diseases mainly characterized by pressure overload. Concentric and Eccentric hypertrophy are also present in inherited hypertrophic and dilated cardiomyopathies, respectively.

At the cellular level, cardiac myocyte hypertrophy occurs as the result of an increase in protein synthesis and in the size and organization of sarcomeres within individual myocytes. For a more thorough review of cardiac remodeling and hypertrophy, see (Nakamura and Sadoshima 2018) and (Burchfield, Xie, and Hill 2013), each herein incorporated by reference in their entirety. The prevailing view is that cardiac hypertrophy plays a major role in the development of heart failure. Traditional routes of treating heart failure include afterload reduction, blockage of beta-adrenergic receptors (β-ARs) and use of mechanical support devices in afflicted patients. However, the art is in need of additional mechanisms of preventing or treating pathological cardiac hypertrophy.

Research suggests that mechanisms that induce “compensatory” concentric hypertrophy early in pressure-overload related heart disease predispose the heart to later systolic dysfunction and eventual failure (Schiattarella and Hill 2015). In this regard, results show that targeting of RSK3-mAKAPβ complexes will attenuate cardiac remodeling due to pressure overload and prevent heart failure (Kritzer et al. 2014; Li, Kritzer, et al. 2013; Li et al. 2020). Accordingly, inhibition of signaling pathways that induce remodeling, including concentric hypertrophy, may be desirable early in pressure overload disease. Conversely, it is also possible that efforts to maintain signals that may promote concentric hypertrophy and oppose eccentric hypertrophy would preserve cardiac volumes and contractility when initiated when the heart is at a stage in the disease process characterized by the eccentric growth and ventricular dilatation leading to HFrEF, whether late in pressure overload-related disease or throughout the progression of volume overload-related disease. In this regard, results show that targeting of PP2A-mAKAPβ complexes will attenuate cardiac remodeling after myocardial infarction (Martinez et al. 2022; Li et al. 2020). Accordingly, it is possible that the enhancement of concentric myocyte hypertrophy and/or the inhibition of eccentric myocyte hypertrophy in familial dilated cardiomyopathy may be beneficial.

### AKAPs and Cardiac Remodeling

Ventricular myocyte hypertrophy is the primary compensatory mechanism whereby the myocardium reduces ventricular wall tension when submitted to stress because of myocardial infarction, hypertension, and congenital heart disease or neurohumoral activation. It is associated with a nonmitotic growth of cardiomyocytes, increased myofibrillar organization, and upregulation of specific subsets of “fetal” genes that are normally expressed during embryonic life (Nakamura and Sadoshima 2018). The concomitant aberrant cardiac contractility, Ca²⁺ handling, and myocardial energetics are associated with maladaptive changes that include interstitial fibrosis and cardiomyocyte death and increase the risk of developing heart failure and malignant arrhythmia (Xie, Burchfield, and Hill 2013; Burchfield, Xie, and Hill 2013). Together, these adaptations contribute to both systolic and diastolic dysfunction that are present in different proportions depending upon the underlying disease (Sharma and Kass 2014). Pathological remodeling of the myocyte is regulated by a complex intracellular signaling network that includes mitogen-activated protein kinase (MAPK), cyclic nucleotide, Ca²⁺, hypoxia, and phosphoinositide-dependent signaling pathways (Heineke and Molkentin 2006; Nakamura and Sadoshima 2018).

Increased in prevalence by risk factors such as smoking and obesity, in the United States, heart failure affects 6.7 million adults, and each year ˜1,000,000 new adult cases are diagnosed (Tsao et al. 2023). The prevalence and incidence of heart failure are increasing, mainly because of increasing life span, but also because of the increased prevalence of risk factors (hypertension, diabetes, dyslipidemia, and obesity) and improved survival rates from other types of cardiovascular disease (myocardial infarction [MI] and arrhythmias) (Heidenreich et al. 2013). First-line therapies for heart failure include β-adrenergic, angiotensin II, and mineralocorticoid receptor antagonists, angiotensin-converting enzyme and neprilysin metalloprotease inhibitors, and sodium-glucose co-transporter 2 inhibitors (Heidenreich et al. 2022). Subsequent or alternative therapies include loop and thiazide diuretics, vasodilators, and I_{f} current blockers, as well as device-based therapies. Nevertheless, the 5-year mortality for symptomatic heart failure remains ˜50%, including >40% mortality for those post-MI (Heidenreich et al. 2013; Gerber et al. 2016).

Cardiac hypertrophy can be induced by a variety of neuro-humoral, paracrine, and autocrine stimuli, which activate several receptor families including G protein-coupled receptors, cytokine receptors, and growth factor tyrosine kinase receptors (Nakamura and Sadoshima 2018). In this context, it is becoming increasingly clear that A-kinase anchoring proteins (AKAPs) can assemble multiprotein complexes that integrate hypertrophic pathways emanating from these receptors. In particular, recent studies have now identified anchoring proteins including mAKAP, AKAP-Lbc, and D-AKAP1 that serve as scaffold proteins and play a central role in organizing and modulating hypertrophic pathways activated by stress signals (Kritzer et al. 2012).

As the organizers of “nodes” in the intracellular signaling network, scaffold proteins are of interest as potential therapeutic targets (Negro, Dodge-Kafka, and Kapiloff 2008). In cells, scaffold proteins can organize multimolecular complexes called “signalosomes,” constituting an important mechanism responsible for specificity and efficacy in intracellular signal transduction (Scott and Pawson 2009). Firstly, many signaling enzymes have broad substrate specificity. Scaffold proteins can co-localize these pleiotropic enzymes with individual substrates, selectively enhancing the catalysis of substrates and providing a degree of specificity not intrinsic to the enzyme's active site (Scott and Pawson 2009). Secondly, some signaling enzymes are low in abundance. Scaffold proteins can co-localize a rare enzyme with its substrate, making signaling kinetically favorable. Thirdly, since many scaffolds are multivalent, scaffold binding can orchestrate the co-regulation by multiple enzymes of individual substrate effectors. Muscle A-kinase anchoring protein (mAKAP, a.k.a. AKAP6) is a large scaffold expressed in cardiac and skeletal myocytes and neurons that binds both signaling enzymes such as protein kinase A (PKA) and the Ca²⁺/calmodulin-dependent phosphatase Calcineurin (CaN) that have broad substrate specificity and signaling enzymes such as p90 ribosomal S6 kinase 3 (RSK3) that is remarkably low in abundance (FIG. 16) (Wang et al. 2015; Pare, Easlick, et al. 2005; Michel et al. 2005; Kapiloff et al. 1999). mAKAPβ is the alternatively-spliced isoform expressed in myocytes, in which cells it is localized to the outer nuclear membrane by binding the integral membrane protein nesprin-1α (Pare, Easlick, et al. 2005).

Consistent with its role as a scaffold protein for stress-related signaling molecules in the cardiac myocyte, depletion of mAKAPβ in rat neonatal ventricular myocytes in vitro inhibited hypertrophy induced by α-adrenergic, β-adrenergic, endothelin-1, angiotensin II, and leucine inhibitor factor/gp130 receptor signaling (Zhang et al. 2011; Pare, Bauman, et al. 2005; Dodge-Kafka et al. 2005; Guo et al. 2015). In vivo, along with attenuating hypertrophy induced by short-term pressure overload and chronic β-adrenergic stimulation, mAKAP gene targeting in the mouse inhibited the development of heart failure following long-term pressure overload, conferring a survival benefit (Kritzer et al. 2014). Specifically, mAKAP gene deletion in the mAKAP^{fl/fl}; Tg(Myh6-cre/Esr1*), tamoxifen-inducible, conditional knock-out mouse reduced left ventricular hypertrophy, while greatly inhibiting myocyte apoptosis, and interstitial fibrosis, left atrial hypertrophy, and pulmonary edema (wet lung weight) due to transverse aortic constriction for 16 weeks (Kritzer et al. 2014).

mAKAP gene targeting is also beneficial following myocardial infarction (Martinez et al. 2022). Permanent ligation of the left anterior descending coronary artery (LAD) in the mouse results in myocardial infarction, including extensive myocyte death, scar formation, and subsequent left ventricular (LV) remodeling. Four weeks following LAD ligation, mAKAP conditional knock-out mouse had preserved LV dimensions and function when to compared to infarcted control cohorts. mAKAP conditional knock-out mice had preserved LV ejection fraction and indexed atrial weight compared to controls, while displaying a remarkable decrease in infarct size.

Introduction to mAKAP and Cardiac Remodeling

mAKAP was originally identified in a cDNA library screen for new cAMP-dependent protein kinase (PKA) regulatory-subunit (R-subunit) binding proteins, i.e. A-kinase anchoring proteins or AKAPs (Mccartney et al. 1995). mAKAP was initially named “AKAP100” for the size of the protein encoded by the original cDNA fragment (Mccartney et al. 1995). Subsequently, the full-length mRNA sequence for mAKAPα, the alternatively-spliced isoform of mAKAP expressed in neurons, was defined, revealing that wildtype mAKAPα is a 255 kDA scaffold (Kapiloff et al. 1999). The sequence for mAKAPβ, the 230 kDa alternatively-spliced isoform of mAKAP expressed in striated myocytes, was later obtained, showing that when expressed in heart or skeletal muscle, mAKAP is translated from an internal start site corresponding to mAKAPα residue Met-245 (Michel et al. 2005).

mAKAP is localized to the nuclear envelope both in neurons, striated cardiac and skeletal myocytes and osteoclasts, the cell types in which mAKAP is clearly expressed (Kapiloff et al. 1999; Pare, Easlick, et al. 2005; Michel et al. 2005; Becker et al. 2021; Vergarajauregui et al. 2020). mAKAP is not a transmembrane domain protein and contains three spectrin-like repeat regions (residues 772-1187) that confer its localization (Kapiloff et al. 1999). Binding of mAKAP's third spectrin repeat (residues 1074-1187) by the outer nuclear membrane protein nesprin-1α is both necessary and sufficient for mAKAP outer nuclear membrane localization, at least in myocytes and when expressed in heterologous cells (Pare, Easlick, et al. 2005; Becker et al. 2021; Holt et al. 2019). Interestingly, mutations in lamin A/C, emerin, and nesprin-1α have been associated with Emery-Dreyfuss muscular dystrophy, as well as other forms of cardiomyopathy (Bonne et al. 1999; Fatkin et al. 1999; Muchir et al. 2000; Bione et al. 1994; Zhang et al. 2007). However, no disease-causing mutations have yet been identified in the human mAKAP gene, and mAKAPβ knock-out in the mouse heart early in development does not induce cardiomyopathy (Kritzer et al. 2014). Besides binding nesprin-1α, mAKAPβ also binds phospholipase Cε (PLCε) through mAKAP's first spectrin repeat, potentially strengthening its association with the nuclear envelope (Zhang et al. 2011). There were early reports of mAKAPβ being present on the sarcoplasmic reticulum (Mccartney et al. 1995; Marx et al. 2000; Yang et al. 1998), but these findings were likely attributable to technical issues with antibody specificity (Kapiloff, Jackson, and Airhart 2001; Kapiloff et al. 1999).

Besides PKA, PLCε and nesprin-1α, mAKAPβ binds a wide variety of proteins important for myocyte stress responses: adenylyl cyclase type 5 (AC5), exchange protein activated by cAMP-1 (Epac1), cAMP-specific phosphodiesterase type 4D3 (PDE4D3), MEK5 and ERK5 MAP-kinases, 3-phosphoinositide-dependent protein kinase-1 (PDK1), p90 ribosomal S6 kinases 3 (RSK3), protein kinase Cε (PKCε), protein kinase D (PKD1, PKCμ), the protein phosphatases calcineurin (CaN) Aβ and PP2A, the type 2 ryanodine receptor (RyR2), the sodium/calcium exchanger NCX1, ubiquitin E3-ligases involved in HIF1α regulation, and myopodin (Pare, Bauman, et al. 2005; Pare, Easlick, et al. 2005; Dodge-Kafka et al. 2005; Marx et al. 2000; Kapiloff, Jackson, and Airhart 2001; Michel et al. 2005; Li et al.; Wong et al. 2008; Zhang et al. 2011; Dodge-Kafka and Kapiloff 2006; Vargas et al. 2012; Faul et al. 2007; Schulze et al. 2003; Kapiloff et al. 2009; Zhang et al. 2013). Bound to mAKAPβ, these signaling molecules co-regulate the transcription factors hypoxia-inducible factor 1α (HIF1α), myocyte enhancer factor-2 (MEF2), serum response factor (SRF), and nuclear factor of activated T-cell (NFATc) transcription factors, as well as type II histone deacetylases (FIG. 7) (Kritzer et al. 2014; Li, Vargas, et al. 2013; Li et al. 2010; Wong et al. 2008; Li et al. 2019; Dodge-Kafka et al. 2018; Li et al. 2020). Some of these molecules are bound directly and some indirectly, some constitutively and some in a regulated manner. Thus, it is likely that the composition of mAKAPβ signalosomes depends upon the underlying state of the myocyte. As research continues on mAKAPβ, the list of its binding partners grows, confirming its hypothesized role as an important orchestrator of signaling pathways required for remodeling. Most of what is known about mAKAPβ is based upon work using cultured neonatal rat ventricular myocytes, in which mAKAPβ was early on recognized to be required for the induction of hypertrophy by a variety of upstream receptors, including α- and β-adrenergic and cytokine receptors (Pare, Bauman, et al. 2005; Dodge-Kafka et al. 2005). However, recently, the phenotype of a conditional, cardiac-myocyte specific mAKAPβ knock-out mouse has been published confirming the centrality of mAKAPβ to remodeling (Kritzer et al. 2014; Martinez et al. 2022). There are various upstream inputs, downstream effectors (outputs), and integrative circuitry within mAKAPβ signalosomes that impact pathological remodeling of the heart.

mAKAPβ—a Prototypical A-Kinase Anchoring Protein

Like most AKAPs, mAKAP contains an amphipathic helix (residues 2055-2072) responsible for binding PKA (Kapiloff et al. 1999; Kritzer et al. 2012). PKA is a heterotetramer of two R-subunits and two catalytic C-subunits, in the configuration C-R-R-C. Within the holoenzyme, the N-terminal docking and dimerization domains of the PKA R-subunits form a X-type, antiparallel four-helix bundle (Newlon et al. 1999). This bundle contains a hydrophobic groove that accommodates the hydrophobic face of the AKAP amphipathic helix. mAKAPβ binds selectively type II PKA (that contains RII subunits) with high affinity (K_{D}=119 nM) (Zakhary et al. 2000). Interestingly, PKA-mAKAPβ binding is increased 16-fold following RIIα autophosphorylation (Zakhary et al. 2000), potentially affecting PKA-mAKAPβ binding in states of altered β-adrenergic signaling. Besides mAKAPβ, there are over a dozen other AKAPs expressed in the myocyte, each with its own distinct localization and sets of binding partners (Kritzer et al. 2014). Remarkably, mAKAP is one of the rarest AKAPs in the myocyte, such that loss of mAKAP does not even affect the localization of perinuclear PKA (Kapiloff, unpublished observations). Despite the low level of expression of the scaffold, replacement in myocytes of endogenous mAKAPβ with a full-length mAKAPβ mutant that cannot bind PKA is sufficient to inhibit the induction of myocyte hypertrophy (Pare, Bauman, et al. 2005). Thus, mAKAPβ signalosomes serve as an example of both how finely PKA signaling may be compartmentalized even on an individual organelle and how the level of expression of a protein or a protein complex is not necessarily indicative of the functional significance of that protein.

mAKAPβ is remarkable because it binds not only effectors for cAMP signaling, but also enzymes responsible for cAMP synthesis and degradation (Kapiloff et al. 2009; Dodge et al. 2001). The synthesis of cAMP from ATP is catalyzed by adenylyl cyclases (AC), while cAMP metabolism to 5′AMP is catalyzed by phosphodiesterases (PDE). The differential association of ACs and PDEs with AKAPs contributes to cAMP compartmentation in cells, providing both for local activation of cAMP effectors and regulation of local cAMP levels by unique regulatory feedback and feedforward loops (Scott, Dessauer, and Tasken 2013). mAKAP is capable of binding both AC2 and AC5, but AC5 appears to be the relevant mAKAPβ-binding partner in the heart (Kapiloff et al. 2009). The N-terminal, C1 and C2 domains of AC5 bind directly to a unique N-terminal site on mAKAPβ (residues 275-340). AC5 activity is inhibited by PKA feedback phosphorylation that in cells is facilitated by mAKAPβ complex formation (Kapiloff et al. 2009). This negative feedback appears to be physiologically relevant to the maintenance of basal cAMP signaling. When the tethering of AC5 to mAKAPβ is inhibited by a competitive peptide comprising the mAKAP AC5-binding domain, both the cAMP content and size of myocytes were increased in the absence of hypertrophic stimulus (Kapiloff et al. 2009).

mAKAP was the first AKAP shown to bind a PDE (Dodge et al. 2001). A site within mAKAP 1286-1831 binds the unique N-terminal domain of PDE4D3. Phosphorylation of PDE4D3 serine residues 13 and 54 results in increased binding to the scaffold and increased PDE catalytic activity, respectively (Dodge et al. 2001; Sette and Conti 1996; Carlisle Michel et al. 2004). Because increased PDE4D3 activity accelerates cAMP degradation, PKA and PDE4D3 constitute a negative feedback loop that can modulate local cAMP levels and PKA activity (Dodge et al. 2001). PDE4D3 bound to mAKAP serves not only as a PDE, but also as an adapter protein recruiting the MAPKs MEK5 and ERK5 and the cAMP-dependent, Rap1-guanine nucleotide exchange factor Epac1 to the scaffold (Dodge-Kafka et al. 2005). Activation of MEK5 and ERK5 by upstream signals results in PDE4D3 phosphorylation on Ser-579, inhibiting the PDE and promoting cAMP accumulation and PKA activation (Dodge-Kafka et al. 2005; Hoffmann et al. 1999; Mackenzie et al. 2008). Epac1 is less sensitive to cAMP than PKA, such that very high cAMP levels result in the additional activation of mAKAP-associated Epac1. Through Rap1, Epac1 can inhibit ERK5 activity, thus preventing PDE4D3 inhibition by MAPK signaling, resulting presumably in maximal PDE4D3 activity due to concomitant PKA phosphorylation (Dodge-Kafka et al. 2005). As a result, Epac1, ERK5, and PDE4D3 constitute a third negative feedback loop that will attenuate cAMP levels in the vicinity of mAKAP complexes opposing cAMP elevation to extremely high levels.

Additional complexity is afforded by the binding of the serine-threonine phosphatase PP2A to the C-terminus of mAKAP (residues 2083-2319) (Dodge-Kafka et al. 2010). PP2A can catalyze the dephosphorylation of PDE4D3 Ser-54, thereby inhibiting the PDE in the absence of upstream stimulus. PP2A associated with mAKAP complexes contain B56δ B subunits, which are PKA substrates. PKA phosphorylation enhances PP2A catalytic activity (Ahn et al. 2007), such that phosphorylation of B56δ by mAKAP-bound PKA increases PDE4D3 dephosphorylation, inhibiting the PDE. This presumably increases cAMP levels, constituting a positive feedforward loop for the initiation of cAMP signaling. Together with the negative feedback loops based upon AC5 phosphorylation and PDE4D3 regulation by PKA and ERK5, one would predict that cAMP levels at mAKAPβ signalosomes would be tightly controlled by upstream β-adrenergic and MAPK signaling. Signaling upstream of AC5 and ERK5 will promote cAMP signaling that will be initially promoted by PP2A feedfoward signaling, while PDE4D3 activation and AC5 inhibition by PKA and Epac1 negative feedback will constrain signaling. Interestingly, Rababa'h et al. demonstrated how mAKAP proteins containing non-synonymous polymorphisms differentially bound PKA and PDE4D3 (Rababa'h et al. 2013). The potential for cAMP signaling to be differentially modulated by crosstalk between upstream signaling pathways or by human polymorphisms makes compelling further work in myocytes to show the relevance of this complicated signaling network. Besides PDE4D3, recently it was shown that mAKAPβ-bound PP2A catalyzes the dephosphorylation of SRF, promoting myocyte growth in length (Li et at. 2020). Targeting of mAKAPβ-bound PP2A improved cardiac function after myocardial infarction in mice.

mAKAPβ and MAP-kinase-RSK3 Signaling

The recruitment of ERK5 by PDE4D3 to mAKAPβ complexes was initially shown to be relevant to the local regulation of cAMP through the aforementioned feedback loops (Dodge-Kafka et al. 2005). However, ERK5 was also recognized to be an important inducer of myocyte hypertrophy, preferentially inducing the growth in length (eccentric hypertrophy) of cultured myocytes, while also being important for concentric hypertrophy in vivo due to pressure overload (transverse aortic constriction in the mouse) (Nicol et al. 2001; Kimura et al. 2010). Notably, inhibition by RNA interference (RNAi) of mAKAPβ expression in cultured myocytes inhibited the eccentric growth induced by the interleukin-6-type cytokine leukemia inhibitory factor (LIF) (Dodge-Kafka et al. 2005). A potential effector for mAKAPβ-bound ERK5 was MEF2 transcription factor, as discussed below. However, in both heart and brain, mAKAP bound PDK1, a kinase that together with ERKs (ERK1, 2 or 5) can activate the MAPK effector p90RSK, a kinase also associated with mAKAP (Ranganathan et al. 2006; Michel et al. 2005). Importantly, binding of PDK1 to mAKAP obviated the requirement for membrane association in RSK activation (Michel et al. 2005). Taken together, these data suggested that mAKAPβ could orchestrate RSK activation in myocytes in response to upstream MAPK signaling.

p90RSK is a pleiotropic ERK effector that regulates many cellular processes, including cell proliferation, survival, migration, and invasion. RSK activity is increased in myocytes by most hypertrophic stimuli (Anjum and Blenis 2008; Sadoshima et al. 1995). In addition, RSK activity was found to be increased in human end-stage dilated cardiomyopathy heart tissue (Takeishi et al. 2002). RSK family members contain 2 catalytic domains, an N-terminal kinase domain and a C-terminal kinase domain (Anjum and Blenis 2008). The N-terminal kinase domain phosphorylates RSK substrates and is activated by sequential phosphorylation of the C-terminal and N-terminal kinase domain activation loops by ERK and PDK1, respectively, such that PDK1 phosphorylation of the N-terminal domain on Ser-218 is indicative of full activation of the enzyme. There are 4 mammalian RSK family members that are ubiquitously expressed, but only RSK3 binds mAKAPβ (Li, Kritzer, et al. 2013). The unique N-terminal domain of RSK3 (1-30) binds directly mAKAPβ residues 1694-1833, called RSK3-binding domain (“RBD”), explaining the selective association of that isoform with the scaffold (Li, Kritzer, et al. 2013). Despite the fact that RSK3 is expressed less in myocytes than other RSK family members, neonatal myocyte hypertrophy was found to be attenuated by RSK3 RNAi, inactivation of the RSK3 N-terminal kinase domain, and disruption of RSK3 binding to mAKAP using an anchoring disruptor peptide (Li, Kritzer, et al. 2013). Importantly, RSK3 expression in vivo was required for the induction of cardiac hypertrophy by both pressure overload and catecholamine infusion, as well as for the heart failure associated with a mouse model for familial hypertrophic cardiomyopathy (α-tropomyosin Glu180Gly) (Li, Kritzer, et al. 2013; Passariello et al. 2013). In addition, consistent with the reported role of ERK1/2 MAP-Kinase in selectively inducing concentric hypertrophy (Kehat et al. 2011), RSK3 gene deletion inhibited the concentric hypertrophy induced by Raf1^{L613V} mutation in a mouse model for Noonan Syndrome (Passariello et al. 2016). The recognition that this specific RSK isoform is required for cardiac remodeling makes it a compelling candidate for therapeutic targeting.

Recently RSK3 at mAKAPβ was shown to be phosphorylate SRF transcription factor, promoting cardiac myocyte growth in width (Li et al. 2020). Displacement of RSK3 from mAKAPβ by the expression of the RBD peptide in mice inhibited the development of hypertrophy in response to pressure overload and the subsequent development of heart failure.

mAKAPβ and Phosphatidylinositide Signaling

The cAMP effector Epac1 activates Rap1 at mAKAPβ complexes affecting ERK5 signaling (Dodge-Kafka et al. 2005). In addition, Epac1-Rap1 activates PLCε, a phospholipase whose Ras association domains directly bind the first spectrin repeat-like domain of mAKAPβ (Zhang et al. 2011). Like mAKAPβ, PLCε was required for neonatal myocyte hypertrophy, whether inhibited by RNAi or by displacement from mAKAPβ by expression of competitive binding peptides. In an elegant paper by the Smrcka laboratory, mAKAPβ-bound PLCε has been shown to regulate PKCε and PKD activation through a novel phosphatidylinositol-4-phosphate (PI4P) pathway in which PLCε selectively converts perinuclear PI4P to diacylglycerol and inositol-1,4-bisphosphate (Zhang et al. 2013). PKD1 phosphorylates type II histone deacetylases (HDACs 4/5/7/9) inducing their nuclear export and de-repressing hypertrophic gene expression (Monovich et al. 2010; Xie and Hill 2013). Smrcka and colleagues found that PLCε was required for pressure overload-induced PKD activation, type II HDAC phosphorylation and hypertrophy in vivo (Zhang et al. 2013). Subsequently, mAKAPβ was also found to be is required in vivo for PKD activation and HDAC4 phosphorylation in response to pressure overload (Kritzer et al. 2014). Remarkably, mAKAPβ can form a ternary complex with PKD and HDAC4. Together, these results show how local cAMP signaling can affect the regulation of cardiac gene expression.

Recently it was published that mAKAPβ is a scaffold for HDAC5 in cardiac myocytes, forming signalosomes containing HDAC5, PKD, and PKA (Dodge-Kafka et al. 2018). Inhibition of mAKAPβ expression attenuated the phosphorylation of HDAC5 by PKD and PKA in response to α- and β-adrenergic receptor stimulation, respectively. Importantly, disruption of mAKAPβ-HDAC5 anchoring prevented the induction of HDAC5 nuclear export by α-adrenergic receptor signaling and PKD phosphorylation. In addition, disruption of mAKAPβ-PKA anchoring prevented the inhibition by β-adrenergic receptor stimulation of α-adrenergic-induced HDAC5 nuclear export. Together, these data establish that mAKAPβ signalosomes serve to bidirectionally regulate the nuclear-cytoplasmic localization of class IIa HDACs. Thus, the mAKAPβ scaffold serves as a node in the myocyte regulatory network controlling both the repression and activation of pathological gene expression in health and disease, respectively.

mAKAPβ and Calcium Signaling

Besides cAMP, phosphoinositide and MAP-kinase signaling, mAKAPβ contributes to the orchestration of Ca²⁺-dependent signaling transduction. The second binding partner for mAKAPβ identified was the ryanodine receptor Ca²⁺ release channel (RyR2) responsible for Ca²⁺-induced Ca²⁺ release from intracellular stores (Kapiloff, Jackson, and Airhart 2001; Marx et al. 2000). RyR2 is best known for its role in excitation-contraction coupling, in which bulk Ca²⁺ is released to induce sarcomeric contraction. PKA phosphorylation can potentiate RyR2 currents (Valdivia et al. 1995; Dulhunty et al. 2007; Bers 2006), although the importance of PKA-catalyzed RyR2 phosphorylation to excitation-contraction coupling is highly controversial (Houser 2014; Dobrev and Wehrens 2014). A small fraction of RyR2, presumably located at perinuclear dyads (Escobar et al. 2011), can be immunoprecipitated with mAKAPβ and nesprin-1α antibodies (Pare, Easlick, et al. 2005; Kapiloff, Jackson, and Airhart 2001). By associating with RyR2, mAKAPβ and nesprin-1α appear to bring together elements of the excitation-contraction coupling machinery and signaling molecules important for regulating nuclear events germane to pathological remodeling. β-adrenergic stimulation of primary myocyte cultures results in increased PKA phosphorylation of mAKAPβ-associated RyR2 (Pare, Bauman, et al. 2005). Notably, only the phosphorylation by PKA of RyR2 associated with nesprin-1α at the outer nuclear membrane and not RyR2 elsewhere on the sarcoplasmic reticulum is dependent upon mAKAPβ (Turcotte et al. 2022). PKA-catalyzed RyR2 phosphorylation may potentiate local Ca²⁺ release within the vicinity of mAKAPβ signalosomes during states of elevated sympathetic stimulation. Notably, β-adrenergic stimulated elevation of Ca²⁺ levels near nesprin-1α at the outer nuclear envelope but not in the general cytosol is dependent upon mAKAPβ expression in cardiac myocytes (Turcotte et al. 2022).

While the few mAKAPβ-associated RyR2s do not apparently affect overall myocyte contractility, a target for increased perinuclear Ca²⁺ is the Ca²⁺/calmodulin-dependent phosphatase calcineurin (CaN) that can bind the scaffold (Turcotte et al. 2022). There are three isoforms of the catalytic subunit for CaN (α,β,γ), but only CaNAβ-mAKAPβ complexes have been detected in myocytes (Li et al. 2010). Remarkably, CaNAβ is the CaNA isoform important for the induction of cardiac hypertrophy in vivo, as well as for myocyte survival after ischemia (Bueno et al. 2002; Bueno et al. 2004). CaNAβ binds directly to a unique site within mAKAPβ (residues 1286-1345) (Pare, Bauman, et al. 2005; Li et al. 2010). CaNAβ binding to mAKAPβ is enhanced in cells by adrenergic stimulation and directly by Ca²⁺/calmodulin (Li et al. 2010). Notably, CaNAβ-mAKAPβ binding was required for α-adrenergic-induced neonatal myocyte hypertrophy in vitro (Li et al. 2010).

mAKAPβ and Gene Expression

Among its many substrates, CaN is responsible for the activation of NFATc and MEF2 transcription factors. The NFATc transcription factor family includes four CaN-dependent isoforms that are all expressed in myocytes and that can contribute to the induction of myocyte hypertrophy (Wilkins et al. 2004). In general, NFATc family members are retained in the cytoplasm when heavily phosphorylated on the multiple serine-rich motifs within the N-terminal regulatory domain. NFATc translocates into the nucleus when these motifs are dephosphorylated by CaN. Multiple NFATc family members can bind mAKAPβ, and binding to mAKAPβ was required for CaN-dependent dephosphorylation of NFATc3 in myocytes (Li et al. 2010). Accordingly, mAKAPβ expression was also required for NFAT nuclear translocation and transcriptional activity in vitro (Li et al. 2010; Pare, Bauman, et al. 2005). These results correlate with observations that NFAT-dependent gene expression in vivo was attenuated by mAKAPβ cardiac-myocyte specific knock-out following transverse aortic constriction (Kritzer et al. 2014).

Like NFATc2 and NFATc3, MEF2D is a transcription factor required for cardiac hypertrophy in vivo (Kim et al. 2008; Wilkins et al. 2002; Bourajjaj et al. 2008). MEF2 family members contain a conserved DNA binding domain that includes both a MADS box and a MEF2 homology domain (Potthoff and Olson 2007). The DNA-binding domain of MEF2D binds directly to an N-terminal domain of mAKAP (Vargas et al. 2012; Kim et al. 2008). CaN and MEF2D are important not only in the heart, but also in skeletal muscle (Naya et al. 1999; Naya and Olson 1999; Black and Olson 1998; Friday et al. 2003; Wu et al. 2001). Interference with MEF2-mAKAPβ binding blunted MEF2 transcriptional activity and the expression of endogenous MEF2 target genes in C2C12 skeletal myoblasts (Vargas et al. 2012). In addition, disruption of MEF2-mAKAP complexes attenuated the differentiation of C2C12 myoblasts into myotubes, as evidenced by decreased cell fusion and expression of differentiation markers (Vargas et al. 2012). Remarkably, CaN-MEF2 binding is mAKAPβ-dependent in cardiac myocytes (Li, Vargas, et al. 2013). Accordingly, disruption of CaN-mAKAPβ binding inhibited both MEF2 transcriptional activity in C2C12 cells and cardiac myocyte hypertrophy (Li, Vargas, et al. 2013). Like NFATc2, MEF2D de-phosphorylation in vivo in response to pressure overload was attenuated following mAKAPβ conditional knock-out, correlating with the decreased expression MEF2-target genes, including the expression of atrial natriuretic factor (Kritzer et al. 2014).

The regulation of NFATc, MEF2 and HDAC4 by mAKAPβ in vivo during pressure overload shows the importance of mAKAPβ to stress-regulated gene expression (Kritzer et al. 2014). Published reports show how, at mAKAPβ, NFATc and MEF2 are regulated by CaN, while HDAC4 and HDAC5 are regulated by PKD and PKA (Li, Vargas, et al. 2013; Zhang et al. 2013; Li et al. 2010; Dodge-Kafka et al. 2018). mAKAPβ appears to facilitate the modulation of these gene regulatory proteins by other signaling enzymes. For example, mAKAPβ-associated ERK5 may phosphorylate MEF2, activating the transcription factor (Kato et al. 2000). In addition, PKA can phosphorylate MEF2, affecting its DNA-binding affinity (Wang et al. 2005). On the other hand, the Olson group has proposed that PKA phosphorylation of HDAC4 can inhibit MEF2 activity through the generation of a novel HDAC4 proteolytic fragment (Backs et al. 2011). How the activities of the many mAKAPβ binding partners are ultimately integrated to control gene expression can be investigated both in vitro and in vivo.

Other mAKAPβ Binding Partners

There are other binding partners for mAKAPβ for whom the significance of docking to the scaffold remains poorly characterized, including phospholamban, myopodin and NCX1 (Faul et al. 2007; Schulze et al. 2003; Hakem Zadeh et al. 2019). AKAP9 and pericentric binding to mAKAPβ have been described in striated myocytes and osteoclasts and are important for the organization of nuclear envelope microtubute-organizing centers and the proximation of the nuclear envelope with the Golgi apparatus (Becker et al. 2021; Vergarajauregui et al. 2020).

HIF-1α, a transcription factor that regulates systemic responses to hypoxia, also binds mAKAPβ (Wong et al. 2008). Under normoxic conditions, the abundance of HIF-1α in the cell is kept low by ubiquitin-mediated proteasomal degradation. HIF-1α is hydroxylated by a family of oxygen-sensitive dioxygenases called prolyl hydroxylases (PHD1, PHD2, and PHD3) (Ohh et al. 2000). Hydroxylated HIF-1α is subsequently recognized by the von Hippel-Lindau protein (pVHL), which recruits the Elongin C ubiquitin ligase complex to ubiquitinate HIF-1α and to promote its proteasome-dependent degradation (Maxwell et al. 1999). Under hypoxic conditions, PHDs are inactivated, HIF-1α degradation is decreased and HIF-1α accumulates in the nucleus, where it can dimerize with HIF-1β to promote the transcription of target genes. mAKAPβ can assemble a signaling complex containing HIF-1α, PHD, pVHL and the E3 ligase Siah2 (seven in absentia homolog 2) in cultured neonatal myocytes (Wong et al. 2008). Under normoxic conditions, mAKAPβ-anchored PHD and pVHL favor HIF-1α ubiquitination and degradation (Wong et al. 2008). Under hypoxic conditions, however, Siah2 activation induces proteasomal degradation of bound PHD, favoring HIF-1α accumulation (Wong et al. 2008). An mAKAPβ knock-out may affect cardiac myocyte survival after ischemia-reperfusion.

mAKAPβ—a Conductor of the Remodeling Symphony

The above discussion shows how multiple signaling pathways known to be important for cardiac hypertrophy and pathological remodeling are modulated by the binding of key signaling intermediates to the mAKAPβ scaffold. Cardiac myocyte-specific, conditional mAKAP knock-out mouse has been characterized, showing the relevance of mAKAPβ signalosomes in vivo (Kritzer et al. 2014; Martinez et al. 2022). mAKAPβ was required in cardiac myocytes for the induction of cardiac hypertrophy by transverse aortic constriction and isoproterenol infusion. Most remarkable, however, was the prevention of pathological remodeling, including myocardial apoptosis and interstitial fibrosis, and the preservation of cardiac function in the face of long-term pressure overload, together resulting in a significant increase in mouse survival (Kritzer et al. 2014). Beneficial results in mice were also shown after myocardial infarction (Martinez et al. 2022). These results established mAKAPβ as the first scaffold whose ablation confers a survival benefit in heart disease. Importantly, mAKAPβ did not appear to be necessary for either the development or maintenance of normal adult cardiac function, as the use of a Nkx2-5-directed cre deleter line did not result in an overt phenotype by six months of age (Kritzer et al. 2014). Although mAKAPβ knock-out did attenuate the physiological hypertrophy induced by forced exercise (swimming), the targeting of mAKAPβ complexes in disease remains relevant.

Various strategies for targeting mAKAPβ complexes in humans may be envisioned, including siRNA knock-down of the scaffold. However, a relatively detailed understanding of the structure and function of mAKAPβ signalosomes provides us with additional approaches to targeting these pathways. For example, the expression of peptides targeting key protein-protein interactions involving mAKAPβ has already been shown to be effective in vitro, including anchoring disruptor peptides targeting mAKAPβ-CaNAβ, mAKAPβ-MEF2D, mAKAPβ-PLCε, mAKAPβ-PP2A and mAKAPβ-RSK3 binding (Li, Vargas, et al. 2013; Li, Kritzer, et al. 2013; Vargas et al. 2012; Zhang et al. 2011; Li et al. 2020). A leading cause of death, heart failure is a disease that incurs 50% mortality within 5 years of diagnosis despite modern therapy, at a cost of over $30 billion/year in the USA alone (Go et al. 2014). Proof-of-concept for the efficacy of targeting mAKAPβ signalosomes by targeting mAKAPβ-PP2A and mAKAPβ-RSK3 binding using adeno-associated virus (AAV) gene therapy vectors has recently been shown in mice (Li et al. 2020). Many candidates for potential targeting in cardiac disease are pleiotropic, complicating the development of drugs with sufficient specificity in vivo. The specific targeting of mAKAPβ signalosomes in the cardiac myocyte provides an opportunity to target relatively rare protein-protein interactions that appear to be dedicated to pathological cardiac remodeling and whose ablation may be promoted without significant side-effects.

### Adeno-Associated Viruses (AAV)

Adeno-associated viruses (AAV) are small single-stranded DNA viruses with a genome of ˜4.7 kb that belong to the family Parvoviridae and the genus Dependoparvovirus (Wang, 2019). Due to their lack of pathogenicity and their ability to deliver essentially any DNA sequence to tissues of interest in mammalian species, AAV have become the gene therapy vectors of choice for both basic research and clinical applications. Since the approval of alipogene tiparvovec (Glybera) for lipoprotein lipase deficiency in Europe in 2012, voretigene neparvovec-rzyl (Luxturna) for RPE365^{-/-} Leber congenital amaurosis and retinitis pigmentosa in the USA in 2017, and onasemnogene abeparvovec (Zolgensma) for spinal muscular atrophy in the USA in 2019, the number of clinical trials for AAV-based therapies has increased dramatically, such that over 200 clinical trials have been registered to date (Kuzmin et al., 2021). Adeno-associated virus species are designated by the serotype of their capsid proteins. Of the commonly used serotypes, AAV serotype 9 (AAV9) has been used frequently for skeletal, cardiac, and neural applications, including the AAV9 biologic drug onasemnogene abeparvovec. For the heart, AAV9 has become the dominant serotype in preclinical research (Kieserman et al., 2019).

An important question during the development of any drug is biodistribution. Despite the recognition that AAV9 delivered via the vascular system will be delivered to other organs, there are few reports regarding the biodistribution of AAV9 in swine or its dependence on local or peripheral sites of introduction. AAV, including serotype 9, have a significant tropism for the liver and in the case of infusion via coronary artery, there is significant off-site hepatic delivery of AAV9 biologics (Ishikawa et al., 2018). Preclinical testing for muscle-directed therapies often includes translation from rodents to swine to reveal potential clinical relevance. Demonstrating the flexibility of AAV gene therapy, AAV9 biologics have been studied in swine for inhibitor-1c and S100A1 protein expression in heart failure (Fish et al., 2013; Pleger et al., 2011) somatic gene editing in Duchenne muscular dystrophy (Moretti et al., 2020) and for RNA interference of the Hippo signal pathway in cardiac regeneration (Liu et al., 2021). Adeno-associated virus has been delivered to the heart by direct intramyocardial injection (Liu et al., 2021) and intracoronary artery infusion (Fish et al., 2013). Intracoronary infusion by percutaneous catheter-based approaches has been performed using a variety of methods. Retrograde techniques have been performed with or without coronary artery/venous occlusion or vector recirculation, while antegrade delivery is perhaps the simplest intracoronary artery approach and readily applicable to patients undergoing standard cardiac catheterization (Ishikawa et al., 2018).

Further discussion of the design and use of AAV vectors can be found, e.g., in U.S. Pat. Nos. 11,129,908 and 5,139,941 and in WO 1998/046728, which are hereby incorporated by reference in their entireties for all purposes. In addition, use of AAV vectors in treating heart disease is also disclosed in U.S. Pat. Nos. 9,132,174, 9,937,228, 10,617,737, 11,229,679 and U.S. application Ser. No. 17/580,692, filed Jan. 21, 2022, as well as U.S. Pat. No. 10,907,153 and U.S. application Ser. No. 16/818,771, filed Mar. 13, 2020, and in Li et al., 2022; Li et al., 2020, and Martinez, et al., 2023, all of which are hereby incorporated herein by reference in their entireties for all purposes.

There is a clear need to develop new effective therapies to treat patients with heart failure, as well as to prevent its development in the context of other cardiovascular diseases such coronary artery disease, hypertension, and valvular disease.

### SUMMARY OF THE INVENTION

The following brief summary is not intended to include all features and aspects of the present invention, nor does it imply that the invention must include all features and aspects discussed in this summary.

The present inventors have discovered improved compositions for and methods of delivering a transgene to a cardiac myocyte using an AAV plasmid containing a novel promoter-enhancer regulatory sequence that contains a novel combination of promoter, exonic, and intronic sequences from the human TNNT2 gene, to direct expression of the transgene.

In some aspects, these improved compositions and methods can be used to treat cardiac pathological processes by inhibiting the signaling properties of individual mAKAP signaling complexes using drugs that target unique protein-protein interactions. Such a therapeutic strategy offers an advantage over classical therapeutic approaches because it allows the selective inhibition of defined cellular responses.

In certain aspects, the compositions and methods of the invention are able to disrupt mAKAP-mediated protein-protein interactions to inhibit the ability of mAKAP to coordinate the activation of enzymes that play a central role in activating key transcription factors that initiate cellular processes leading to pathological cardiac remodeling. Inhibiting the binding activity of mAKAPβ can protect the heart from damage leading to heart failure, for example, following myocardial infarction.

In one aspect, the invention relates to compositions and methods for decreasing expression levels of mAKAPβ, for example, using AAV vectors containing a novel promoter-enhancer regulatory sequence that provide shRNA molecules against mAKAPβ.

In another aspect, the invention relates to compositions and methods for inhibiting the interaction of mAKAPβ and RSK3, for example, using AAV vectors containing a novel promoter-enhancer regulatory sequence and sequences that target the RSK3 binding domain (RBD) of mAKAPβ.

In yet another aspect, the invention relates to compositions and methods for inhibiting the interaction of mAKAPβ and PP2A, for example, using AAV vectors containing a novel promoter-enhancer regulatory sequence and sequences that target the PP2A binding domain (PBD) of mAKAPβ.

In still another aspect, the invention relates to vectors comprising a cardiac troponin t promoter 2 (hTNNT) and a skeletal muscle enhancer with a splicing consensus site.

In a further aspect, the invention relates to vectors that have been codon-optimized and methods of using the same.

The aforementioned compositions and methods can be used for protecting the heart from damage, by administering to a patient at risk of such damage, a pharmaceutically effective amount of a composition which inhibits the expression of, the binding of, and/or activity mAKAPβ.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a design for an AAV plasmid containing a novel promoter-enhancer regulatory sequence (TNNT2 Regulatory Sequence in green), that contains a novel combination of promoter, exonic, and intronic sequences from the human TNNT2 gene, to direct expression of a human mAKAP shRNA.

FIGS. 2-1 to 2-4 show a nucleic acid sequence (SEQ ID NO:1) for the AAV plasmid of FIG. 1.

FIG. 3 shows a design for an AAV plasmid containing a novel promoter-enhancer regulatory sequence (TNNT2 Regulatory Sequence in green), that contains a novel combination of promoter, exonic, and intronic sequences from the human TNNT2 gene, to direct expression of a human mAKAP RBD sequence.

FIGS. 4-1 to 4-3 show a nucleic acid sequence (SEQ ID NO:2) for the AAV plasmid of FIG. 3.

FIG. 5 shows a design for an AAV plasmid containing a novel promoter-enhancer regulatory sequence (TNNT2 Regulatory Sequence in green), that contains a novel combination of promoter, exonic, and intronic sequences from the human TNNT2 gene, to direct expression of a human mAKAP PBD sequence.

FIGS. 6-1 to 6-3 show a nucleic acid sequence (SEQ ID NO:3) for the AAV plasmid of FIG. 5.

FIG. 7 shows a design for an AAV plasmid containing a novel promoter-enhancer regulatory sequence, that contains a novel combination of a human calsequestrin enhancer and promoter and exonic sequences from the human TNNT2 gene, to direct expression of a human mAKAP shRNA.

FIGS. 8-1 to 8-3 show a nucleic acid sequence (SEQ ID NO:4) for the AAV plasmid of FIG. 7.

FIG. 9 shows a design for an AAV plasmid containing a novel promoter-enhancer regulatory sequence, that contains a novel combination of a human calsequestrin enhancer rand promoter and exonic sequences from the human TNNT2 gene, to direct expression of a human mAKAP RBD sequence.

FIGS. 10-1 to 10-3 show a nucleic acid sequence (SEQ ID NO:5) for the AAV plasmid of FIG. 9.

FIG. 11 shows a design for an AAV plasmid containing a novel promoter-enhancer regulatory sequence, that contains a novel combination of a human calsequestrin enhancer and promoter and exonic sequences from the human TNNT2 gene, to direct expression of a human mAKAP PBD sequence.

FIGS. 12-1 to 12-3 show a nucleic acid sequence (SEQ ID NO:6) for the AAV plasmid of FIG. 11.

FIG. 13 shows a nucleotide alignment view of newly introduced silent single nucleotide mutations in human PBD (mAKAP 2132-2319) open reading frame to reduce the amount of CpG containing immune-stimulatory motifs.

FIG. 14 shows nucleotide alignment view of newly introduced silent single nucleotide mutations in human RBD (mAKAP 1696-1835) open reading frame to reduce the amount of CpG containing immune-stimulatory motifs.

FIGS. 15-1 to 15-3 show the origin of the “hTNNT2 Regulatory Sequences” included in the plasmids shown in FIGS. 1-6.

FIG. 16 shows a model for mAKAPβ-regulated, SRF-dependent gene expression. Anchored RSK3 is a Gq-protein coupled receptor-ERK effector that phosphorylates SRF associated with perinuclear mAKAPβ complexes. mAKAPβ-anchored PP2A that can be activated by cAMP-dependent protein kinase A (PKA) opposes SRF phosphorylation. Phosphorylated SRF induces gene expression that promotes concentric hypertrophy.

FIGS. 17-1 to 17-4 show the complete nucleotide (SEQ ID NO:9) and deduced amino acid (SEQ ID NO:10) sequence of human RSK3 (Homo sapiens ribosomal protein S6 kinase A2 (RPS6KA2), transcript variant 1, mRNA, NCBI Reference Sequence: NM_021135.6). The deduced RSK3 protein sequence is indicated in the one-letter amino acid code beginning at the first methionine residue preceding the 733-codon open reading frame and terminating at the asterisk. The unique N-terminal region of RSK3 (which bears no homology to RSK1 or RSK2) is indicated.

FIGS. 18-1 to 18-5 show the nucleotide sequence of human mAKAPα (SEQ ID NO:11) with open reading frame translated (SEQ ID NO:12).

FIGS. 19-1 to 19-2 show the amino acid sequence of human mAKAP (SEQ ID NO:12). mAKAPα starts at residue 1, mAKAPβ at residue 243. PBD in bold.

FIG. 20 shows the amino acid sequence of human PBD (SEQ ID NO:14) as expressed in AAV.

FIG. 21 shows the alignment of the RBD of various species. (SEQ ID NOS: 16-42), with the sequence expressed by the vector in FIG. 3 shown on line 1 (SEQ ID NO:15).

FIG. 22 shows the alignment of the PBD of various species. (SEQ ID NOS:45-64), with the sequence expressed by the vector in FIG. 5 shown on line 1 (SEQ ID NO:68).

FIG. 23 shows a map of a human PBD AAV shuttle plasmid, pscAAV-hmAKAP PBD.

FIGS. 24-1 to 24-2 show the nucleotide sequence of pscAAV-hmAKAP PBD plasmid (SEQ ID NO:67).

FIGS. 25-1 to 25-4 show the sequence of human mAKAP (AKAP6) mRNA (SEQ ID NO:69)-ref seq XM_017021808.1 with shRNA sequences (#1-3) marked. Numbering is for nucleotide sequence. Encoded amino acids are indicated above.

FIG. 26 shows a map of a pscA-TnT-mAKAP shrna (#3) plasmid.

FIGS. 27-1 to 27-2 show nucleotide sequence of pscA-TnT-mAKAP shrna (#3) plasmid (SEQ ID NO:70) with key features and some restriction enzymes sites indicated.

FIG. 28 shows the target for a scAAV-mAKAP shRNA biologic drug. mAKAP mRNA sequences form human (SEQ ID NO:71), swine (SEQ ID NO:72), mouse (SEQ ID NO:73) and rat (SEQ ID NO:74). The boxed sequence is shRNA target #3.

FIG. 29 shows the amino acid sequence of rat mAKAP PBD as expressed in AAV vector (SEQ ID NO:75). Includes N-terminal myc tag.

FIG. 30 shows cardiac-selective delivery of different AAV compositions. Data are mRNA levels in the left ventricle of the heart (LV) relative to that in liver, brain or skeletal muscle (skm). Data were obtained for tissues from 3 swine collected 3 months post iv-infusion of 2E12vg/kg AAV9 vector. RT-qPCR was performed following RNA extraction and reverse transcription (RT) of 2 μg total RNA to cDNA. AAV viral genomes (vg) were quantified by qPCR using genomic DNA extracted from the same tissue. Data shown here were calculated using mRNA levels after normalization to housekeeping gene 18S mRNA levels and for gene delivery to the same tissue. The graph shows fold enrichment of transgene expression averaged over nine different cardiac LV regions (anterior base, lateral base, posterior base, anterior middle, lateral middle, posterior middle, anterior apex, lateral apex, posterior apex) compared to Liver, Cerebral Cortex (Brain), and 5 different skeletal muscles (Tricep Medial head, Tricep long. Head, Tricep lat. Head, Deltoid). “WPRE” refers to AAV generated with the plasmid in FIG. 23 and similar plasmids encoding the human RBD and the mAKAP shRNA. “Calseq” refers to AAV generated with the plasmids in FIGS. 7-12. “enh.int” refers to AAV generated with the plasmids in FIGS. 1-6.

FIGS. 31-1 to 31-4 show the TNNT2 gene sequence (5167 bp excerpted from NCBI chromosome reference NC_000001.11 201378367..201373201: gene is in antisense direction). Exon 1 is at bp 688-745 (NC_000001.11 201377680..201377623) and Exon 2 is at bp 5100-5154 (NC_000001.11 201373268..201373214) of TNNT2 gene.

FIG. 32 shows CaMKII. a) Binding Ca²⁺/calmodulin (CaM) releases CaMKII pseudosubstrate domain autoinhibition. Posttranslational modifications at the N-terminal end of the regulatory domain (exons 11-12 of exons 11-19 (Duran, Nickel et al. 2021)) positively (+) and negatively (−) modulate CaMKII activity. (SEQ ID NO:80). (b) CaMKII dodecameric holoenzymes color coded as in panel a. (PDB IDs 5VLO, 2VN9, and 3SOA) (c) The three CaMKIIδ variants most highly expressed in the adult heart. (SEQ ID NOS:81-82). Figure reproduced from Reyes Gaido, et al (Reyes Gaido, Nkashama et al. 2023).

FIG. 33 shows endogenous mAKAPβ and CaMKII associate in myocytes. Neonatal myocytes expressing myc-GFP or myc-RBD-GFP were used for immunoprecipitation assays with CaMKII and control IgG antibodies. n=3.

FIG. 34 shows mAKAPβ 1694-1833 binds diverse CaMKII isoforms. Myc-tagged RBD and Flag- and mCherry-tagged CaMKII isoforms were co-expressed by transfection of COS-7 cells. Protein complexes were immunoprecipitated using myc-tag antibodies.

FIG. 35 shows RSK3 and CaMKII bind overlapping sites on mAKAPβ. Myc-GFP-tagged mAKAP fragments and CaMKIIγ-mCherry-Flag or mCherry-HA-RSK3 were co-expressed by transfection of COS-7 cells. Protein complexes were immunoprecipitated using myc-tag antibodies. n=2 (RSK3), 3 (CaMKIIγ).

FIG. 36 shows mAKAP 1694-1833 expression inhibits HDAC4 phosphorylation in Ang II-treated neonatal myocytes. HDAC4 was immunoprecipitated from adenoviral-infected myocytes treated for 1 hour with 100 nM Ang II. n=3 biological replicates. The lower band on the p-HDAC4 blot corresponds to HDAC4.

### DETAILED DESCRIPTION OF THE INVENTION

As discussed above, AKAP-based signaling complexes play a central role in regulating physiological and pathological cardiac events. As such, the present inventors have examined inhibiting the signaling properties of individual AKAP signaling complexes using drugs that target unique protein-protein interactions as an approach for limiting cardiac pathological processes. Such a therapeutic strategy offers an advantage over classical therapeutic approaches since it allows the selective inhibition of defined cellular responses.

Anchoring proteins including mAKAPβ are therapeutic targets for the treatment of pathological cardiac hypertrophy and heart failure. In particular, the present inventors have found that disrupting mAKAPβ-mediated protein-protein interactions can be used to inhibit the ability of mAKAPβ to coordinate the activation and function of enzymes that play a central role in activating key transcription factors and chromatin modifying enzymes that initiate and/or promote the remodeling process leading to heart failure.

One aspect of the current invention is that inhibition of cardiac myocyte intracellular signaling that promotes pathological cardiac gene expression, as might be apparent by improvement in in ventricular geometry, will inhibit the progression of heart disease to heart failure. For example, heart failure may be prevented or treated by changes in cardiac myocyte signaling that decrease in eccentric heart disease LV internal diameter by inhibiting the elongation of cardiac myocytes, or, conversely, prevent in concentric heart disease myocyte thickening and increased LV wall thickness. Demonstration of the prevention of cardiac dysfunction has been obtained for gene therapy vectors based upon expression of a muscle A-kinase anchoring protein (mAKAP, a.k.a. AKAP6)-derived: (1) mAKAP shRNAs (see, e.g., U.S. Pat. No. 10,907,153, which is hereby incorporated by reference in its entirety for all purposes, as subsequently published in (Martinez et al. 2022)); (2) RBD peptides (see, e.g., U.S. Pat. Nos. 9,132,174, 9,937,228, 10,617,337, 11,229,679, which are hereby incorporated by reference in their entireties for all purposes, as subsequently published in (Li et al. 2020)); and (3) PBD peptides (see, e.g., U.S. Pat. No. 16,818,771, which is hereby incorporated by reference in its entirety for all purposes, as subsequently published in (Li et al. 2020)).

In particular, the present inventors have discovered novel vectors comprising a human cardiac troponin t 2 (hTNNT2) promoter that demonstrate increases in expression levels of the foregoing molecules in the left ventricle compared to other tissues.

In one embodiment, such vectors also include a skeletal muscle enhancer with a splicing consensus site.

In some embodiments, the vectors also comprise codons optimized for expression and/or decreases immunogenicity.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (4th Ed., 2012); “Current Protocols in Molecular Biology” Volumes I-III [Ausubel, R. M., ed. (1994)]; “Cell Biology: A Laboratory Handbook” Volumes I-III [J. E. Celis, 3rd ed. (2005))]; “Current Protocols in Immunology” Volumes I-III [Coligan, J. E., ed. (2005)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984); C. Machida, “Viral Vectors for Gene Therapy: Methods and Protocols” (2010); J. Reidhaar-Olson and C. Rondinone, “Therapeutic Applications of RNAi: Methods and Protocols” (2009).

The following definitions and acronyms are used herein:
AAV—adeno-associated virus
AC5—adenylyl cyclase type 5
ACE—angiotensin-converting enzyme
ANF atrial natriuretic factor
ARVM—adult rat ventricular myocyte
CaN—calcineurin
CArG box—(CC9AT)₆GG
CPT-cAMP—8-(4-chlorophenylthio)adenosine 3′,5′-cyclic monophosphate
CsA—cyclosporin A
CTKD—C-terminal kinase domain
ERK—extracellular signal-regulated kinase
FBS—fetal bovine serum
Fsk—forskolin
GFP—green fluorescent protein
GPCR—G-protein coupled receptorHDAC—histone deacetylase
Gs—stimulatory G protein
GST—glutathione-S-transferaseHIF1α—hypoxia-inducible factor 1α
HFrEF—heart failure with reduced ejection fraction
IBMX—3-isolbutyl-1-methylxanthine
Iso—isoproterenol
LIF—leukemia inhibitory factor
MADS—(MCM1, agamous, deficiens, SRF) domain—mediates DNA binding to CArG box (CC9AT)₆GG serum response elements (SRE); the MADS-box gene family got its name later as an acronym referring to the four founding members, ignoring ARG80:
   MCM1 from the budding yeast, Saccharomyces cerevisiae,
   AGAMOUS from the thale cress Arabidopsis thaliana,
   DEFICIENS from the snapdragon Antirrhinum majus,[10]
   SRF from the human Homo sapiens.
mAKAP—muscle A-kinase anchoring protein
mAKAPα—alternatively spliced isoform expressed in neurons; 255 kDa
mAKAPβ—alternatively spliced isoform expressed in striated myocytes; 230 kDa
MAPK—mitogen-activated protein kinase
MEF2—myocyte enhancer factor-2
MgAc—magnesium acetate
MI—myocardial infarction
NCX1—sodium/calcium exchanger
NFATc—nuclear factor of activate T-cell
NRVM—neonatal rat ventricular myocyte
NTKD—N-terminal kinase domain
OA—Okadaic acid
PBD—PP2A—anchoring disruptor—attenuates eccentric hypertrophy
PDE4D3—cAMP-specific phosphodiesterase type 4D3
PDK1—3′phosphoinositide-dependent kinase 1
PE—phenylephrine
PHD—prolyl hydroxylase
PI4P—phosphatidylinositol-4-phosphate
PKA—protein kinase A
PKD—protein kinase D
PKI—protein kinase inhibitor
PLCε—phospholipase CF
PKA—cAMP-dependent protein kinase
PP2A—protein (serine-threonine) phosphatase—dephosphorylates SRF Ser¹¹³
PP2B—calcium/calmodulin-dependent protein phosphatase 2B
RBD—isoform-specific N-terminal RSK3 domain binds a discrete “RSK3-binding domain” within mAKAPβ at residues 1694-1833 (RBD)
RSK—p90 ribosomal S6 kinase
RyR2—type 2 ryanodine receptor
scAAV—self-compementary AAV
siRNA—small interfering RNA oligonucleotide
shRNA—short hairpin RNA
SRE—serum response elements
SRF—serum response factor—transcription factor (SRF Ser¹⁰³ phosphorylation induces concentric myocyte and cardiac hypertrophy; inhibition of phosphorylation improves cardiac structure and function)
siRNA—small interfering RNA
TAC—transverse aortic constriction
TCA—trichloroacetic acid
VSV—vesicular stomatitis virus

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well known and commonly used in the art. Certain experimental techniques, not specifically defined, are generally performed according to conventional methods well known in the art and as described in various general and more specific references that are cited and discussed throughout the present specification. For purposes of the clarity, following terms are defined below.

The present invention recognizes that the interaction of RSK3 and/or PP2A and mAKAPβ mediates various intracellular signals and pathways which lead to cardiac myocyte hypertrophy and/or dysfunction. As such, the present inventors have discovered various methods of inhibiting that interaction in order to prevent and/or treat cardiac myocyte hypertrophy and/or dysfunction.

Thus, the present invention includes a method for protecting the heart from damage, by administering to a patient at risk of such damage, a pharmaceutically effective amount of a composition, which inhibits the interaction of RSK3 and/or PP2A and mAKAPβ, or decreases the level of expression of mAKAPβ. It should be appreciated that “a pharmaceutically effective amount” can be empirically determined based upon the method of delivery, and will vary according to the method of delivery.

The invention also relates to a method of treating heart disease, by administering to a patient a pharmaceutically effective amount of a composition, which inhibits the interaction of RSK3 and/or PP2A and mAKAPβ.

The invention also relates to compositions which inhibit the interaction of RSK3 and/or PP2A and mAKAPβ. In particular embodiments, these inhibiting compositions or “inhibitors” include peptide inhibitors, which can be administered by any known method, including by gene therapy delivery. In other embodiments, the inhibitors can be small molecule inhibitors.

Specifically, the present invention is directed to methods and compositions for treating or protecting the heart from damage, by administering to a patient at risk of such damage, a pharmaceutically effective amount of a composition which (1) inhibits the interaction of RSK3 and/or PP2A and mAKAPβ; (2) inhibits the activity of RSK3 and/or PP2A and mAKAPβ; or (3) inhibits the expression of RSK3, PP2A and/or mAKAPβ.

The invention also relates to methods of treating or protecting the heart from damage, by administering to a patient at risk of such damage, a pharmaceutically effective amount of a composition which inhibits a cellular process mediated by the RSK3 and/or PP2A.

In one embodiment, the composition includes an mAKAPβ peptide. In one preferred embodiment, the mAKAPβ peptide is obtained from the carboxy terminus of the mAKAPβ amino acid sequence. In a particularly preferred embodiment, the mAKAPβ peptide is at least a fragment of amino acids 2083-2319 of the mAKAPβ amino acid sequence.

In one preferred embodiment, the mAKAPβ peptide is at least a fragment of amino acids 2133-2319 of the mAKAPβ amino acid sequence.

In one embodiment, the composition includes an mAKAPβ peptide.

In one preferred embodiment, the mAKAPβ peptide comprises nucleotides 1735-1833 of the mAKAP amino acid sequence or a nucleotide sequence having at least 80%, 85%, 90%, 95%, or 99% sequence identity thereto.

In another embodiment, the mAKAPβ peptide is obtained from the carboxy terminus of the mAKAPβ amino acid sequence. In a particularly preferred embodiment, the mAKAPβ peptide is at least a fragment of amino acids 2083-2319 of the mAKAPβ amino acid sequence or a nucleotide sequence having at least 80%, 85%, 90%, 95%, or 99% sequence identity thereto.

In one preferred embodiment, the mAKAPβ peptide is at least a fragment of amino acids 2133-2319 of the mAKAPβ amino acid sequence or a nucleotide sequence having at least 80%, 85%, 90%, 95%, or 99% sequence identity thereto.

In another embodiment, the composition includes a small interfering RNA siRNA that inhibits the expression of any of RSK3, PP2A and mAKAPβ. In a preferred embodiment, the siRNA that inhibits the expression of mAKAPβ is generated in vivo following administration of a short hairpin RNA expression vector or biologic agent (shRNA).

The composition of the invention can be administered directly or can be administered using a viral vector. In a preferred embodiment, the vector is adeno-associated virus (AAV).

In another embodiment, the composition includes a small molecule inhibitor. In preferred embodiments, the small molecule is a RSK3, PP2A and/or mAKAPβ inhibitor.

In another embodiment, the composition includes a molecule that inhibits the binding, expression or activity of mAKAPβ. In a preferred embodiment, the molecule is a mAKAPβ peptide. The molecule may be expressed using a viral vector, including adeno-associated virus (AAV).

In yet another embodiment, the composition includes a molecule that interferes with mAKAPβ-mediated cellular processes. In some preferred embodiments, the molecule interferes with mAKAPβ binding to RSK3, or to the anchoring of PP2A.

The invention also relates to diagnostic assays for determining a propensity for heart disease, wherein the binding interaction of RSK3 and/or PP2A and mAKAPβ is measured, either directly, or by measuring a downstream effect of the binding of RSK3 and/or PP2A and mAKAPβ. The invention also provides a test kit for such an assay.

In still other embodiments, the inhibitors include any molecule that inhibits the expression of RSK3, PP2A and/or mAKAPβ, including antisense RNA, ribozymes and small interfering RNA (siRNA), including shRNA.

The invention also includes an assay system for screening of potential drugs effective to inhibit the expression and/or binding of RSK3 and/or PP2A and mAKAPβ. In one instance, the test drug could be administered to a cellular sample with the RSK3 and/or PP2A and mAKAPβ, or an extract containing the RSK3 and/or PP2A and mAKAPβ, to determine its effect upon the binding activity of the RSK3 and/or PP2A and mAKAPβ, by comparison with a control. The invention also provides a test kit for such an assay.

In preparing the peptide compositions of the invention, all or part of the RSK3 and/or PP2A or mAKAP amino acid sequence may be used. Preferably, at least 10 amino acids of the mAKAP sequence are used. More preferably, at least 25 amino acids of the mAKAP sequence are used. Most preferably, peptide segments from amino acids 1735-1833 or 2133-2319 of mAKAP are used.

It should be appreciated that various amino acid substitutions, deletions or insertions may also enhance the ability of the inhibiting peptide to inhibit the interaction of RSK3 and/or PP2A and mAKAPβ. A substitution mutation of this sort can be made to change an amino acid in the resulting protein in a non-conservative manner (i.e., by changing an amino acid belonging to a grouping of amino acids having a particular size or characteristic to an amino acid belonging to another grouping) or in a conservative manner (i.e., by changing an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes, which do not significantly alter the activity, or binding characteristics of the resulting protein.

The following is one example of various groupings of amino acids:

Amino acids with nonpolar R groups: Alanine, Valine, Leucine, Isoleucine, Proline, Phenylalanine, Tryptophan, Methionine.

Amino acids with uncharged polar R groups: Glycine, Serine, Threonine, Cysteine, Tyrosine, Asparagine, Glutamine.

Amino acids with charged polar R groups (negatively charged at pH 6.0): Aspartic acid, Glutamic acid.

Basic amino acids (positively charged at pH 6.0): Lysine, Arginine, Histidine.

Another grouping may be those amino acids with phenyl groups: Phenylalanine, Tryptophan, Tyrosine.

Another grouping may be according to molecular weight (i.e., size of R groups): Glycine (75), Alanine (89), Serine (105), Proline (115), Valine (117), Threonine (119), Cysteine (121), Leucine (131), Isoleucine (131), Asparagine (132), Aspartic acid (133), Glutamine (146), Lysine (146), Glutamic acid (147), Methionine (149), Histidine (at pH 6.0) (155), Phenylalanine (165), Arginine (174), Tyrosine (181), Tryptophan (204).

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure. Two amino acid sequences are “substantially homologous” when at least about 70% of the amino acid residues (preferably at least about 80%, and most preferably at least about 90 or 95%) are identical, or represent conservative substitutions.

Likewise, nucleotide sequences utilized in accordance with the invention can also be subjected to substitution, deletion or insertion. Where codons encoding a particular amino acid are degenerate, any codon which codes for a particular amino acid may be used. In addition, where it is desired to substitute one amino acid for another, one can modify the nucleotide sequence according to the known genetic code.

Nucleotides and oligonucleotides may also be modified. U.S. Pat. No. 7,807,816, which is incorporated by reference in its entirety, and particularly for its description of modified nucleotides and oligonucleotides, describes exemplary modifications.

Two nucleotide sequences are “substantially homologous” or “substantially identical” when at least about 70% of the nucleotides (preferably at least about 80%, and most preferably at least about 85%, 90%, 95% or 99%) are identical.

Two nucleotide sequences are “substantially complementary” when at least about 70% of the nucleotides (preferably at least about 80%, and most preferably at least about 85%, 90%, 95% or 99%) are able to hydrogen bond to a target sequence.

The term “standard hybridization conditions” refers to salt and temperature conditions substantially equivalent to 5×SSC and 65 C for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20 C below the predicted or determined Tₘ with washes of higher stringency, if desired.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30%, 40%, 50%, 60%, 70%, 80% or 90% a clinically significant change in a cardiac myocyte feature.

The preparation of therapeutic compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment, as well as a small molecule inhibitor, can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic compositions of the invention are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition of RSK3 and/or PP2A-mAKAPβ binding desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated. In one preferred embodiment, mAKAPβ peptides or shRNA are expressed by an AAV gene therapy vector. Suitable intravenous doses for AAV vectors range from 1×10¹² viral genomes/kilogram body weight to 5×10¹⁴ viral genomes/kilogram body weight. In particular, for AAV vectors containing a serotype 9 capsid, doses are preferably 0.3-1×10¹⁴ viral genomes/kilogram body weight.

Because of the necessity for the inhibitor to reach the cytosol, a peptide in accordance with the invention may need to be modified in order to allow its transfer across cell membranes, or may need to be expressed by a vector which encodes the peptide inhibitor. Likewise, a nucleic acid inhibitor (including siRNAs, shRNAs and antisense RNAs) can be expressed by a vector. Any vector capable of entering the cells to be targeted may be used in accordance with the invention. In particular, viral vectors are able to “infect” the cell and express the desired RNA or peptide. Any viral vector capable of “infecting” the cell may be used. A particularly preferred viral vector is adeno-associated virus (AAV).

siRNAs inhibit translation of target mRNAs via a process called RNA interference. When the siRNA is perfectly complementary to the target mRNA, siRNA act by promoting mRNA degradation. shRNAs, as a specialized type of siRNA, have certain advantages over siRNAs that are produced as oligonucleotides. siRNA oligonucleotides are typically synthesized in the laboratory and are delivered to the cell using delivery systems that deliver the siRNA to the cytoplasm. In contrast, shRNAs are expressed as minigenes delivered via vectors to the cell nucleus, where following transcription, the shRNA are processed by cellular enzymes such as Drosha and Dicer into mature siRNA species. siRNAs are usually 99% degraded after 48 hours, while shRNAs can be expressed up to 3 years or longer. Moreover, shRNAs can be delivered in much lower copy number than siRNA (5 copies vs. low nM), and are much less likely to produce off-target effects, immune activation, inflammation and toxicity. While siRNAs are suitable for acute disease conditions where high doses are tolerable, shRNAs are suitable for chronic, life threatening diseases or disorders where low doses are desired. (http://www.benitec.com/technology/sirna-vs-shrna)

Guidelines for the design of siRNAs and shRNAs can be found in (Elbashir et al. 2001) and at various websites including https://www.thermofisher.com/us/en/home/references/ambion-tech-support/rnai-sirna/general-articles/-sirna-design-guidelines.html and http://www.invivogen.com/review-sirna-shrna-design, all of which are hereby incorporated by reference in their entireties. Preferably, the first nucleotide is an A or a G. siRNAs of 25-29 nucleotides may be more effective than shorter ones, but shRNAs with duplex length 19-21 seem to be as effective as longer ones. siRNAs and shRNAs are preferably 19-29 nucleotides. Loop sequences in shRNAs may be 3-9 nucleotides in length, with 5, 7 or 9 nucleotides preferred.

Exemplary shRNA sequences of the invention include GGTTGAAGCTTTGAAGAAA (SEQ ID NO:77), GCTAAGAGATACAGAGCTT (SEQ ID NO:78) or GGAGGAAATAGCAAGGTTA (SEQ ID NO:79).

With respect to small molecule inhibitors, any small molecule that inhibits the interaction of RSK3 and/or PP2A and mAKAPβ may be used. In addition, any small molecules that inhibit the activity of RSK3 and/or PP2A and/or mAKAPβ may be used.

Small molecules with similar structures and functionalities can likewise be determined by rational and screening approaches.

Likewise, any small molecules that inhibit the expression of RSK3, PP2A and/or mAKAPβ may be used.

In yet more detail, the present invention is described by the following items which represent preferred embodiments thereof:

1. A composition comprising a regulatory nucleotide sequence for expression of a second nucleotide sequence in a cardiac myocyte, wherein said regulatory nucleotide sequence comprises an intronic sequence comprising a splicing consensus site, wherein said intronic sequence is from the human cardiac troponin T gene. (hTNNT).

2. The composition of Item 1, further comprising a TNNT2 promoter sequence.

3. The composition of Item 1, wherein the regulatory nucleotide sequence is in a vector.

4. The composition of Item 3, further comprising a transgene.

5. The composition of Item 4, wherein the transgene is a muscle A-kinase anchoring protein β (mAKAPβ) sequence.

6. The composition of Item 5, wherein the mAKAPβ sequence is an shRNA.

7. The composition of Item 6, wherein the shRNA comprises GGTTGAAGCTTTGAAGAAA (SEQ ID NO: 77), GCTAAGAGATACAGAGCTT (SEQ ID NO: 78) or GGAGGAAATAGCAAGGTTA (SEQ ID NO: 79).

8. The composition of Item 3, wherein the vector encodes an amino acid sequence having at least 80% sequence homology to a fragment of mAKAPβ.

9. The composition of Item 8, wherein the vector encodes an amino acid sequence having at least 90% sequence identity to a fragment of mAKAPβ.

10. The composition of Item 9, wherein the amino acid sequence encodes a fragment of mAKAPβ.

11. The composition of Item 8, wherein the amino acid sequence binds a kinase.

12. The composition of Item 11, wherein the kinase is p90 ribosomal S6 kinase 3 (RSK3).

13. The composition of Item 12, wherein amino acid sequence inhibits the binding of mAKAPβ to RSK3.

14. The composition of Item 10, wherein the amino acid sequence has at least 80% sequence homology to amino acids 1694-1757, 1735-1833 or 1694-1833 of mAKAP.

15. The composition of Item 14, wherein the amino acid sequence has at least 90% sequence identity to amino acids 1735-1833 of mAKAP.

16. The composition of Item 12, wherein the amino acid sequence comprises a RSK3 binding domain (RBD) of mAKAPβ.

17. The composition of Item 15, wherein the RBD comprises amino acids 1735-1833 of SEQ ID NO:12.

18. The composition of Item 11, wherein the amino acid sequence binds protein phosphatase 2A (PP2A).

19. The composition of Item 18, wherein amino acid sequence inhibits the anchoring PP2A to mAKAPβ.

20. The composition of Item 19, wherein the amino acid sequence has at least 80% sequence homology to amino acids 2132-2319 of mAKAP.

22. The composition of Item 20, wherein the amino acid sequence has at least 90% sequence identity to amino acids 2132-2319 of mAKAP.

23. The composition of Item 20, wherein the amino acid sequence comprises a PP2A binding domain (PBD) of mAKAPβ.

24. The composition of Item 23, wherein the PBD comprises amino acids 2132-2319 of SEQ ID NO:12.

25. The composition of any one of Item 11, 14 or 15, wherein the kinase is Ca²⁺/calmodulin-dependent protein kinase II (CaMKII).

26. The composition of any one of Items 3-25, wherein the vector is adeno-associated virus (AAV).

27. The composition of any one of Items 3-26, wherein the vector further comprises SV40 polyadenylation sequences.

28. The composition of Item 5, wherein human mAKAP amino acids 2132-2319 (SEQ ID NO:11) has been modified at one or more of the following positions: TCG at amino acid 2144 has been modified to TCA; AGC at amino acid 2183 has been modified to AGT; TCC at amino acid 2256 has been modified to TCA; GCC at amino acid 2291 has been modified to GCA; or CGA at amino acid 2313 has been modified to AGA.

29. The composition of Item 5, wherein human mAKAP amino acids 1696-1835 (SEQ ID NO:11) encoding RBD has been modified at one or more of the following positions: CCG at amino acid 1712 has been modified to CCA; TCG at amino acid 1714 has been modified to TCT; TCG at amino acid 1717 has been modified to TCT; CGT at amino acid 1721 has been modified to AGA; CGT at amino acid 1724 has been modified to AGA; AGC at amino acid 1730 has been modified to AGT; AGC at amino acid 1753 has been modified to AGT; and GAC at amino acid 1775 has been modified to GAT.

30. A method of treating or preventing heart disease, comprising administering to cardiac cells of a patient a vector of any one of Items 1-29.

31. A method of treating or preventing heart disease, comprising administering to cardiac cells of a patient a vector of Item 6 or Item 7, wherein the method inhibits the expression of mAKAP.

Several recent clinical trials have reported varying degrees of immunotoxicity followed by administration of recombinant adeno-associated viruses (rAAV), thus limiting the durability and success of gene therapies in humans (Wright 2020; Hamilton and Wright 2021). Molecules such as unmethylated CpG dinucleotide-based motifs (CpGs) signal AAV viral infection (Akira, Uematsu, and Takeuchi 2006; Kanneganti, Lamkanfi, and Nunez 2007) and promote host innate immune responses via activation of the Toll-like receptor TLR9-MyD88 signaling pathway, which results in recruitment of cytotoxic T lymphocytes (CTL) onto infected cells (Hartmann, Weiner, and Krieg 1999; Ohto et al. 2018; Zhu, Huang, and Yang 2009; Shirley et al. 2020; Xiang et al. 2020). Furthermore, vaccine research using oligonucleotides as adjuvants has informed the scientific community of CpG-containing immunostimulatory (e.g. ACGT, TCGT, CCGT) and -inhibitory (e.g. GCGG, CCGC, GCGC) motifs (Wright 2020; Ohto et al. 2015; Bode et al. 2011; Pohar et al. 2017).

Due to differences in sensitivity of TLR/innate receptors in humans vs most other pre-clinical animal models (Tahtinen et al. 2022; Hawash et al. 2021), immunostimulatory features of AAV gene therapies in human patients might not manifest themselves during pre-clinical development and are not expected in rodent or swine studies. However, to prevent potential immunologic reactions and loss of transgene expression in patients, new versions of mAKAPβ-targeting AAV biologics have been designed. The newly proposed viral genome (vg) configurations feature new expression cassettes conceived for enhanced cardiac expression and reduced immunogenicity in humans. Sequences have been optimized by removing CpG and CpG-containing immunostimulatory motifs, while retaining CpG-containing immune-inhibitory motifs.

Off-target effects can also be inhibited by the use of tissue-specific promoters that limit the expression of the gene of interest to relevant cell types. The TNNT2 gene promoter has been used as a means for cardiac myocyte-selective expression of recombinant proteins and shRNA (Prasad et al. 2011; Martinez et al. 2022; Li et al. 2020) (U.S. Pat. No. 11,129,908 B2). The present constructs include a new configuration of the human TNNT2 promoter that includes additional promoter sequence, sequence from exons 1 and 2 and the first intron that confers greater cardiac myocyte-specificity in expression and that is useful for in vivo applications. This new TNNT2 promoter composition is useful not only for AAV gene therapies, but also for transgenesis and transient expression in cardiac myocyte specific applications based upon both plasmid and viral vectors.

The following examples are provided to aid the understanding of the present invention, the true scope of which is set forth in the appended claims. It is understood that modifications can be made in the procedures set forth without departing from the spirit of the invention.

### EXAMPLES

The compositions and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the processes, formulations and/or methods of the invention may be made without departing from the spirit of the invention and the scope of the appended claims.

### Example 1

### New AAV Compositions

For each of the three AAV9 self-complementary biologics shown here, two alternative layouts are presented, representing alternative compositions for cardiac myocyte-specific gene therapies:

1—Human cardiac troponin T 2 Regulatory Sequences that contains promoter, exonic and intronic sequences that include a skeletal muscle enhancer and splicing consensus site (hTNNT+enh+int)+transgene+SV40 poly adenylation (SV40polyA).

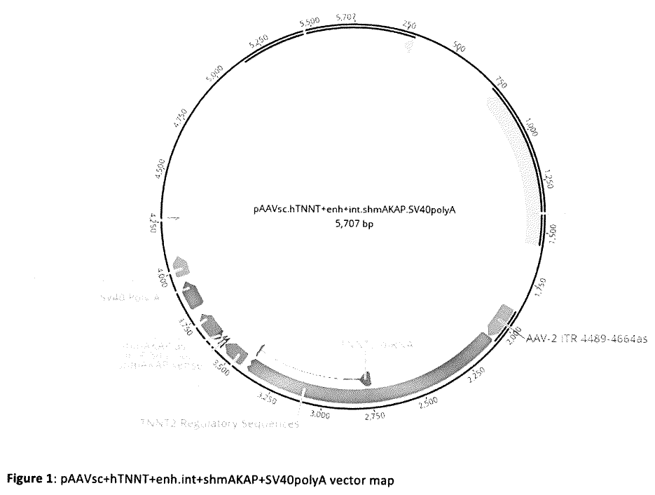

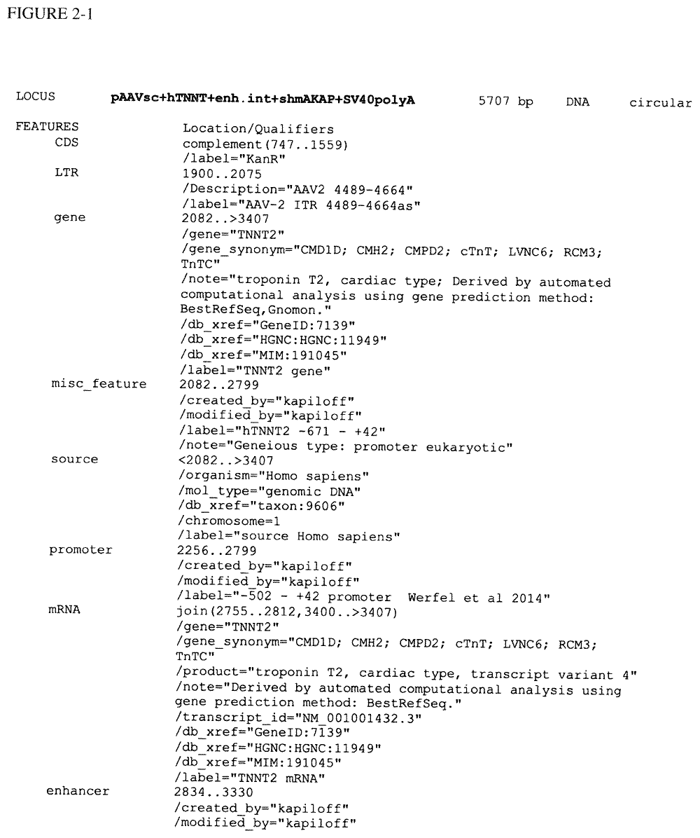

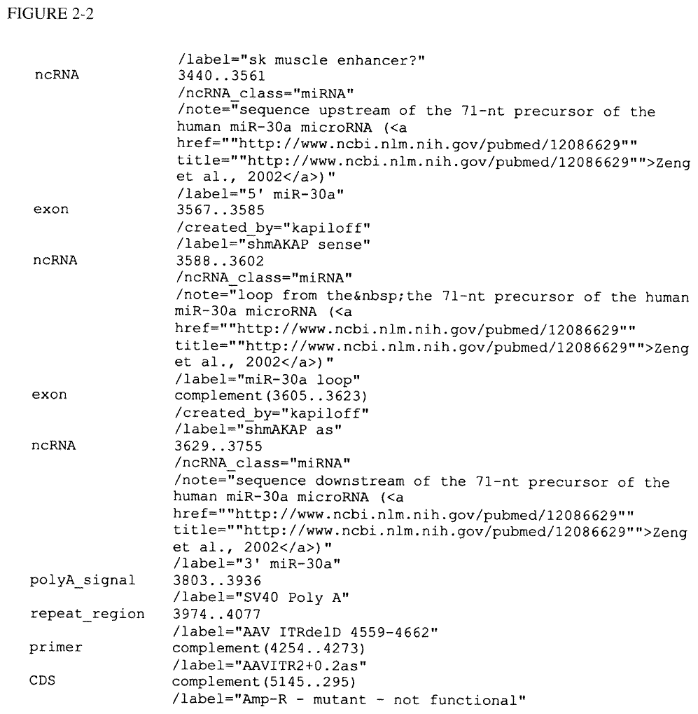

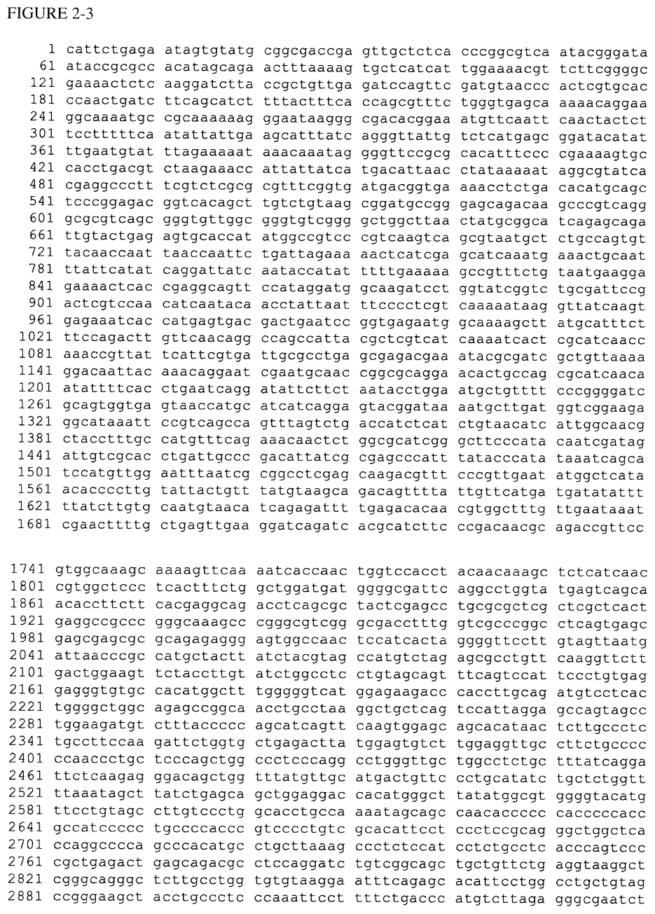

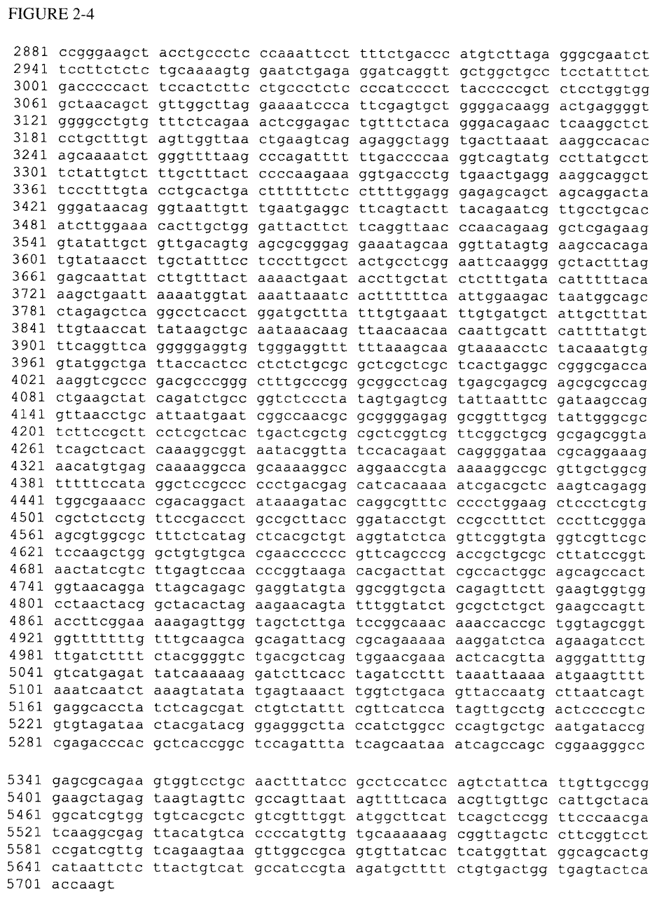

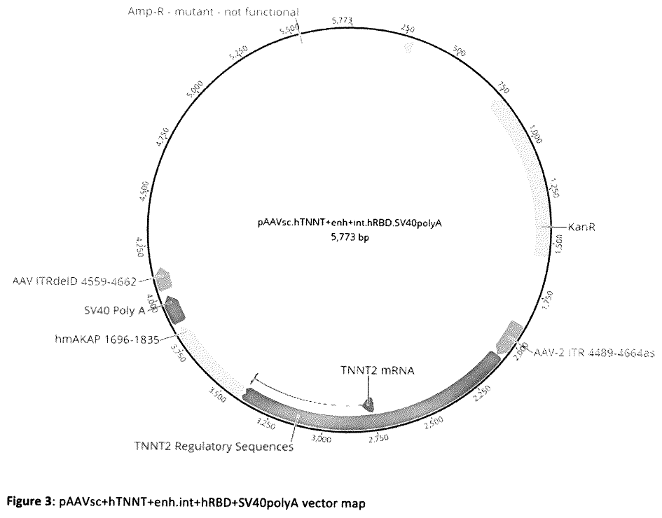

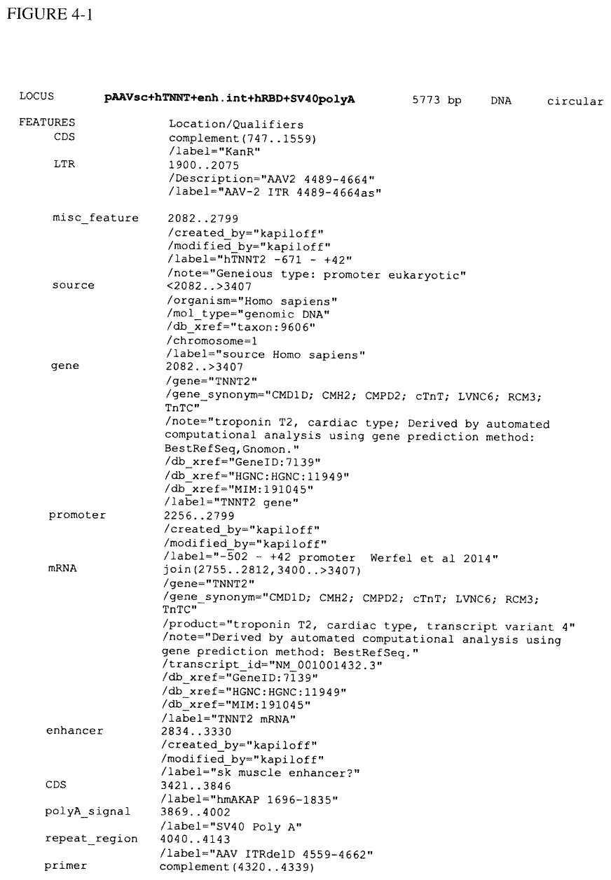

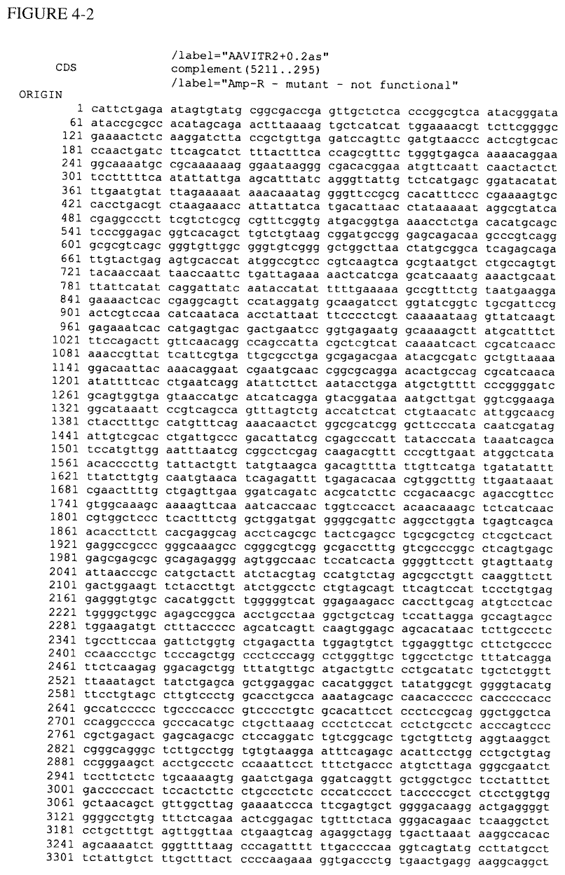

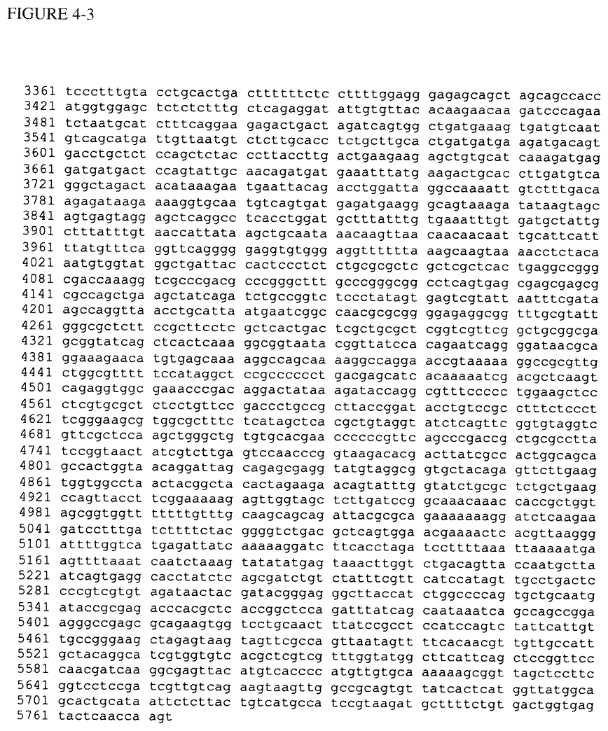

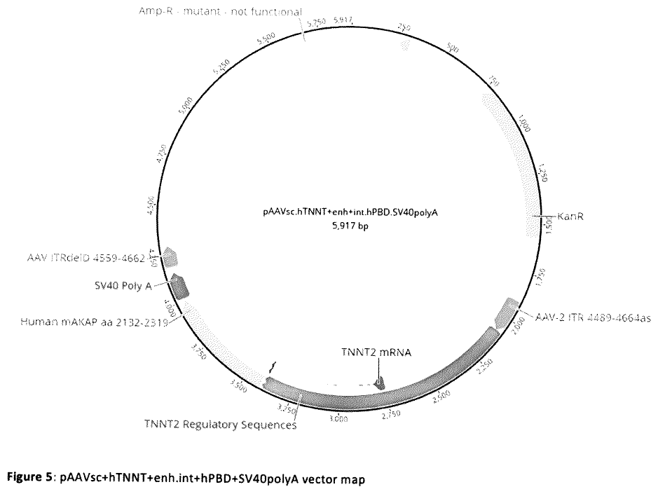

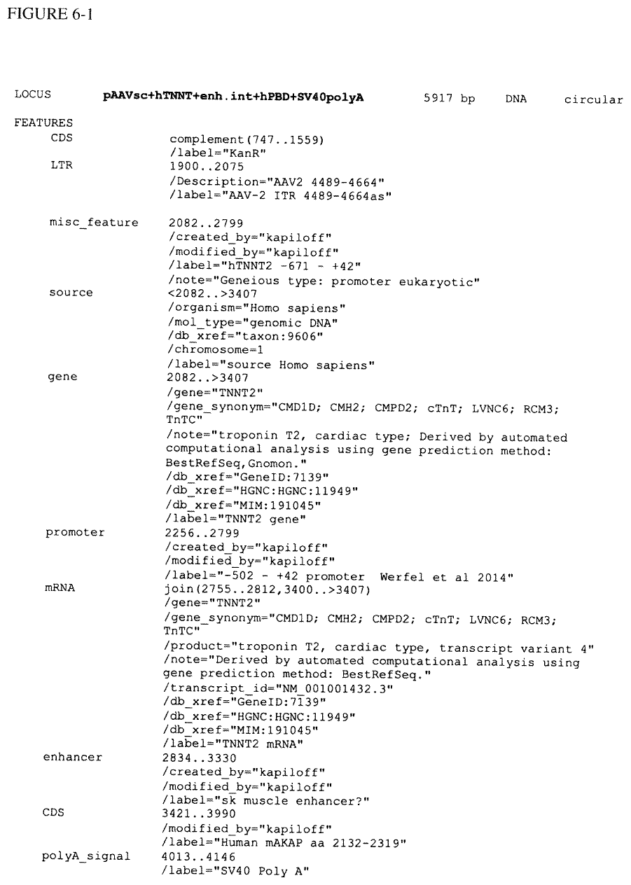

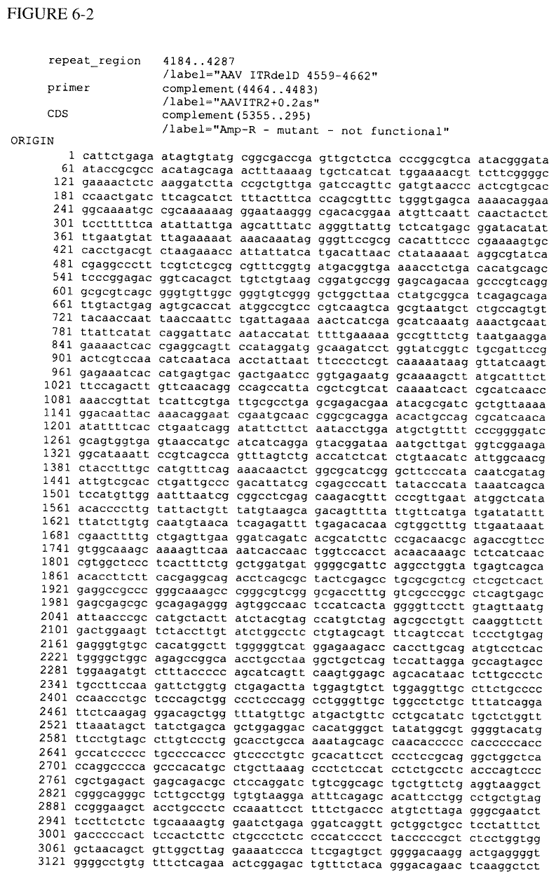

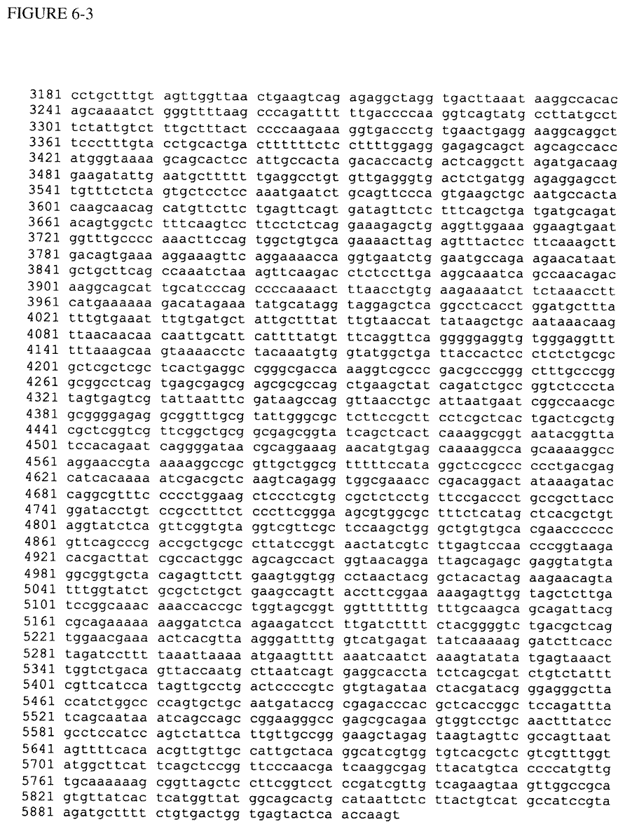

Complete plasmid sequences and corresponding vector maps are shown in FIGS. 1-6.
Composition #1: pAAVsc.hTNNT+enh.int.shmAKAP.SV40polyA,
Composition #2: pAAVsc.hTNNT+enh.int.hRBD.SV40polyA,
Composition #3: pAAVsc.hTNNT+enh.int.hPBD.SV40polyA.

2—Human calsequestrin enhancer (calseq)+human cardiac troponin T 2 promoter (hTNNT)+SV40 16S synthetic intron (SV40int.)+transgene+SV40 poly adenylation.

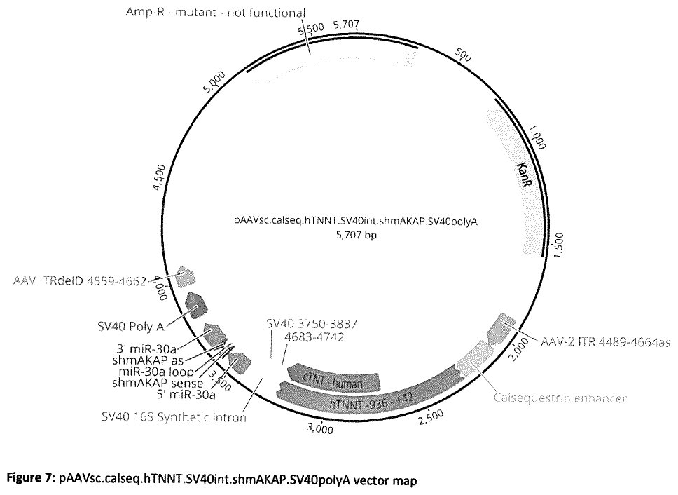

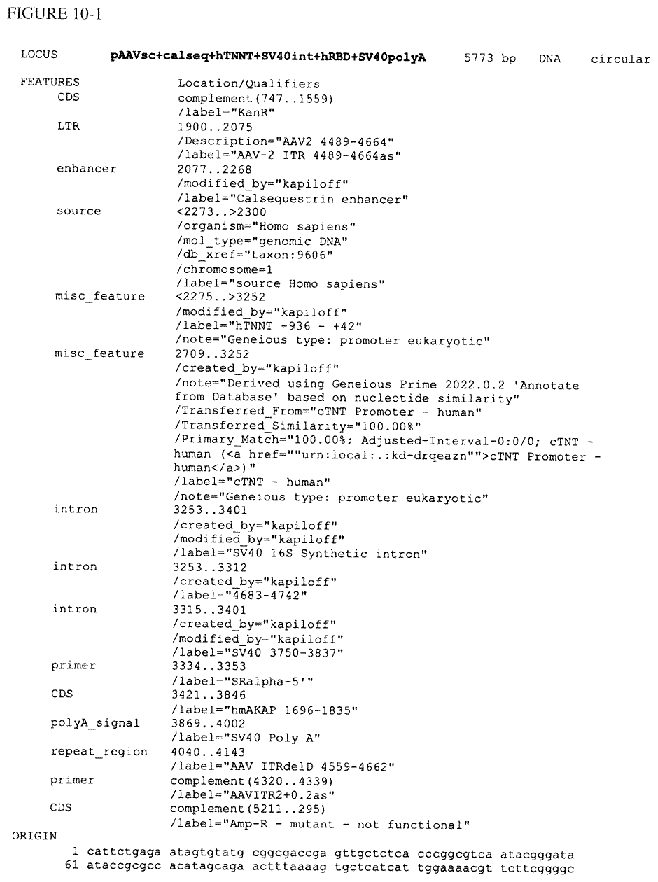

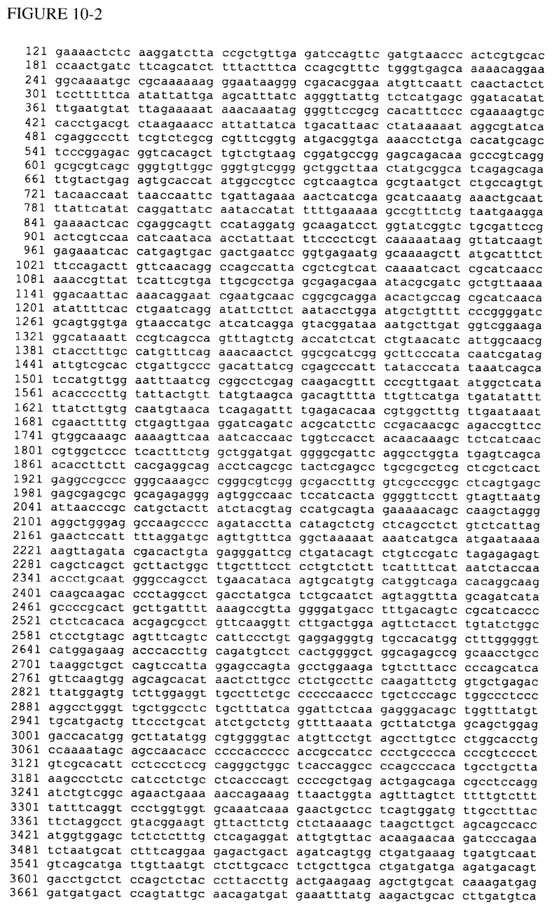

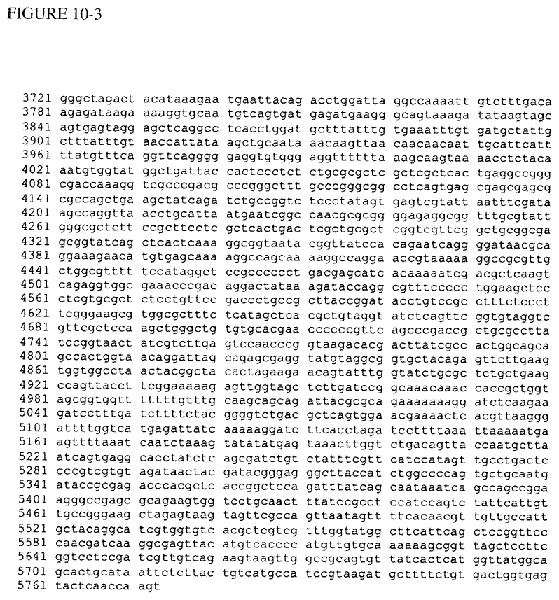

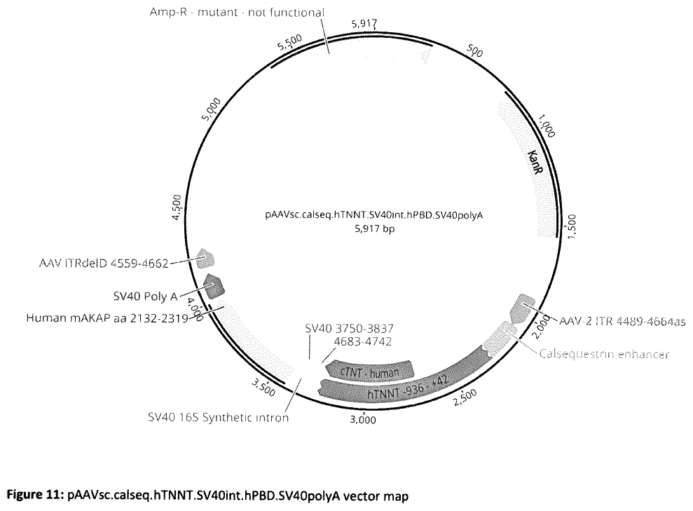

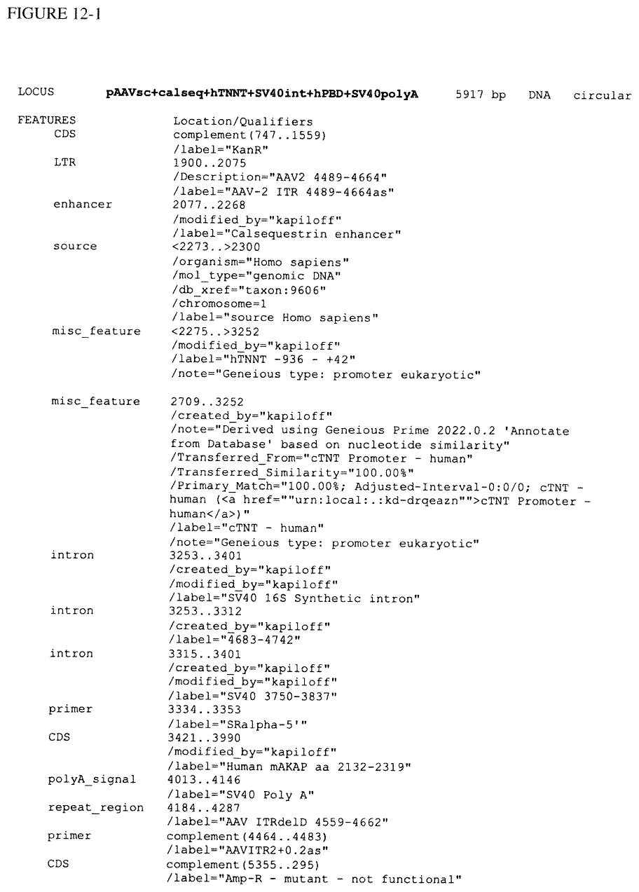

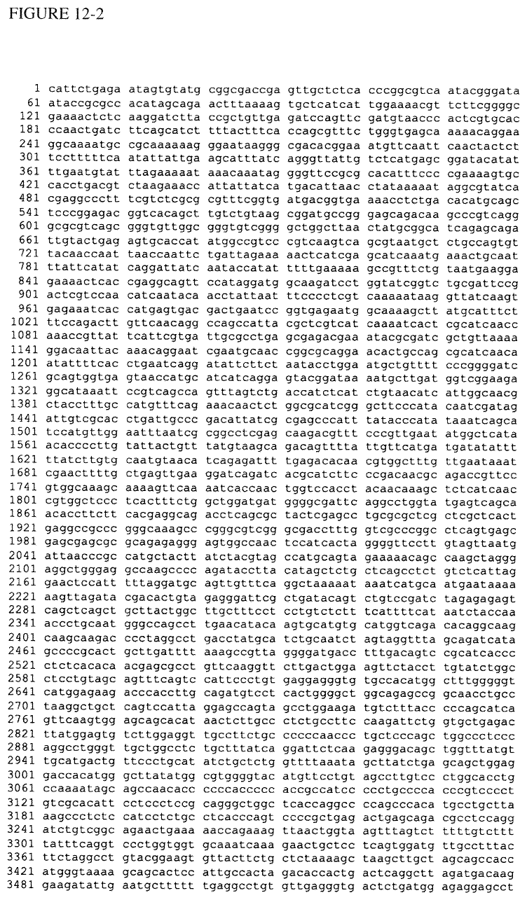

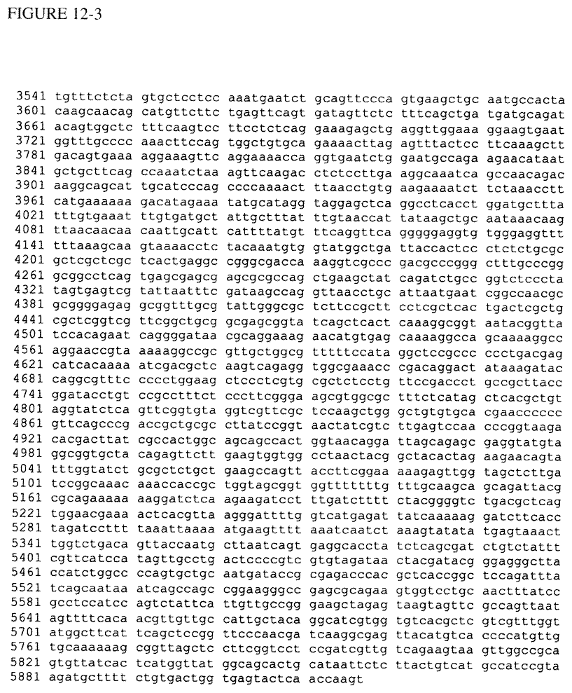

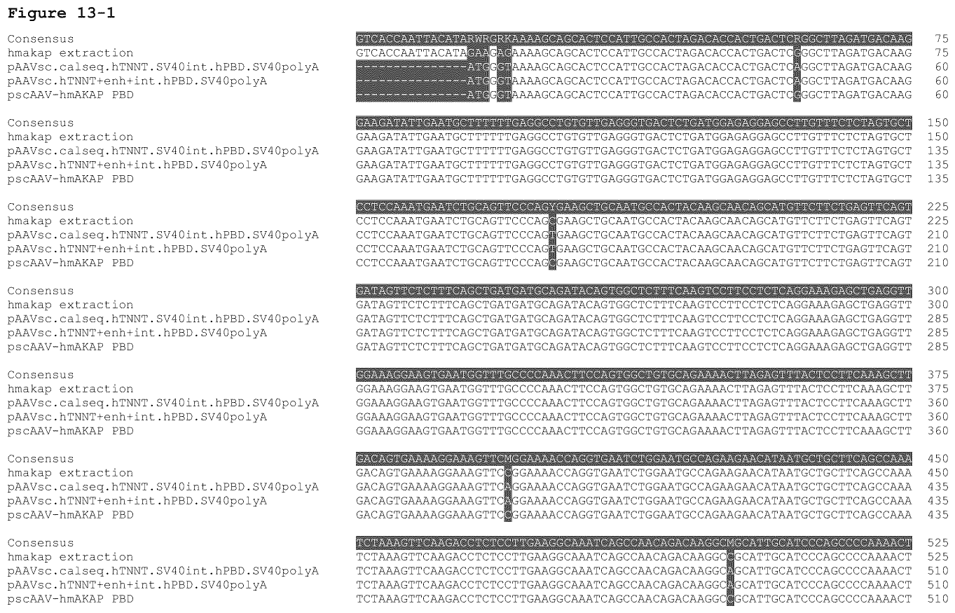

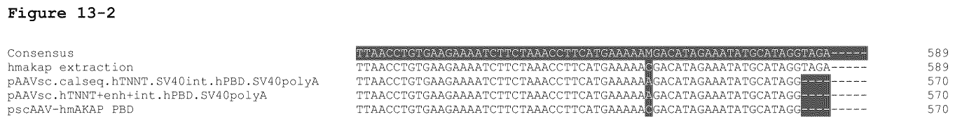

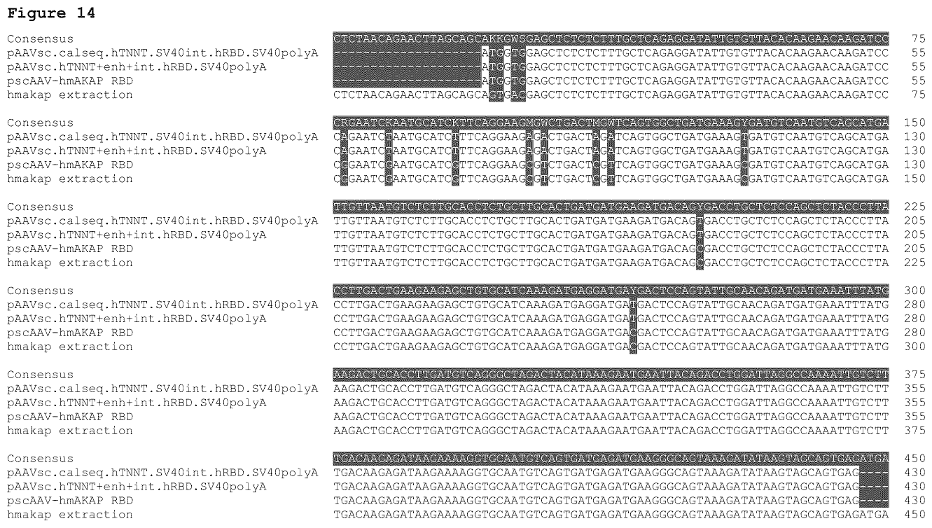

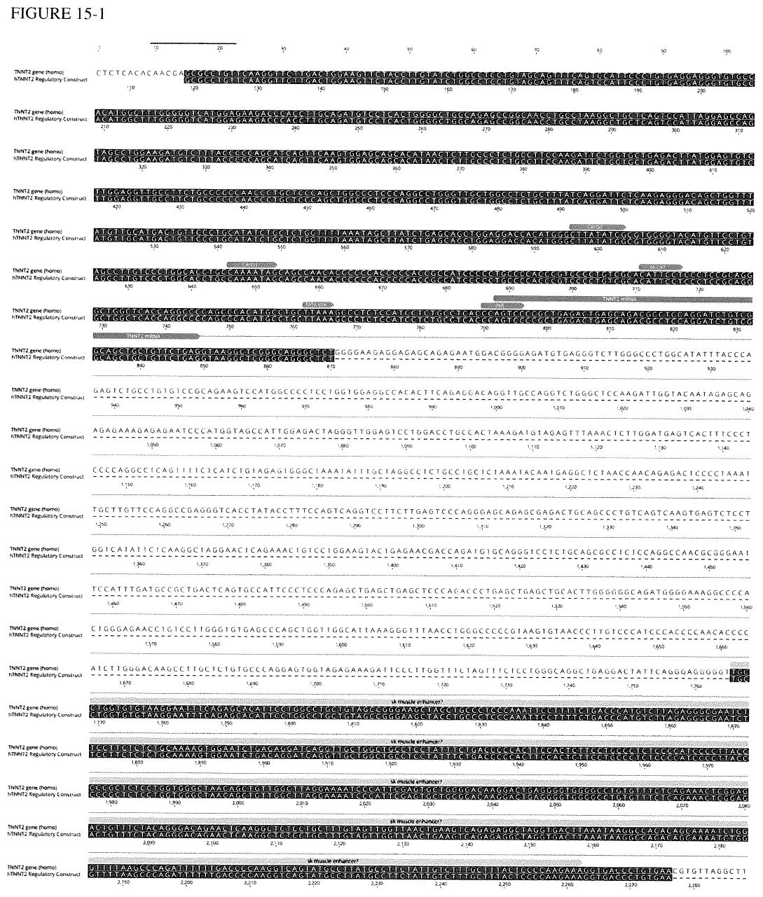

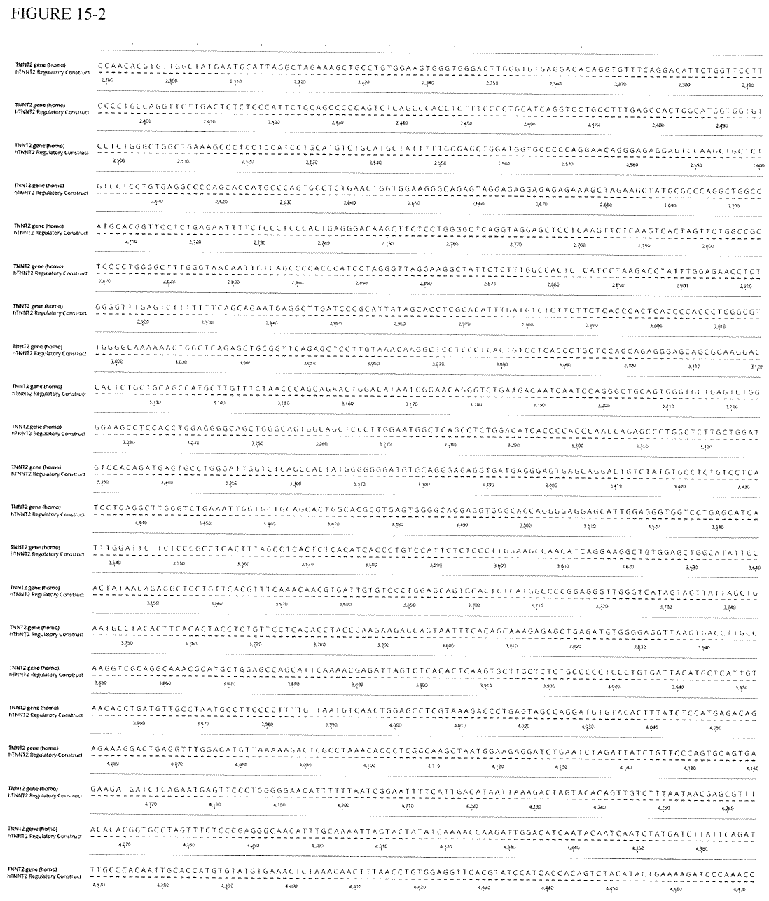

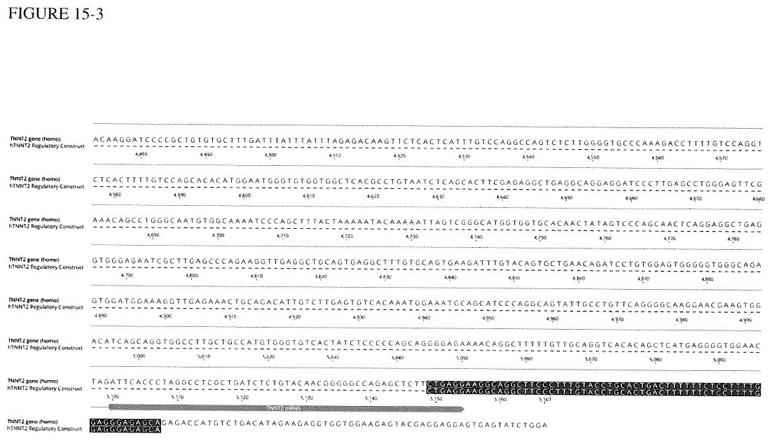

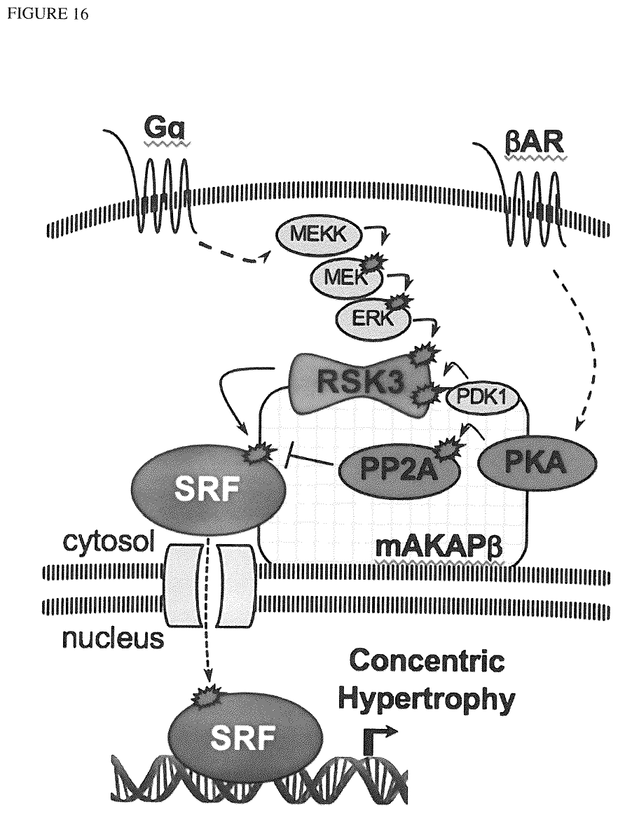

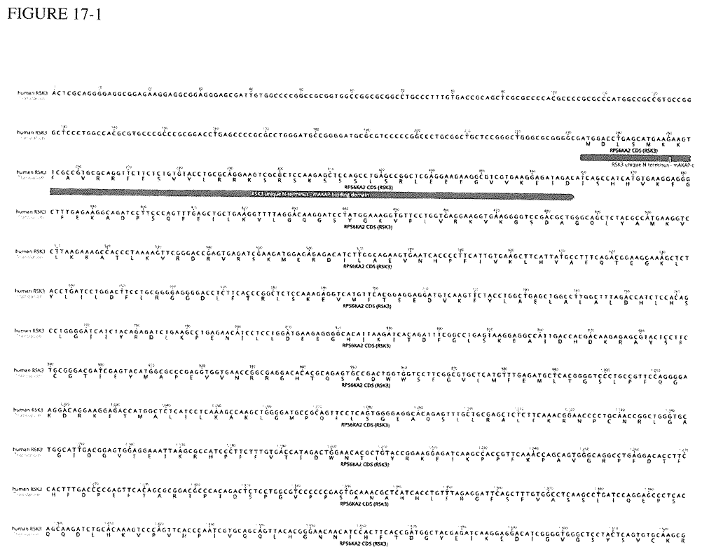

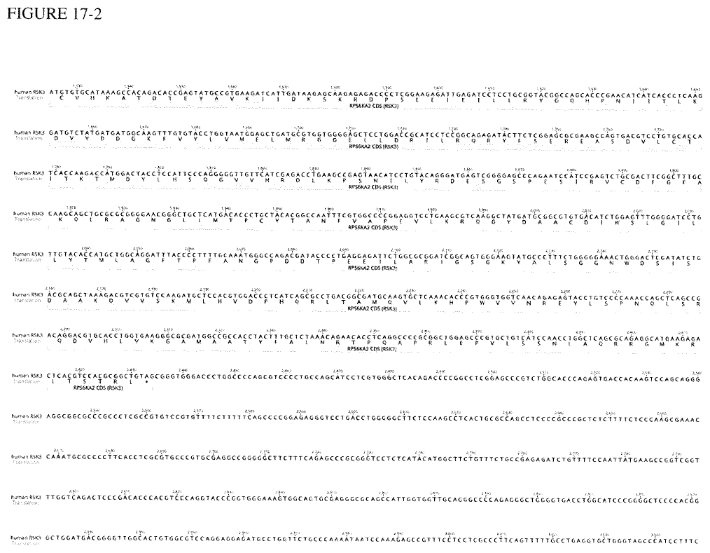

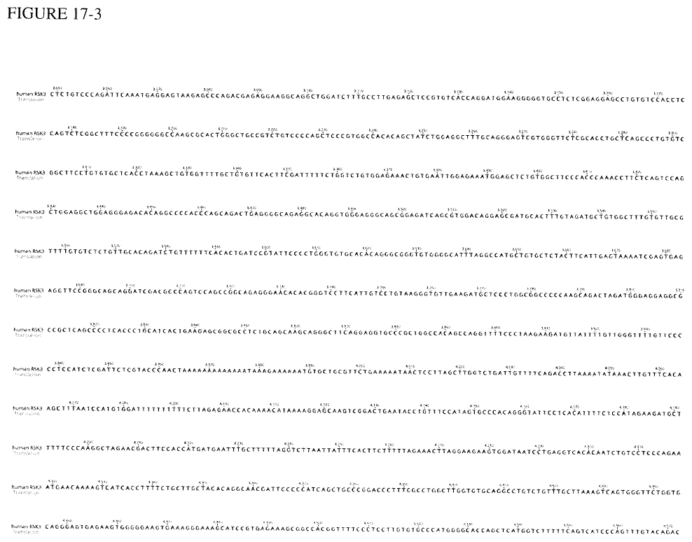

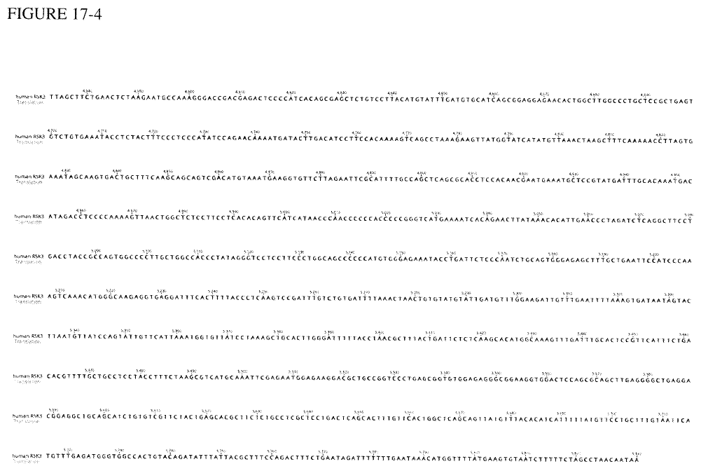

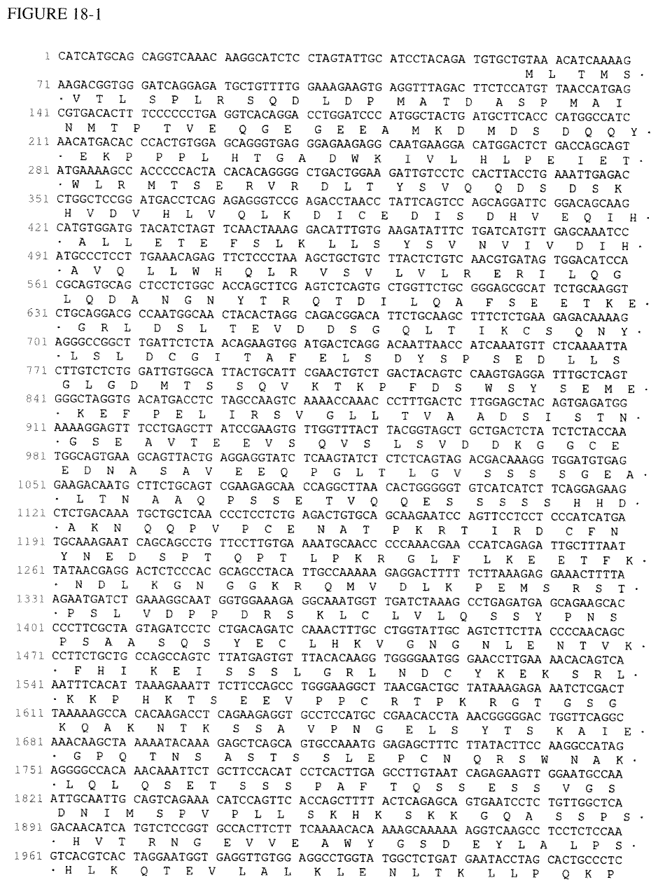

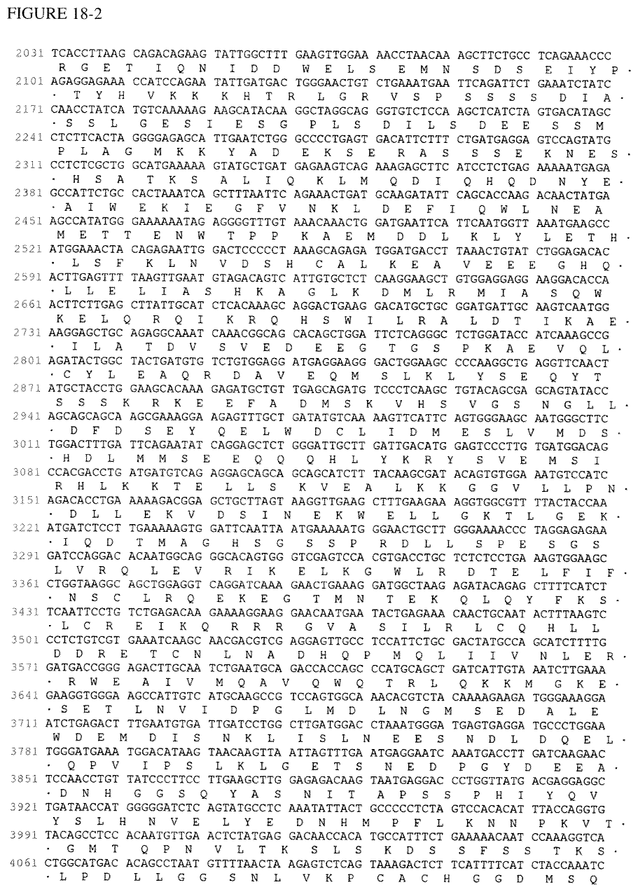

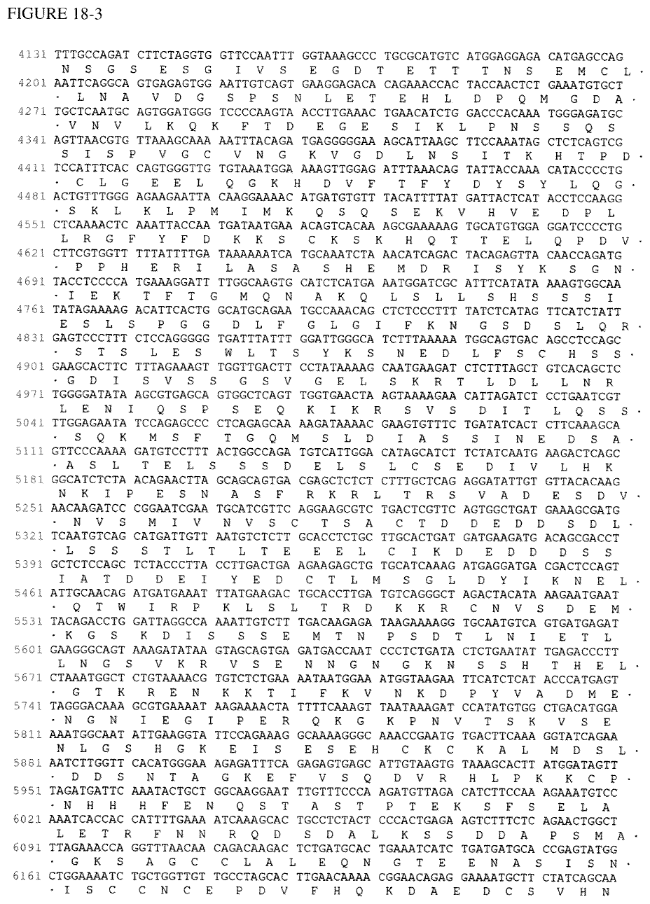

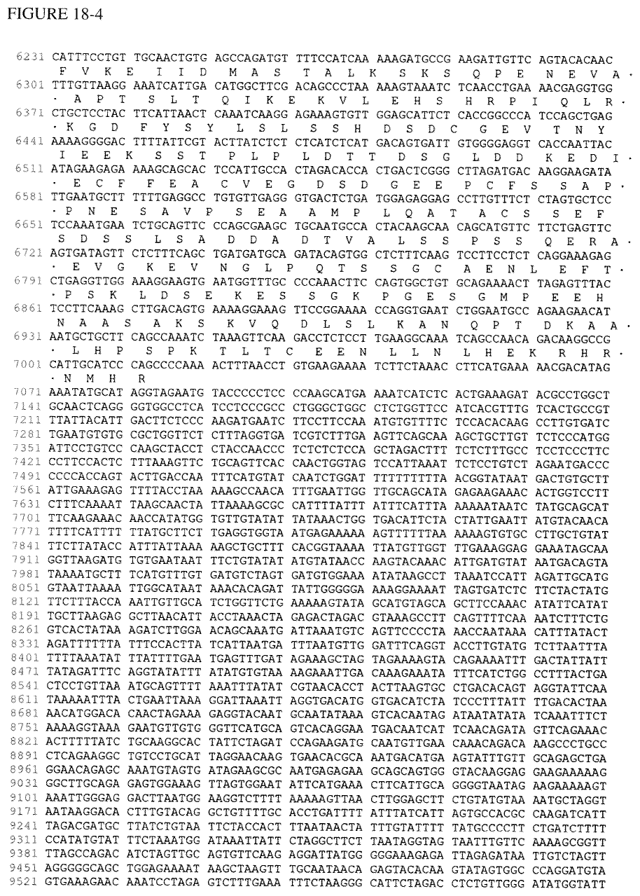

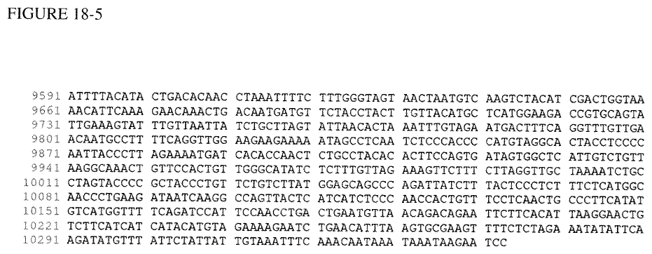

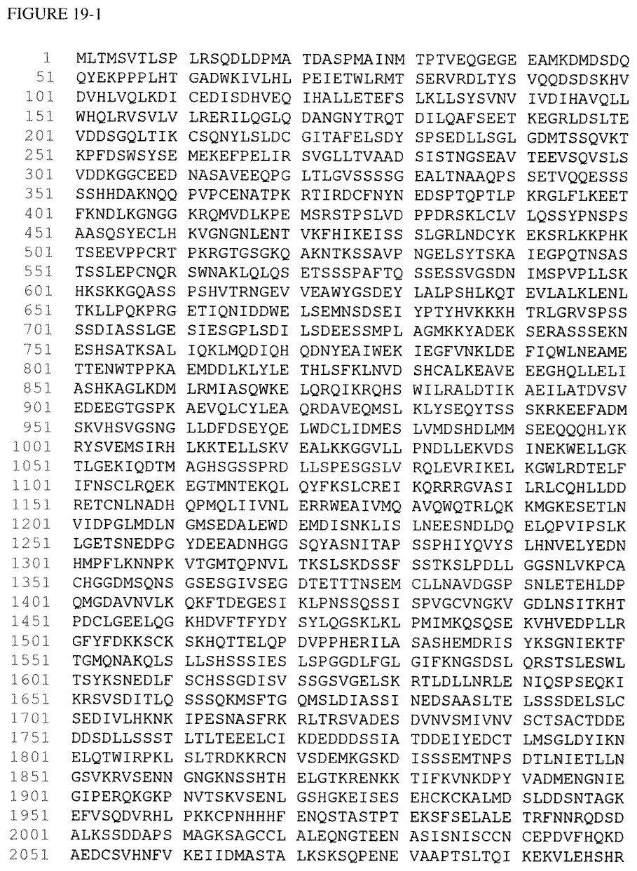

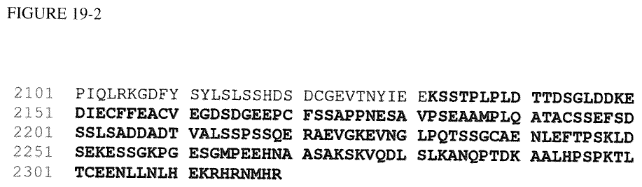

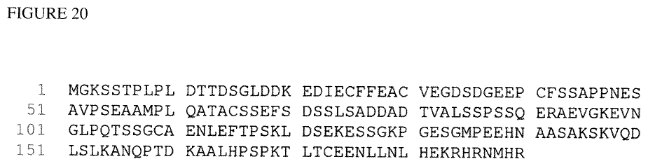

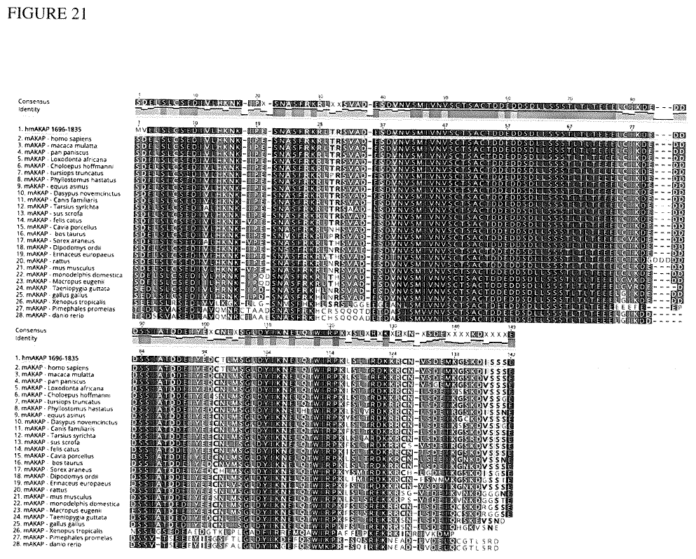

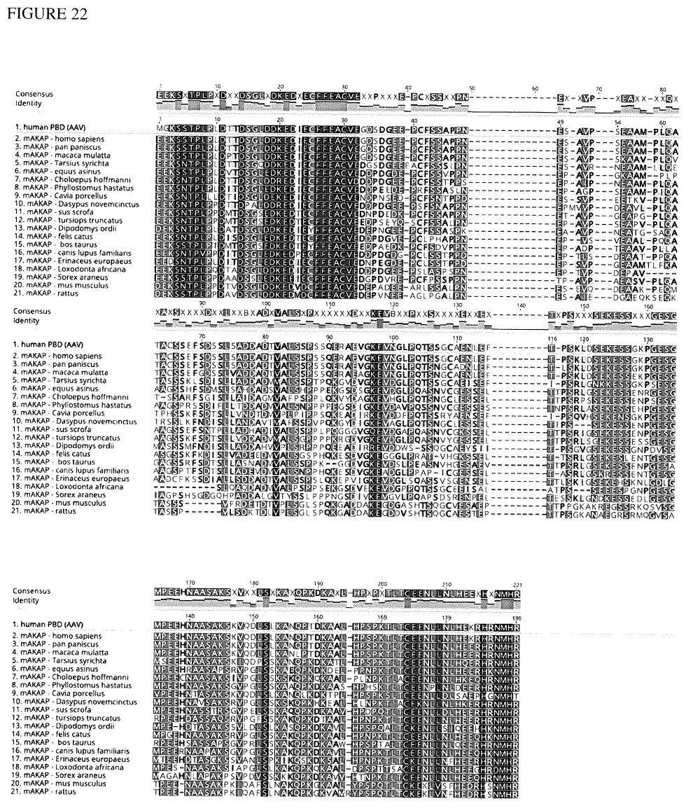

Complete plasmid sequences and corresponding vector maps are shown in FIGS. 7-12.
Composition #4: pAAVsc.calseq.hTNNT.SV40int.shmAKAP.SV40polyA,
Composition #5: pAAVsc.calseq.hTNNT.SV40int.hRBD.SV40polyA,
Composition #6: pAAVsc.calseq.hTNNT.SV40int.hPBD.SV40polyA.

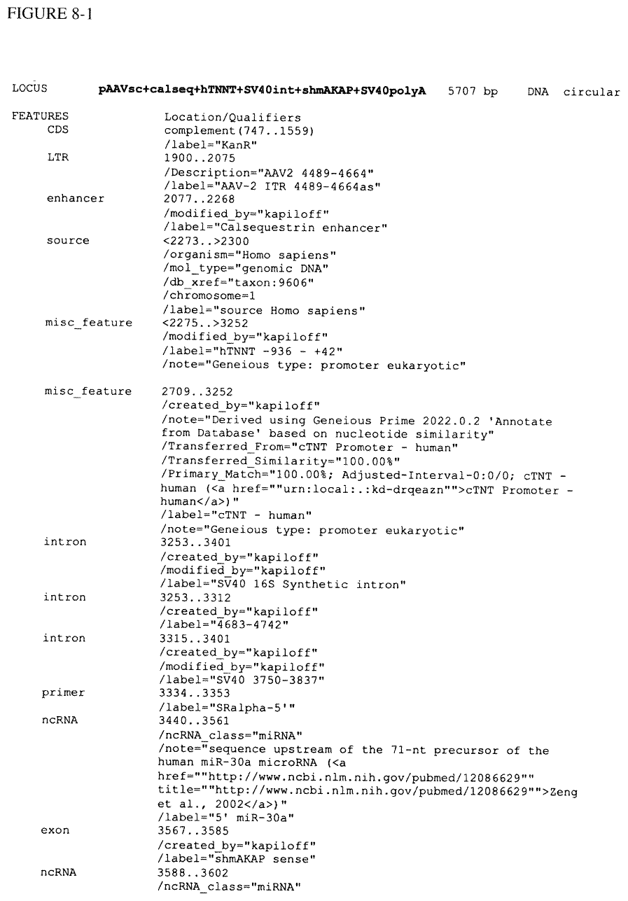

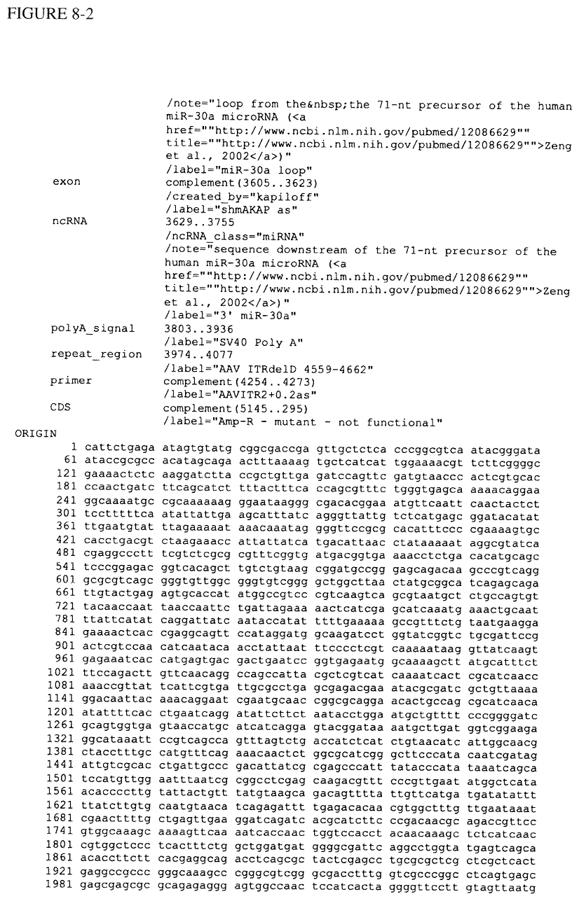

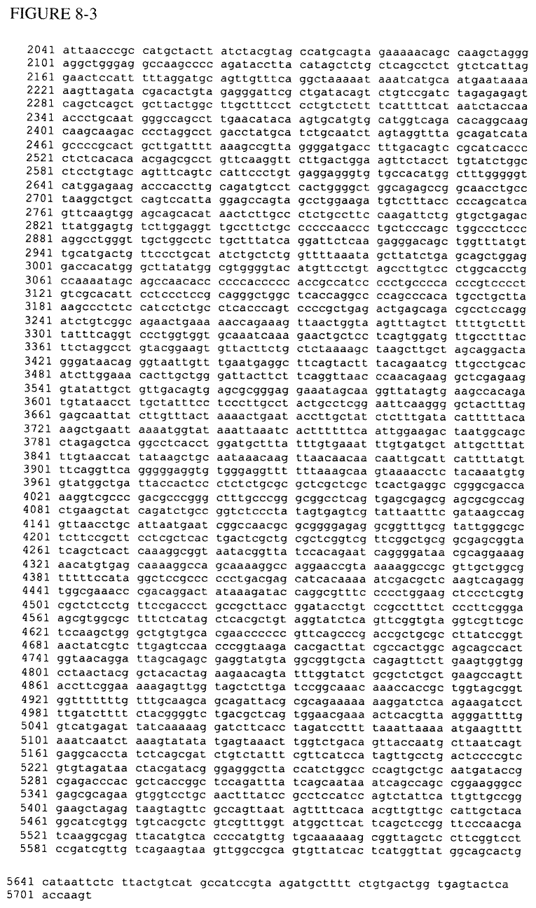

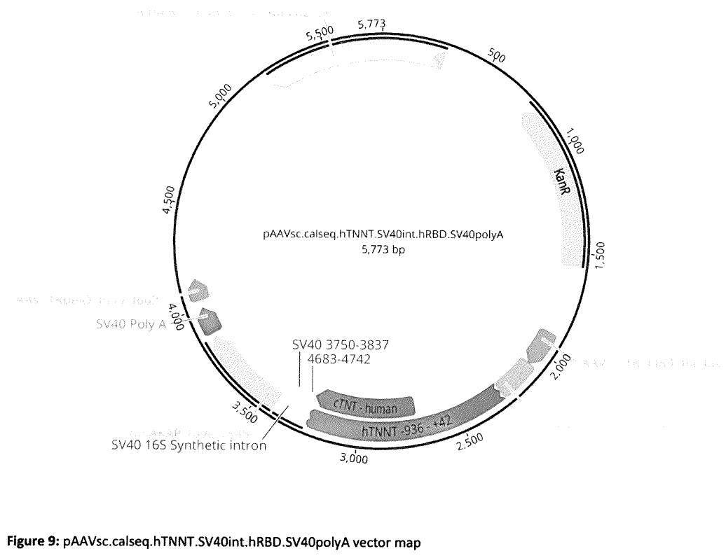

All elements were optimized for CpG content by selecting human genomic sequences displaying the least amount of CpG dinucleotides as well as CpG-containing tetranucleotides known to stimulate immune reactions. Also, codon optimization was performed, where possible, to remove inflammatory motifs from open reading frame (ORF) sequences. Sequence alignments are displayed in FIGS. 8 and 9. A detailed list of modifications summarized per ORF is herein provided:

| | |
|---|---|
| Human mAKAP aa 2132-2319 (PBD): | Human mAKAP aa 1696-1835 (RBD): |
| aa S2144_TCG > TCA | aa P1712_CCG > CCA |
| aa S2183_AGC > AGT | aa S1714_TCG > TCT |
| aa S2256_TCC > TCA | aa S1717_TCG > TCT |
| aa A2291_GCC > GCA | aa R1721_CGT > AGA |
| aa R2313_CGA > AGA | aa R1724_CGT > AGA |
| | aa S1730_AGC > AGT |
| | aa S1753_AGC > AGT |
| | aa D1775_GAC > GAT |

### Compositions 1-3

In the first proposed configuration, cardiac specific expression is achieved with a novel combination of regulatory elements. The novel TNNT2 transcription regulatory sequences includes: 1) −673 to +79 TNNT2 promoter, Exon 1, and adjacent Intron 1 sequence (relative to transcript_id: NM_000364.4; NCBI chromosome reference: NC_000001.11 201,378,353..201,377,602, bp 1-752 of novel construct “New regulatory”); 2) an intronic enhancer (NC_000001.11 201,376,706..201,376,196, bp 753-1263 of novel construct); and 3) Intron 1 3′ sequence and Exon 2 partial sequence (NC_000001.11 201,373,323..201,373,261, bp 1264-1326 of novel construct).

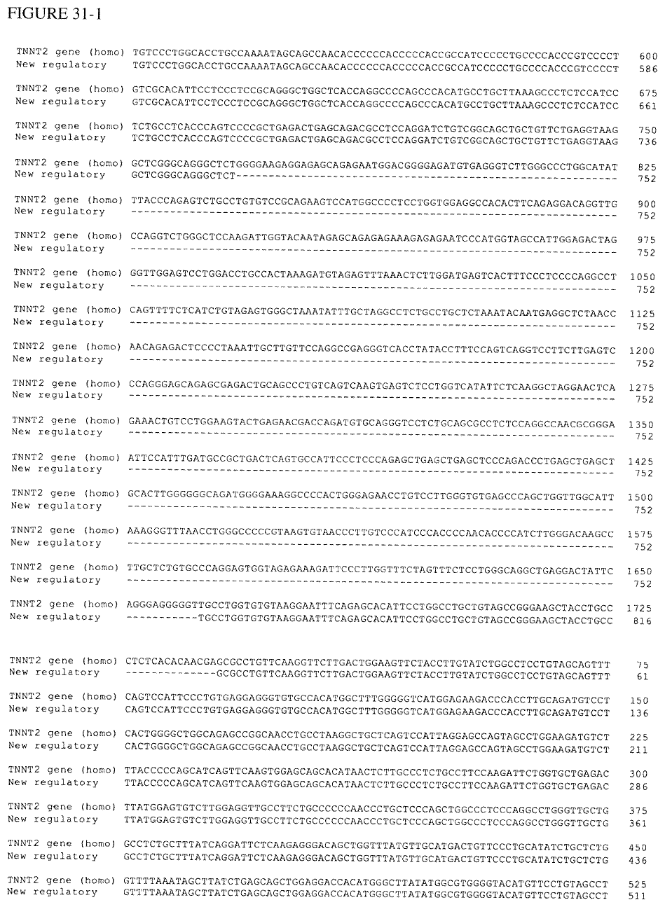

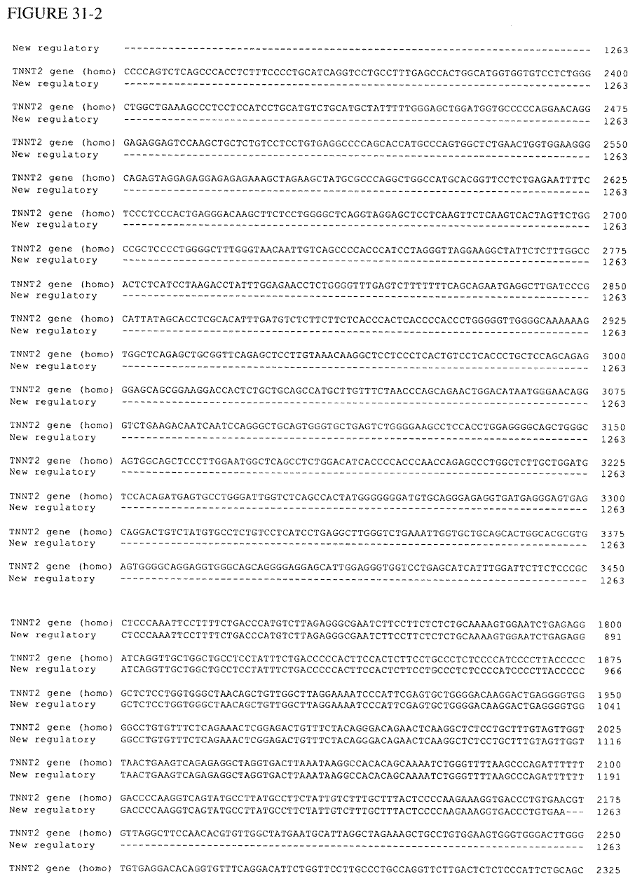

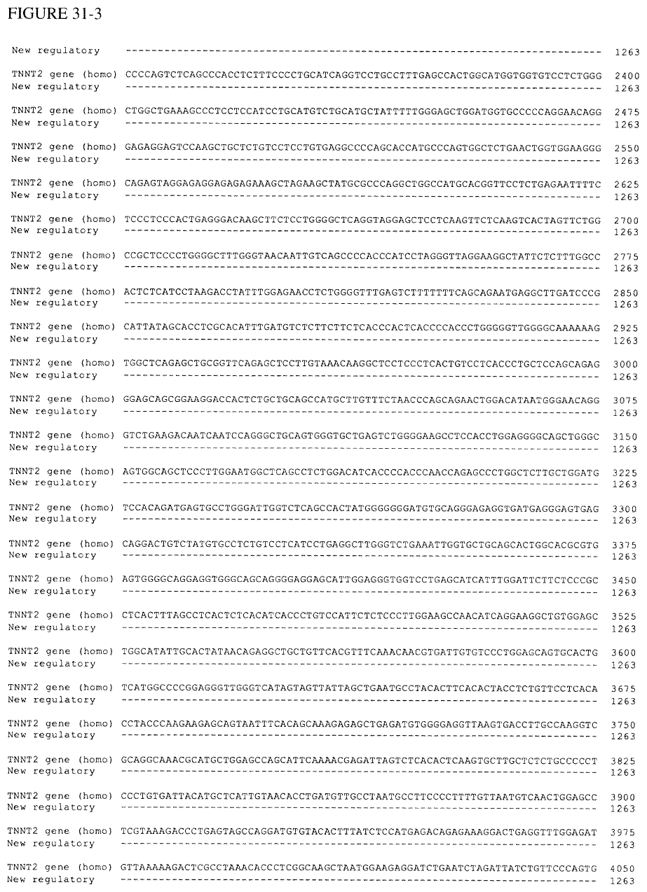

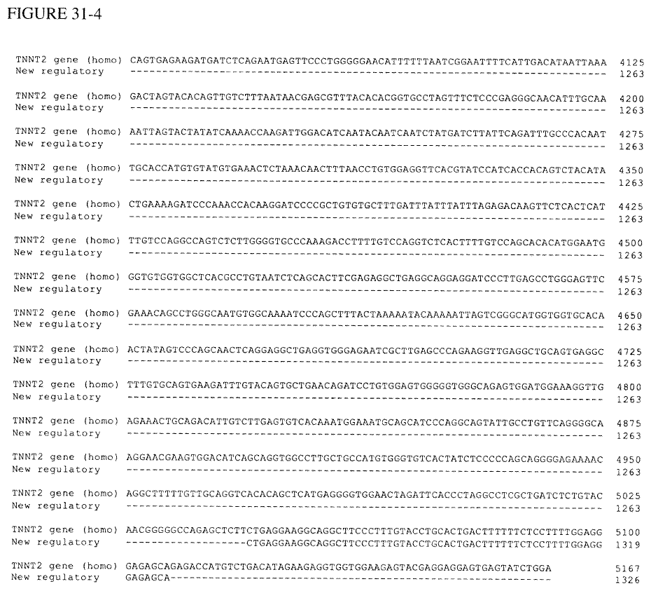

The TNNT2 gene sequence (5167 bp excerpted) shown in FIG. 31 is NCBI chromosome reference NC_000001.11 201378367..201373201 (gene is in antisense direction). Exon 1 is at bp 688-745 (NC_000001.11 201377680..201377623) and Exon 2 is at bp 5100-5154 (NC_000001.11 201373268..201373214) of TNNT2 gene. The hTNNT2 gene intronic region from bp 1662 to 2172 of FIG. 31 (NC_000001.11 201,376,706..201,376,196, bp 753-1263 of novel construct) contains an enhancer reported to enhance expression in vitro in a differentiated murine skeletal muscle cell line (C2C12) (Kwon et al. 2011). Sequence from the extreme 5′ and 3′ ends of Intron 1 are included to provide mRNA splicing and enhance transgene expression.

### Compositions 4-6

In the second proposed configuration, a cardiomyocyte-specific transcriptional cis-regulatory motif from human calsequestrin gene's first intron region NCBI chromosome reference NC_000001.11-115,768,786..115,768,977 previously described in (Chamberlain et al. 2018) and a −936-+42 bp human TNNT2 promoter fragment (NCBI chromosome reference NC_000001.11-201,378,613..201,377,636) are introduced to promote cardiac specific expression of the therapeutic transgenes.

Human cardiac troponin T sequences with the following gene coordinates are listed in U.S. Pat. No. 11,129,908: −569-+31 (TNNT2p-600 in Table 1 of U.S. Pat. No. 11,129,908); −469-+31 (TNNT2p-500 in Table 1 of U.S. Pat. No. 11,129,908); −369-+31 (TNNT2p-400 in Table 1 of U.S. Pat. No. 11,129,908); −269-+31 (TNNT2p-300 in Table 1 of U.S. Pat. No. 11,129,908). U.S. Pat. No. 11,312,943 discloses a synthetic human troponin T promoter sequence that features a unique 242 nucleotide sequence in addition to the human gene sequence having coordinates from −499 to +6. Werfel et al. discloses the use of a hTNNT2 promoter comprising −499++45 (designated −502-+42 om Werfel, et al.) and smaller fragments in AAV (Werfel et al. 2014).

Compositions 1-3 are the first description of the use of the skeletal muscle enhancer in the human TNNT2 gene as part of a regulatory sequence directing cardiac expression, either by itself or in conjunction with the human TNNT2 promoter. The promoter fragment in these compositions is larger than others previously described for expression vectors.

Other elements featuring the composition of rAAV's expression cassette are the SV40 16S Synthetic intron and poly adenylation elements which represent common feature in plasmid and viral expression vectors and are incorporated into the expression cassettes in order to promote high-level, efficient gene expression. Lastly, “Right” AAV2 Inverted Terminal Repeat (ITR) 3′ to the transgene is presented modified by a partial deletion of the terminal resolution site which allow hairpin formation of genome and originated a self-complementary (sc) vector that will result in maximized vector potency, while allowing for lower systemic doses. “Left” AAV2 ITR 5′ to the transgene is instead unmodified as it is required in cis for both viral DNA replication and packaging of the rAAV vector genome.

### Example 2

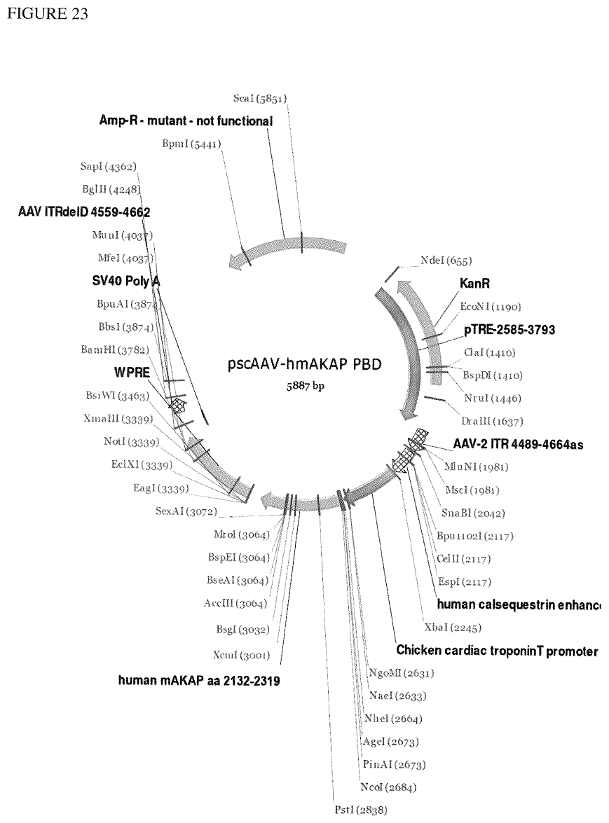

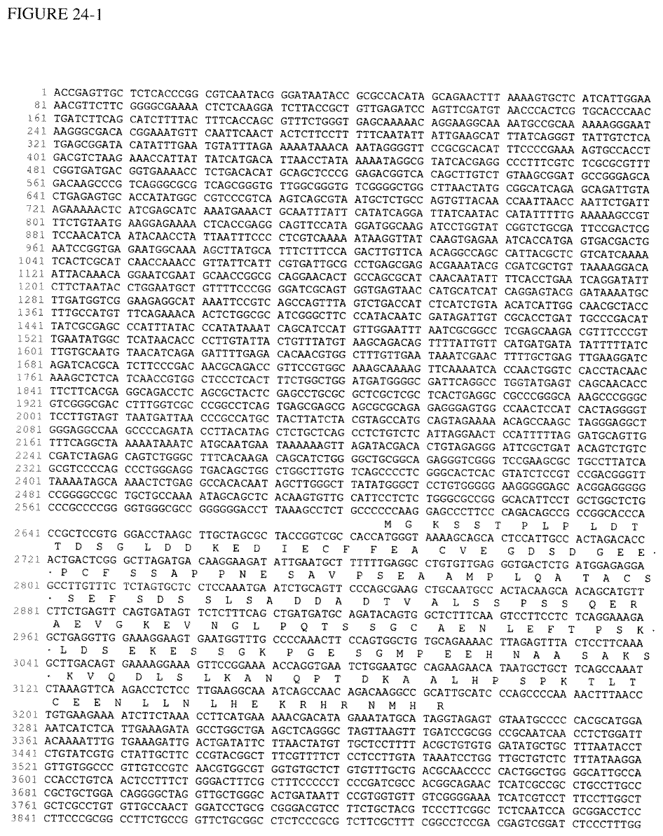

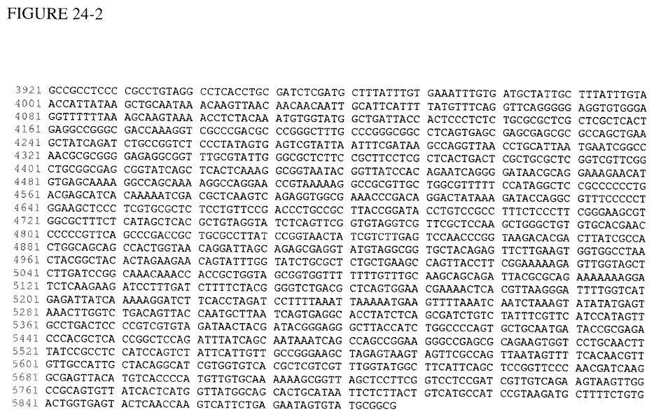

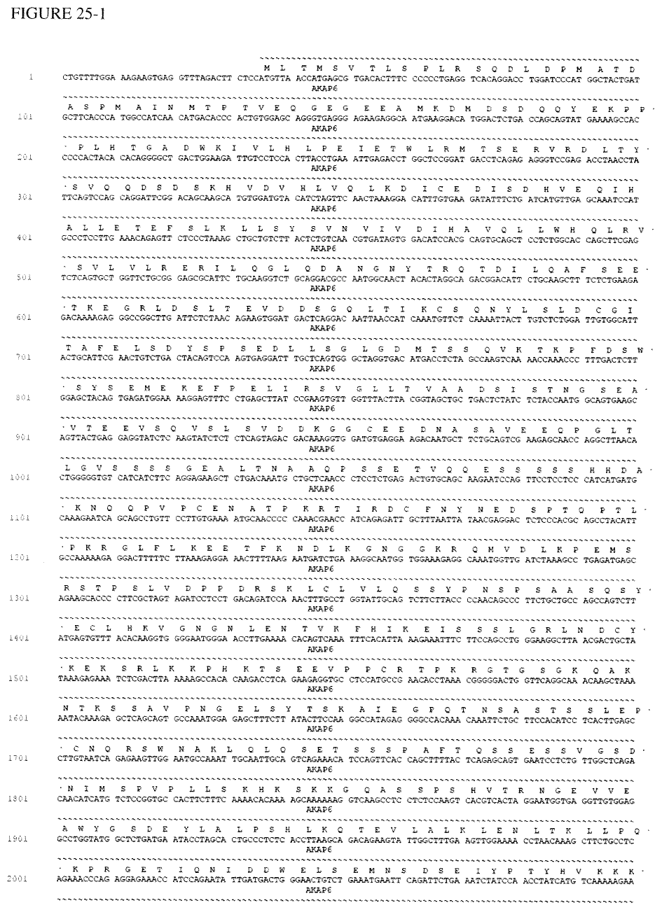

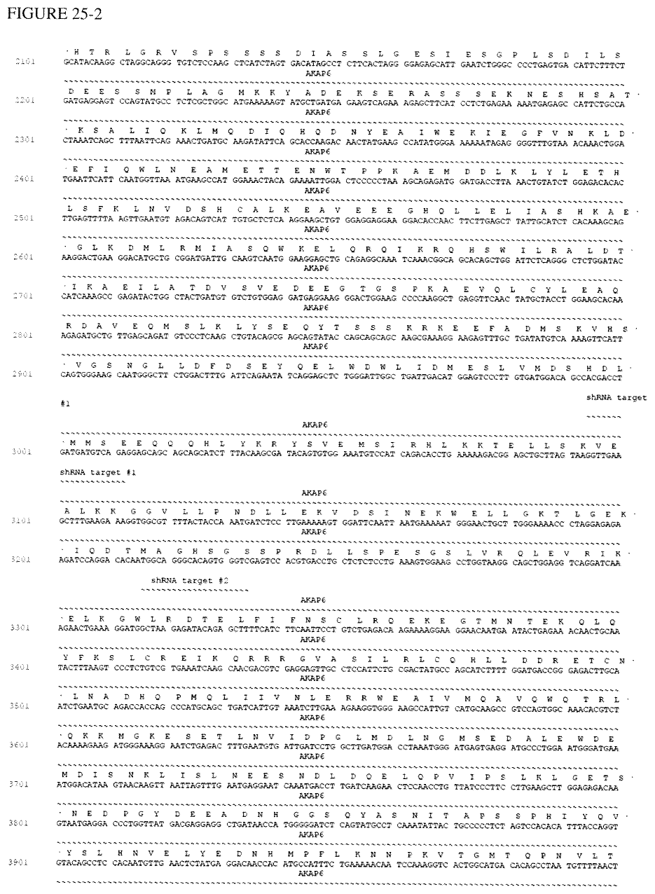

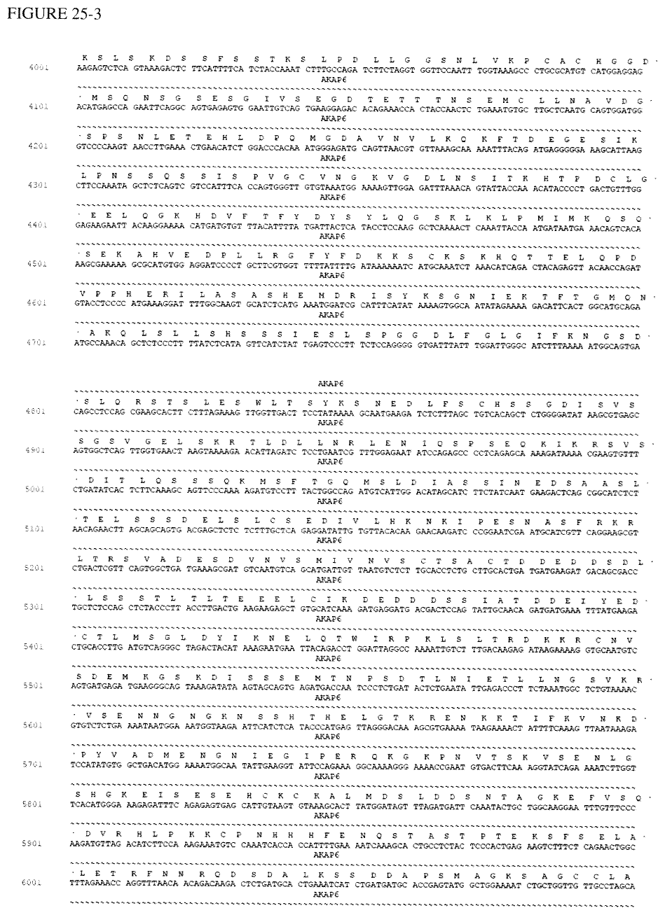

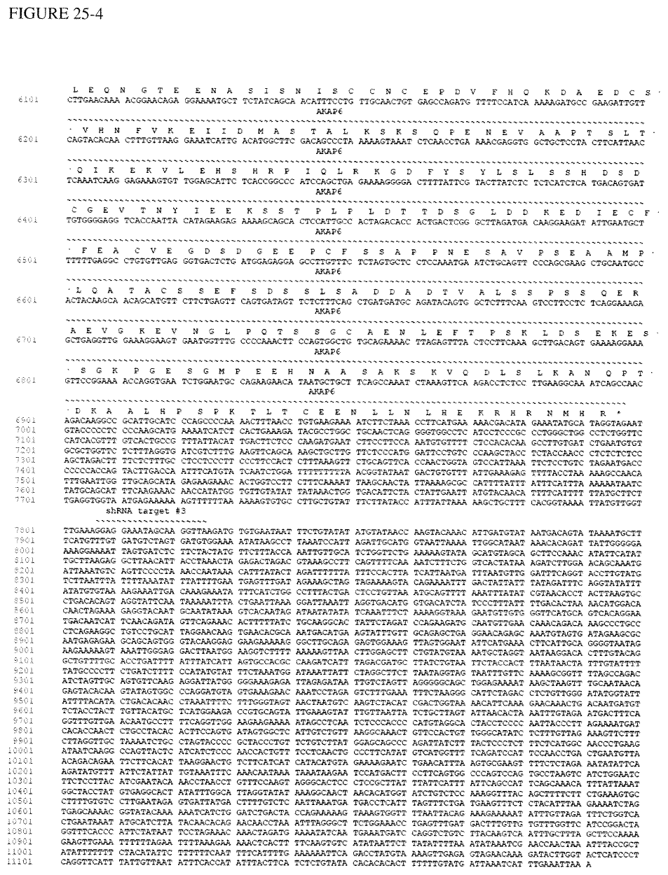

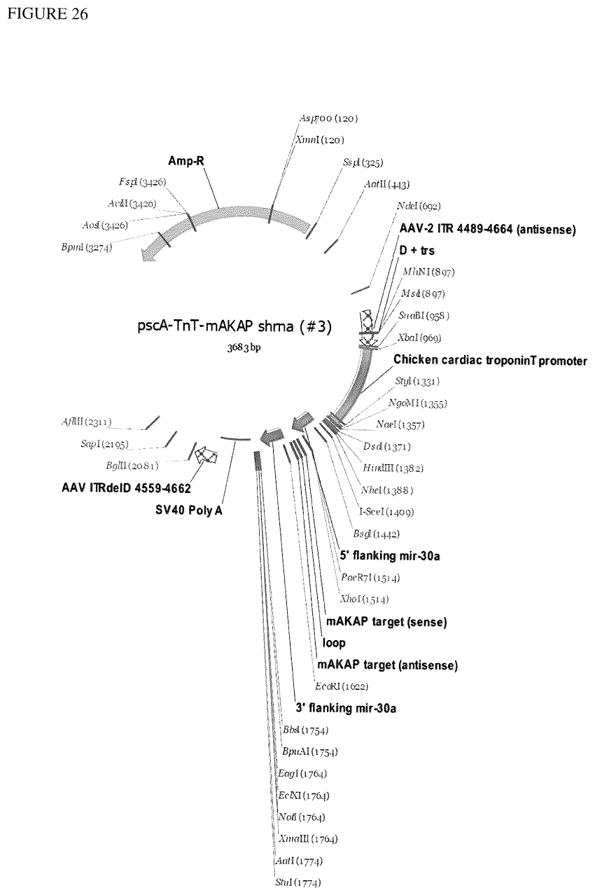

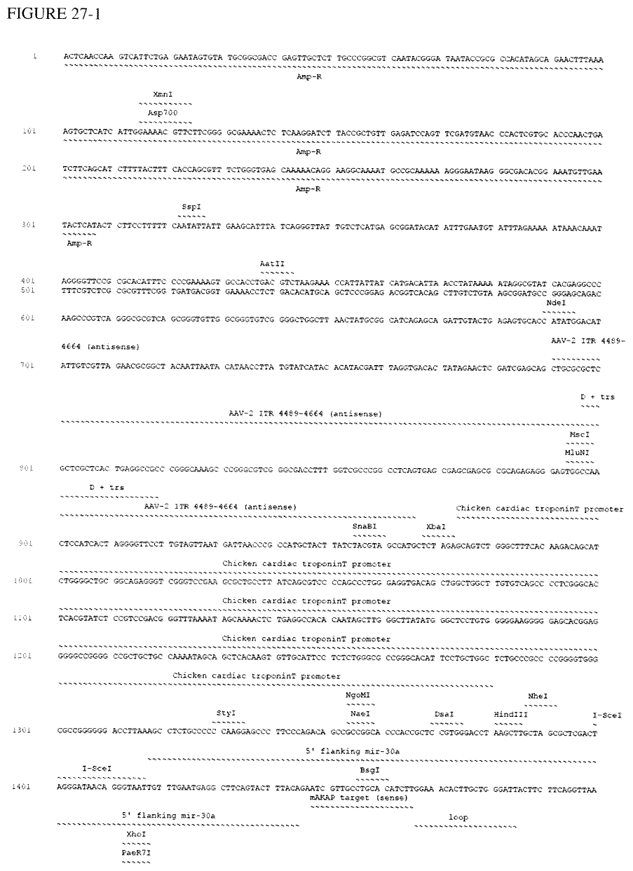

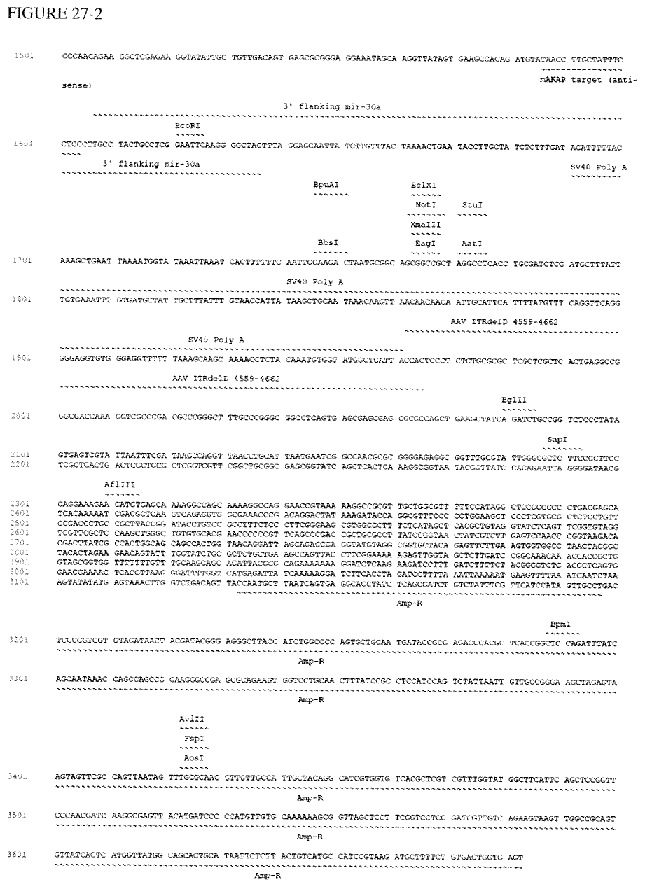

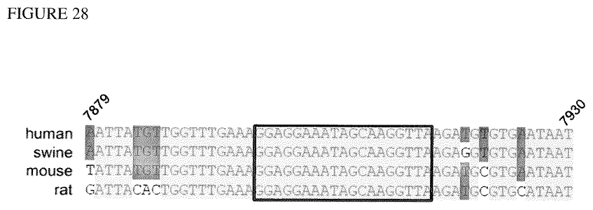

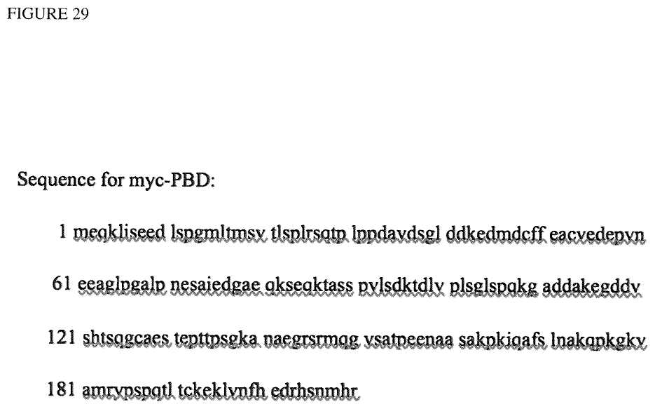

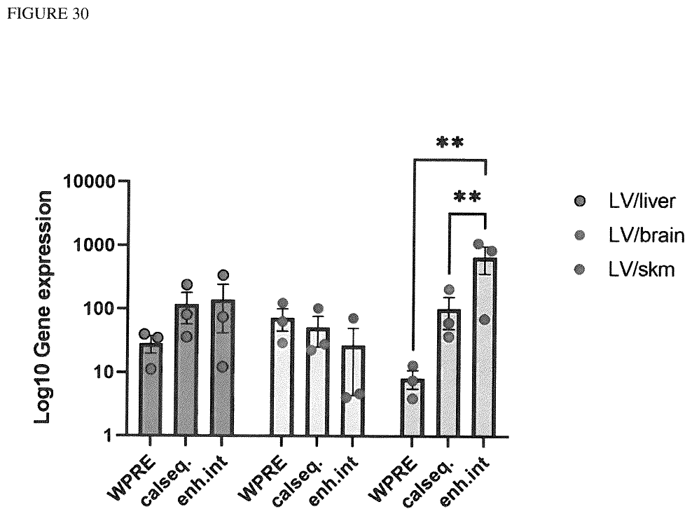

After production of AAV serotype 9 virus using compositions 1-6, expression directed by the two new layouts (layouts 1 and 2 abbreviated as “enh.int” and “calseq.”, respectively) was tested in 5-8 kg piglets by intravenous (iv) administration of ˜2E12 vg/kg of each AAV. Transgene expression and AAV biodistribution was evaluated post-mortem in liver, brain and cardiac tissues by quantitative polymerase chain reaction (qPCR] three months after AAV infusion. Unexpectedly, transgene expression (analyzed by reverse transcriptase—qPCR [qRT-PCR] and normalized to 18S RNA control) for the enh.int style compositions (shown in FIGS. 1-6) was ˜800-fold higher in the cardiac left ventricle than in skeletal muscle, while ˜3- and ˜20-fold higher in left ventricle than in liver and brain, respectively. AAV9 delivered intravenously in swine is preferentially delivered to the liver, with similar delivery to heart, skeletal muscle, and cerebral cortex (Li et al. 2022). Data presented in FIG. 30 show that the new enh.int composition significantly (p<0.05) enhanced cardiac left ventricular selectivity of transgene expression (transgene expression normalized by delivery of AAV9 genomes to the different tissues) over skeletal muscle ˜60 fold, when compared to “WPRE” AAV compositions similar to that in FIG. 23, while maintaining selectivity over liver and brain. In addition, expression in the left ventricle (normalized for delivery) was ˜4-fold higher for the new composition than the original “WPRE” composition.

### Example 3

### Ca²⁺/Calmodulin-Dependent Protein Kinase II (CaMKII)—a Key Regulator of the Myocyte

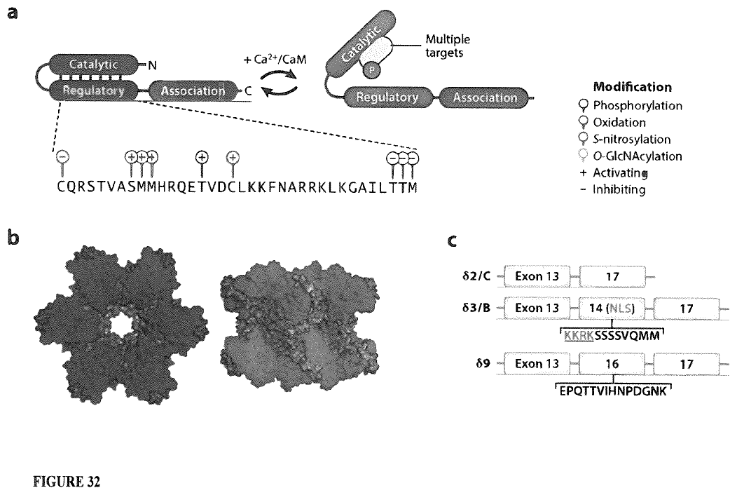

In response to G-protein coupled receptor (GPCR) signaling, CaMKII serves important roles in the regulation of cardiomyocyte ECC, gene transcription, inflammation, metabolism and cell survival (Hegyi, Bers et al. 2019, Reyes Gaido, Nkashama et al. 2023). CaMKII is expressed by 4 alternatively-spliced genes, of which CaMKII δ (CAMK2D) and 7 (CAMK2G) are highly expressed in the myocardium (Duran, Nickel et al. 2021). CaMKII family members share a common structure and post-translational modifications that can confer Ca²⁺-independence and prolong activation (FIG. 32). Functional differences among CaMKII isoforms have been observed that are likely due to differential localization, given their similar intrinsic catalytic activity (Zhang, Kohlhaas et al. 2007). For example, CaMKIIδB contains a nuclear localization signal and is cardioprotective, while CaMKII δC and δ9 tend to be more cytosolic and are associated with adverse remodeling (Duran, Nickel et al. 2021). CaMKII is present at the plasmalemma, transverse tubules, sarcoplasmic reticulum (SR), mitochondrion, nuclear envelope, and within the nucleus. With the exception of CaMKII targeting by A-kinase anchoring protein 18δ (AKAP18δ) to SR ryanodine receptor 2 (RyR2), SR Ca²⁺-ATPase type 2a (SERCA2a), and phospholamban (Pln) (Carlson, Aronsen et al. 2022), how CaMKII is localized within the myocyte remains poorly understood.

Elevated CaMKIIδ/γ expression and activity are associated with cardiovascular disease and are considered drivers of arrhythmia and pathological remodeling (Beckendorf, van den Hoogenhof et al. 2018, Duran, Nickel et al. 2021, Reyes Gaido, Nkashama et al. 2023). While CaMKII (γ, δC, but apparently not SB) physiologically regulates diverse ion channels (Zhang, Kohlhaas et al. 2007, Kreusser, Lehmann et al. 2014), fine-tuning excitation-contraction coupling (Hegyi, Bers et al. 2019), chronically elevated CaMKII activity can induce excess SR Ca²⁺ leak and arrhythmia (Maier and Bers 2007, Beckendorf, van den Hoogenhof et al. 2018). Transgenic expression of the major cardiac CaMKIIδ variants induces hypertrophy in vivo and, with the exception of SB, rapidly promotes heart failure (Duran, Nickel et al. 2021). Conversely, while not inhibiting the initial induction of hypertrophy by pressure overload, CaMKIIδ deletion inhibits the eccentric hypertrophy and heart failure associated with long-term pressure overload (Ling, Zhang et al. 2009). Post-myocardial infarction, CaMKIIδ/γ gene deletion protects against pathological remodeling (Weinreuter, Kreusser et al. 2014). Reactive oxygen species (ROS)-activated CaMKII δC and δ9 worsen myocyte death through mitochondrial and NF-κB death and inflammatory pathways, as well as impaired DNA repair (Feng and Anderson 2017, Yao, Li et al. 2022). Driven by ROS and hyperglycemia, CaMKII activation by oxidation and O-linked-N-acetylglucosaminylation (0-GlcNAcylation) is relevant to diabetic cardiomyopathy (Hegyi, Bers et al. 2019, Veitch, Power et al. 2021).

The extensive data showing that CaMKII signaling promotes heart failure and arrythmia has compelled efforts to develop CaMKII-directed therapies, with it generally argued that loss of cardiomyocyte CaMKII activity would be well-tolerated and beneficial (Nassal, Gratz et al. 2020, Lebek, Chemello et al. 2023). There are diverse physiologic functions of CaMKII family members, including the prominent role of brain CaMKII α and β in learning and memory (Beckendorf, van den Hoogenhof et al. 2018). In addition, CaMKII is required for physiological sympathetic responses, particularly at the sino-atrial node (Wu, Gao et al. 2009). Moreover, through the regulation of Pln and RyR2, preserved CaMKII activity is essential during early pressure overload disease, as well as in the contractile adaptation to exercise (Burgos, Yeves et al. 2017, Baier, Klatt et al. 2020). Further, nuclear CaMKIIδB is cardioprotective through cAMP Response Element-Binding Protein (CREB) Serine 133 phosphorylation (Wang, Xu et al. 2022).

mAKAPβ, CaMKII, and HDAC4

In cardiomyocytes, the 230-kDa scaffold protein muscle A-kinase anchoring protein R (mAKAPβ/AKAP6β) organizes signalosomes that integrate Ca²⁺, cAMP, phosphatidylinositol 4-phosphate, mitogen-activated protein kinase, and hypoxic upstream signaling regulating myocyte transcription factors and class IIa histone deacetylases (HDACs) (Dodge-Kafka, Gildart et al. 2019). Through the post-translational modification of these gene regulators, mAKAPβ signalosomes influence both the extent of remodeling and the quality of cardiac hypertrophy in terms of concentric vs. eccentric morphology (Li, Tan et al. 2020). mAKAPβ is required for the induction of pathological cardiac remodeling and heart failure by chronic pressure overload, catecholamine infusion, and MI (Kritzer, Li et al. 2014, Martinez, Li et al. 2023). mAKAPβ is not required for cardiac development, and mAKAPβ cardiac-specific knock-out has no apparent adverse effect on the response to swim training (Kritzer, Li et al. 2014). Recently, it was discovered that rat mAKAPβ 1694-1833 that binds RSK3 also binds CaMKIIδ and CaMKIIγ.

Both pacing and pressure overload induce accumulation of active Threonine-287 autophosphorylated CaMKII at the myocyte nuclear envelope (Ljubojevic-Holzer, Herren et al. 2020). CaMKII phosphorylates HDAC4, inducing 14-3-3 binding and nuclear export and de-repressing myocyte enhancer factor 2 (MEF2)-dependent gene expression that drives pathological remodeling (Backs, Song et al. 2006, Zhang, Kohlhaas et al. 2007, Li, Cai et al. 2011). Although CaMKII phosphorylates sites conserved among class IIa HDACs, CaMKII preferentially phosphorylates HDAC4 Ser-467/632 due to a CaMKII docking site exclusively on HDAC4 (aa 585-608) (Backs, Song et al. 2006, Backs, Backs et al. 2009). It was previously reported that during long term pressure overload, increased HDAC4 phosphorylation was mAKAPβ-dependent (Kritzer, Li et al. 2014), consistent with CaMKII binding to mAKAPβ.

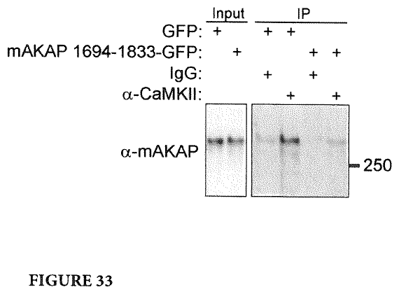

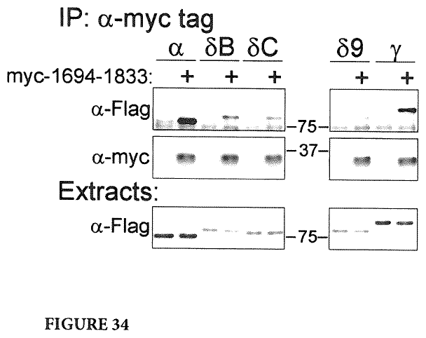

To identify novel RBD binding partners, myc- and green-fluorescent protein (GFP) tagged mAKAP 1694-1833 was expressed in adult rat ventricular myocytes by adenoviral infection, and, following dithiobis(succinimidyl propionate (DSP) crosslinking, protein complexes were immunoprecipitated under stringent conditions with myc tag antibodies and analyzed by mass spectroscopy. Along with other candidate interactors, CaMKII 7 and 6 were highly enriched in mAKAP immunoprecipitates (with 11 and 16 peptides identified, respectively, by mass spectroscopy). The binding of CaMKII to mAKAPβ was validated by co-immunoprecipitation of endogenous proteins from neonatal rat ventricular myocytes infected with adenovirus expressing either myc-GFP or myc-GFP-mAKAP 1694-1833 (FIG. 33). mAKAPβ immunoprecipitation with CaMKII antibodies was competed by expression of the 1694-1833 fusion peptide, validating kinase binding to both the scaffold and competing peptide. When co-expressed in COS-7 cells, mAKAP 1694-1833 bound diverse CaMKII isoforms (FIG. 34), suggesting that mAKAPβ binds a CaMKII domain common to all CaMKII family members.

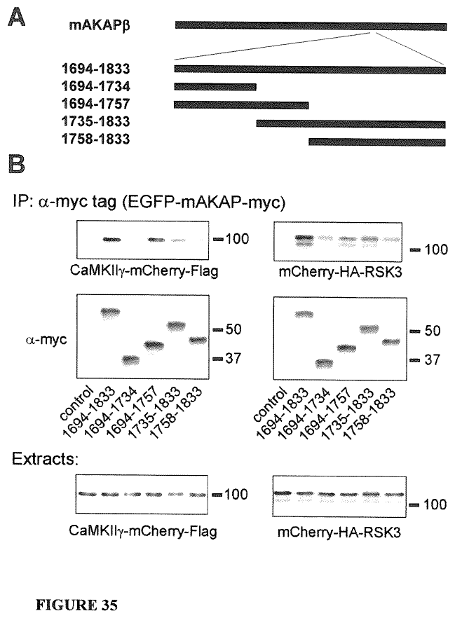

Preliminary mapping studies have been performed to discern whether the binding sites for RSK3 and CaMKII are distinct within mAKAP aa 1694-1833 (FIG. 35). Although the relative binding of RSK3 and CaMIKIIγ to mAKAP aa 1694-1757 and 1735-1833 is different, both kinases apparently bind a large, overlapping region of aa 1694-1833.

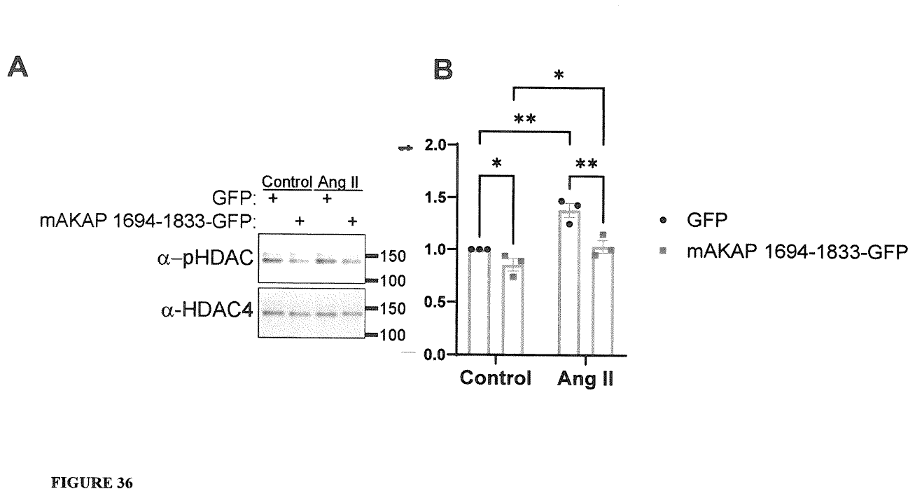

HDAC4 phosphorylation on CaMKII sites during chronic pressure overload is dependent upon mAKAPβ expression (Kritzer, Li et al. 2014). The present data show that both basal and Angiotensin II-induced HDAC4 phosphorylation in neonatal myocytes is inhibited by mAKAP 1694-1833 expression, consistent with CaMKII inhibition (FIG. 36).

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

### REFERENCES

Ahn et al., 2007. Proc Natl Acad Sci USA, 104: 2979-84.
Akira et al., 2006. Cell, 124: 783-801.
Anjum et al., 2008. Nat Rev Mol Cell Biol, 9: 747-58.
Backs et al. 2009. Proc Natl Acad Sci USA, 106(7): 2342-2347.
Backs et al. 2006. J Clin Invest, 116(7): 1853-1864.
Backs et al. 2011. J Cell Biol, 195: 403-15.
Baier et al. 2020. J Mol Cell Cardiol 138: 212-221.
Beckendorf et al. 2018. Basic Res Cardiol, 113(4): 29.
Becker et al. 2021. Elife, 10.
Bers et al. 2006. Biochem J, 396: e1-3.
Bione et al. 1994. Nat Genet, 8: 323-7.
Black and Olson. 1998. Annu Rev Cell Dev Biol, 14: 167-96.
Bode et al. 2011. Expert Rev Vaccines, 10: 499-511.
Bonne et al. 1999. Nat Genet, 21: 285-8.
Bourajjaj et al. 2008. J Biol Chem, 283: 22295-303.
Bueno et al. 2004. Circ Res, 94: 91-9.
Bueno et al. 2002. Proc Natl Acad Sci USA, 99: 4586-91.
Burchfield et al. 2013. Circulation, 128: 388-400.
Burgos et al. 2017. J Mol Cell Cardiol, 112: 16-26.
Carlisle et al. 2004. Biochem J, 381: 587-92.
Carlson et al. 2022. Circ Res, 130(1):27-44.
Chamberlain et al. 2018. Hum Gene Ther, 29: 927-37.
Dobrev and Wehrens. 2014. Circ Res, 114: 1311-9; discussion 19.
Dodge-Kafka et al. 2010. J Biol Chem, 285: 11078-86.
Dodge-Kafka et al. 2018. J Mol Cell Cardiol, 118: 13-25.
Dodge-Kafka et al. 2019. Cell Signal, 63: 109357.
Dodge-Kafka and Kapiloff. 2006. Eur J Cell Biol, 85: 593-602.
Dodge-Kafka et al. 2005. Nature, 437: 574-8.
Dodge et al. 2001. EMBO J, 20: 1921-30.
Dulhunty et al. 2007 Pharmacol Ther, 113: 247-63.
Duran et al. 2021. Front Cell Dev Biol, 9: 644630.
Elbashir et al. 2001. Nature, 411: 494-8.
Escobar et al. 2011. J Mol Cell Cardiol, 50: 451-9.
Fatkin et al. 1999. N Engl J Med, 341: 1715-24.
Faul et al. 2007. Mol Cell Biol, 27: 8215-27.
Feng et al. 2017. J Mol Cell Cardiol, 103: 102-109.
Fish et al. 2013. Circulation Heart failure 6: 310-317.
Friday et al. 2003. Differentiation, 71: 217-27.
Gerber et al. 2016. Circ Heart Fail, 9: e002460.
Go et al. 2014. Committee American Heart Association Statistics, and Subcommittee Stroke Statistics. Circulation, 129: e28-e292.
Grossman et al. 1975. Clin Invest, 56: 56-64.
Guo et al. 2015. Int J Mol Med 5: 1159-68.
Hakem Zadeh et al. 2019. Physiol Rep, 7: e14144.
Hamilton et al. 2021. Front Immunol, 12: 675897.
Hartmann et al. 1999. Proc Natl Acad Sci USA, 96: 9305-10.
Hawash et al. 2021. Proc Natl Acad Sci U SA, 118(13): e2015855118.
Heidenreich et al. 2013. Circ Heart Fail, 6: 606-19.
Heidenreich et al. 2022. Circulation, 145: e895-e1032.
Heineke and Molkentin. 2006. Nat Rev Mol Cell Biol, 7: 589-600.
Hegyi et al. 2019. J Mol Cell Cardiol, 127: 246-25.
Hill and Olson. 2008. NEngl J Med, 358: 1370-80.
Hoffmann et al. 1999. EMBO J, 18: 893-903.
Holt et a. 2019. Sci Rep, 9: 14202.
Houser. 2014. Circ Res, 114: 1320-7; discussion 27.
Ishikawa et al. 2018. Circ Res 123: 601-613.
Jugdutt 2003. Curr Drug Targets Cardiovasc Haematol Disord, 3: 1-30.
KannegantiT. et al. 2007. Immunity, 27: 549-59.
Kapiloff et al. 2001. J Cell Sci, 114: 3167-76.
Kapiloff et al. 2009. J Biol Chem, 284: 23540-6.
Kapiloff et al. 1999. J Cell Sci, 112 (Pt 16): 2725-36.
Kato et al. 2000. J Biol Chem, 275: 18534-40.
Kehat et al. 2011. Circ Res, 108: 176-83.
Kieserman et al. 2019. JAm Heart Assoc 8: e012239.
Kim et al. 2008. J Clin Invest, 118: 124-32.
Kimura et al. 2010. Circ Res, 106: 961-70.
Kreusser et al. 2014. Circulation, 130(15):1262-1273.
Kritzer et al. 2012. J Mol Cell Cardiol, 52: 351-8.
Kritzer et al. 2014. Circ Heart Fail, 7: 663-72.
Kuzmin et al. 2021. Nat Rev Drug Discov 20: 173-174.
Kwon et al. 2011. PLoS Comput Biol, 7: e1002256.
Lebek et al. 2023. Science, 379(6628): 179-185.
Li et al. 2019. J Biol Chem, 294: 2543-54.
Li et al. 2011. Biochem Biophys Res Commun, 409(1): 125-130.
Li et al. 2022. Physiol. Genomics 54(7):261-272.
Li et al. 2013. Circ Res, 112: 128-39.
Li et al. 2010. J Mol Cell Cardiol, 48: 387-94.
Li et al. 2020. Circulation, 142: 2138-54.
Li et al. 2013. Exp Cell Res, 319: 447-54.
Ling et al. 2009. J Clin Invest, 119(5):1230-1240.
Liu et al. 2021. Science translational medicine 13: 2021.
Ljubojevic-Holzer et al. 2020. Circ Res, 127(9): 1159-1178.
Mackenzie et al. 2008. Biochem J, 411: 361-9.
Maier et al. Cardiovasc, 2007. Res 73(4):631-640.
Martinez et al. 2023. Gene Ther, 30(7-8): 543-551.
Marx et al. 2000. Cell, 101: 365-76.
Maxwell et al. 1999. Nature, 399: 271-5.
McCarty et al. 2001. Gene Ther. 8(16):1248-54.
McCartney et al. 1995. Journal of Biological Chemistry, 270: 9327-33.
Michel et al. 2005. Mol Cell, 20: 661-72.
Monovich et al. 2010. FEBS Lett, 584: 631-7.
Moretti et al. 2020. Nat Med 26: 207-214.
Muchir et al. 2000. Hum Mol Genet, 9: 1453-9.
Nakamura and Sadoshima. 2018. Nat Rev Cardiol, 15: 387-407.
Nassal et al. 2020. Front Pharmacol, 11: 35.
Naya and Olson. 1999. 'Curr Opin Cell Biol, 11: 683-8.
Naya et al. 1999. Development, 126: 2045-52.
Negro et al. 2008. Prog Pediatr Cardiol, 25: 51-56.
Newlon et al. 1999. Nat Struct Biol, 6: 222-7.
Nicol et al. 2001. EMBO J, 20: 2757-67.
Ohh et al. 2000. Nat Cell Biol, 2: 423-7.
Ohto et al. 2018. Immunity, 48: 649-58 e4.
Ohto et al. 2015. Nature, 520: 702-5.
Pare et al. 2005. J Cell Sci, 118: 5637-46.
Pare et al. 2005. Exp Cell Res, 303: 388-99.
Passariello et al. 2013. Am J Physiol Heart Circ Physiol, 305: H1010-9.
Passariello et al. 2016. J Mol Cell Cardiol, 93: 98-105.
Pleger et al. 2011. Science translational medicine 3: 92ra64.
Pohar et al. 2017. J Immunol, 198: 2093-104.
Potthoff and Olson. 2007. Development, 134: 4131-40.
Prasad et al. 2011. Gene Ther, 18: 43-52.
Rababa'h et al. 2013. J Mol Biol, 425: 3277-88.
Ranganathan et al. 2006. Arch Biochem Biophys, 449: 8-16.
Reyes Gaido et al. 2023. Annu Rev Pharmacol Toxicol, 63: 249-272.
Sadoshima et al. 1995. Circ Res, 76: 1-15.
Schiattarella and Hill. 2015. Circulation, 131: 1435-47.
Schulze et al. 2003. J Biol Chem, 278: 28849-55.
Scott et al. 2013. Annu Rev Pharmacol Toxicol, 53: 187-210.
Scott and Pawson. 2009. Science, 326: 1220-4.
Sette and Conti. 1996 J Biol Chem, 271: 16526-34.
Sharma and Kass. 2014. Circ Res, 115: 79-96.
Shirley et al. 2020. Mol Ther, 28: 758-70.
Tahtinen et al. 2022. Nat Immunol, 23: 532-42.
Takeishi et al. 2002. Cardiovasc Res, 53: 131-7.
ThermoFisher Scientific, retrieved Jun. 16, 2017 <https://http://www.thermofisher.com/us/en/home/references/ambion-tech-support/rnai-sirna/general-articles/-sirna-design-guidelines.html>.
Tsao et al. 2023. ‘Heart Disease and Stroke Statistics-2023 Update: A Report From the American Heart Association’, Circulation.
Turcotte, M et al. 2022. J Mol Cell Cardiol, 172: 26-40.
Valdivia et al. 1995. Science, 267: 1997-2000.
Vargas et al. 2012. Cell Signal, 24: 1496-503.
Veitch et al. 2021. Front Pharmacol, 12: 695401.
Vergarajauregui et al. 2020. Elife, 9.
Wang et al. 2019. Nat Rev Drug Discov 18: 358-378, 2019.
Wang et al. 2005. J Biol Chem, 280: 16705-13.
Wang et al. 2015. EBioMedicine, 2: 1880-7.
Wang et al. 2022. Circulation, 145(14): 1067-1083.
Weinreuter et al. EMBO Mol Med, 6(10): 1231-1245.
Werfel et al. 2014. Cardiovasc Res, 104: 15-23.
Wilkins et al. 2004. Circ Res, 94: 110-8.
Wilkins et al. 2002. Mol Cell Biol, 22: 7603-13.
Wong et al. 2008. Sci Signal, 1: ra18.
Wright 2020. Mol Ther, 28: 1756-58.
Wu et al. 2009. Proc Natl Acad Sci USA, 106(14): 5972-5977.
Wu et al. 2001. EMBO J, 20: 6414-23.
Xiang et al. 2020. Mol Ther, 28: 771-83.
Xie et al. 2013. Circulation, 128: 1021-30.
Xie and Hill. 2013. Trends Cardiovasc Med, 23: 229-35.
Yang et al. 1998. J Cell Biol, 142: 511-22.
Yao et al. 2022. Circ Res, 130(6): 887-903.
Zakhary et al. 2000. J Biol Chem, 275: 41389-95.
Zhang et al. 2007. J. Biol Chem, 282(48): 35078-35087.
Zhang et al. 2011. J Biol Chem, 286: 23012-21.
Zhang et al. 2013. Cell, 153: 216-27.
Zhang et al. 2007. Hum Mol Genet, 16: 2816-33.
Zhao et al. 1995. Mol Cell Biol, 15: 4353-63.
Zhu et al. 2009. J Clin Invest, 119: 2388-98.

What is claimed is:

1. A composition comprising a regulatory nucleotide sequence for expression of a second nucleotide sequence in a cardiac myocyte, wherein said regulatory nucleotide sequence comprises an intronic sequence comprising a splicing consensus site, wherein said intronic sequence is from the human cardiac troponin T gene. (hTNNT).

2. The composition of claim 1, further comprising a TNNT2 promoter sequence.

3. The composition of claim 1, wherein the regulatory nucleotide sequence is in a vector.

4. The composition of claim 3, further comprising a transgene.

5. The composition of claim 4, wherein the transgene is a muscle A-kinase anchoring protein R (mAKAPβ) sequence.

6. The composition of claim 5, wherein the mAKAPβ sequence is an shRNA.

7. The composition of claim 6, wherein the shRNA comprises GGTTGAAGCTTTGAAGAAA (SEQ ID NO: 77), GCTAAGAGATACAGAGCTT (SEQ ID NO: 78) or GGAGGAAATAGCAAGGTTA (SEQ ID NO: 79).

8. The composition of claim 3, wherein the vector encodes an amino acid sequence having at least 80% sequence homology to a fragment of mAKAPβ.

9. The composition of claim 8, wherein the vector encodes an amino acid sequence having at least 90% sequence identity to a fragment of mAKAPβ.

10. The composition of claim 9, wherein the amino acid sequence encodes a fragment of mAKAPβ.

11. The composition of claim 8, wherein the amino acid sequence binds a kinase.

12. The composition of claim 11, wherein the kinase is p90 ribosomal S6 kinase 3 (RSK3).

13. The composition of claim 12, wherein amino acid sequence inhibits the binding of mAKAPβ to RSK3.

14. The composition of claim 10, wherein the amino acid sequence has at least 80% sequence homology to amino acids 1694-1757, 1735-1833 or 1694-1833 of mAKAβ.

15. The composition of claim 14, wherein the amino acid sequence has at least 90% sequence identity to amino acids 1735-1833 of mAKAβ.

16. The composition of claim 12, wherein the amino acid sequence comprises a RSK3 binding domain (RBD) of mAKAPβ.

17. The composition of claim 15, wherein the RBD comprises amino acids 1735-1833 of SEQ ID NO:12.

18. The composition of claim 11, wherein the amino acid sequence binds protein phosphatase 2A (PP2A).

19. The composition of claim 18, wherein amino acid sequence inhibits the anchoring PP2A to mAKAPβ.

20. The composition of claim 19, wherein the amino acid sequence has at least 80% sequence homology to amino acids 2132-2319 of mAKAP.

22. The composition of claim 20, wherein the amino acid sequence has at least 90% sequence identity to amino acids 2132-2319 of mAKAP.

23. The composition of claim 20, wherein the amino acid sequence comprises a PP2A binding domain (PBD) of mAKAPβ.

24. The composition of claim 23, wherein the PBD comprises amino acids 2132-2319 of SEQ ID NO:12.

25. The composition of claim 11, wherein the kinase is Ca²⁺/calmodulin-dependent protein kinase II (CaMKII).

26. The composition of claim 3, wherein the vector is adeno-associated virus (AAV).

27. The composition of claim 3, wherein the vector further comprises SV40 polyadenylation sequences.

28. The composition of claim 5, wherein human mAKAP amino acids 2132-2319 (SEQ ID NO:12) has been modified at one or more of the following positions: TCG at amino acid 2144 has been modified to TCA; AGC at amino acid 2183 has been modified to AGT; TCC at amino acid 2256 has been modified to TCA; GCC at amino acid 2291 has been modified to GCA; or CGA at amino acid 2313 has been modified to AGA.

29. The composition of claim 5, wherein human mAKAP amino acids 1696-1835 (SEQ ID NO:12) encoding RBD has been modified at one or more of the following positions: CCG at amino acid 1712 has been modified to CCA; TCG at amino acid 1714 has been modified to TCT; TCG at amino acid 1717 has been modified to TCT; CGT at amino acid 1721 has been modified to AGA; CGT at amino acid 1724 has been modified to AGA; AGC at amino acid 1730 has been modified to AGT; AGC at amino acid 1753 has been modified to AGT; and GAC at amino acid 1775 has been modified to GAT.

30. A method of treating or preventing heart disease, comprising administering to cardiac cells of a patient the vector of claim 3.

31. A method of treating or preventing heart disease, comprising administering to cardiac cells of a patient the vector of claim 6, wherein the method inhibits the expression of mAKAP.


SEQUENCE LISTING (82 sequences)

SEQ ID NO: 1 - DNA, 5707 bp, synthetic construct
  source 1..5707: mol_type = other DNA
cattctgagaatagtgtatgcggcgaccgagttgctctcacccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttcaattcaactactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctactcgagcctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgtctagagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagctgctgttctgaggtaaggctcgggcagggctcttgcctggtgtgtaaggaatttcagagcacattcctggcctgctgtagccgggaagctacctgccctcccaaattccttttctgacccatgtcttagagggcgaatcttccttctctctgcaaaagtggaatctgagaggatcaggttgctggctgcctcctatttctgacccccacttccactcttcctgccctctccccatccccttacccccgctctcctggtgggctaacagctgttggcttaggaaaatcccattcgagtgctggggacaaggactgaggggtggggcctgtgtttctcagaaactcggagactgtttctacagggacagaactcaaggctctcctgctttgtagttggttaactgaagtcagagaggctaggtgacttaaataaggccacacagcaaaatctgggttttaagcccagattttttgaccccaaggtcagtatgccttatgccttctattgtctttgctttactccccaagaaaggtgaccctgtgaactgaggaaggcaggcttccctttgtacctgcactgacttttttctccttttggagggagagcagctagcaggactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgcgggaggaaatagcaaggttatagtgaagccacagatgtataaccttgctatttcctcccttgcctactgcctcggaattcaaggggctactttaggagcaattatcttgtttactaaaactgaataccttgctatctctttgatacatttttacaaagctgaattaaaatggtataaattaaatcacttttttcaattggaagactaatggcagcctagagctcaggcctcacctggatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattaccactccctctctgcgcgctcgctcgctcactgaggccgggcgaccaaaggtcgcccgacgcccgggctttgcccgggcggcctcagtgagcgagcgagcgcgccagctgaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaatcagccagccggaagggccgagcgcagaagtggtcctgcaactttatccgcctccatccagtctattcattgttgccgggaagctagagtaagtagttcgccagttaatagttttcacaacgttgttgccattgctacaggcatcgtggtgtcacgctcgtcgtttggtatggcttcattcagctccggttcccaacgatcaaggcgagttacatgtcaccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagt

SEQ ID NO: 2 - DNA, 5773 bp, synthetic construct
  source 1..5773: mol_type = other DNA
cattctgagaatagtgtatgcggcgaccgagttgctctcacccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttcaattcaactactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctactcgagcctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgtctagagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagctgctgttctgaggtaaggctcgggcagggctcttgcctggtgtgtaaggaatttcagagcacattcctggcctgctgtagccgggaagctacctgccctcccaaattccttttctgacccatgtcttagagggcgaatcttccttctctctgcaaaagtggaatctgagaggatcaggttgctggctgcctcctatttctgacccccacttccactcttcctgccctctccccatccccttacccccgctctcctggtgggctaacagctgttggcttaggaaaatcccattcgagtgctggggacaaggactgaggggtggggcctgtgtttctcagaaactcggagactgtttctacagggacagaactcaaggctctcctgctttgtagttggttaactgaagtcagagaggctaggtgacttaaataaggccacacagcaaaatctgggttttaagcccagattttttgaccccaaggtcagtatgccttatgccttctattgtctttgctttactccccaagaaaggtgaccctgtgaactgaggaaggcaggcttccctttgtacctgcactgacttttttctccttttggagggagagcagctagcagccaccatggtggagctctctctttgctcagaggatattgtgttacacaagaacaagatcccagaatctaatgcatctttcaggaagagactgactagatcagtggctgatgaaagtgatgtcaatgtcagcatgattgttaatgtctcttgcacctctgcttgcactgatgatgaagatgacagtgacctgctctccagctctacccttaccttgactgaagaagagctgtgcatcaaagatgaggatgatgactccagtattgcaacagatgatgaaatttatgaagactgcaccttgatgtcagggctagactacataaagaatgaattacagacctggattaggccaaaattgtctttgacaagagataagaaaaggtgcaatgtcagtgatgagatgaagggcagtaaagatataagtagcagtgagtaggagctcaggcctcacctggatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattaccactccctctctgcgcgctcgctcgctcactgaggccgggcgaccaaaggtcgcccgacgcccgggctttgcccgggcggcctcagtgagcgagcgagcgcgccagctgaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaatcagccagccggaagggccgagcgcagaagtggtcctgcaactttatccgcctccatccagtctattcattgttgccgggaagctagagtaagtagttcgccagttaatagttttcacaacgttgttgccattgctacaggcatcgtggtgtcacgctcgtcgtttggtatggcttcattcagctccggttcccaacgatcaaggcgagttacatgtcaccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagt

SEQ ID NO: 3 - DNA, 5917 bp, synthetic construct
  source 1..5917: mol_type = other DNA
cattctgagaatagtgtatgcggcgaccgagttgctctcacccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttcaattcaactactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctactcgagcctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgtctagagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagctgctgttctgaggtaaggctcgggcagggctcttgcctggtgtgtaaggaatttcagagcacattcctggcctgctgtagccgggaagctacctgccctcccaaattccttttctgacccatgtcttagagggcgaatcttccttctctctgcaaaagtggaatctgagaggatcaggttgctggctgcctcctatttctgacccccacttccactcttcctgccctctccccatccccttacccccgctctcctggtgggctaacagctgttggcttaggaaaatcccattcgagtgctggggacaaggactgaggggtggggcctgtgtttctcagaaactcggagactgtttctacagggacagaactcaaggctctcctgctttgtagttggttaactgaagtcagagaggctaggtgacttaaataaggccacacagcaaaatctgggttttaagcccagattttttgaccccaaggtcagtatgccttatgccttctattgtctttgctttactccccaagaaaggtgaccctgtgaactgaggaaggcaggcttccctttgtacctgcactgacttttttctccttttggagggagagcagctagcagccaccatgggtaaaagcagcactccattgccactagacaccactgactcaggcttagatgacaaggaagatattgaatgcttttttgaggcctgtgttgagggtgactctgatggagaggagccttgtttctctagtgctcctccaaatgaatctgcagttcccagtgaagctgcaatgccactacaagcaacagcatgttcttctgagttcagtgatagttctctttcagctgatgatgcagatacagtggctctttcaagtccttcctctcaggaaagagctgaggttggaaaggaagtgaatggtttgccccaaacttccagtggctgtgcagaaaacttagagtttactccttcaaagcttgacagtgaaaaggaaagttcaggaaaaccaggtgaatctggaatgccagaagaacataatgctgcttcagccaaatctaaagttcaagacctctccttgaaggcaaatcagccaacagacaaggcagcattgcatcccagccccaaaactttaacctgtgaagaaaatcttctaaaccttcatgaaaaaagacatagaaatatgcataggtaggagctcaggcctcacctggatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattaccactccctctctgcgcgctcgctcgctcactgaggccgggcgaccaaaggtcgcccgacgcccgggctttgcccgggcggcctcagtgagcgagcgagcgcgccagctgaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaatcagccagccggaagggccgagcgcagaagtggtcctgcaactttatccgcctccatccagtctattcattgttgccgggaagctagagtaagtagttcgccagttaatagttttcacaacgttgttgccattgctacaggcatcgtggtgtcacgctcgtcgtttggtatggcttcattcagctccggttcccaacgatcaaggcgagttacatgtcaccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagt

SEQ ID NO: 4 - DNA, 5707 bp, synthetic construct
  source 1..5707: mol_type = other DNA
cattctgagaatagtgtatgcggcgaccgagttgctctcacccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttcaattcaactactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctactcgagcctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcagtagaaaaacagccaagctagggaggctgggaggccaagccccagataccttacatagctctgctcagcctctgtctcattaggaactccatttttaggatgcagttgtttcaggctaaaaataaatcatgcaatgaataaaaaagttagatacgacactgtagagggattcgctgatacagtctgtccgatctagagagagtcagctcagctgcttactggcttgctttcctcctgtctctttcattttcataatctaccaaaccctgcaatgggccagccttgaacatacaagtgcatgtgcatggtcagacacaggcaagcaagcaagacccctaggcctgacctatgcatctgcaatctagtaggtttagcagatcatagccccgcactgcttgattttaaagccgttaggggatgacctttgacagtccgcatcacccctctcacacaacgagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagaactgaaaaaccagaaagttaactggtaagtttagtctttttgtcttttatttcaggtccctggtggtgcaaatcaaagaactgctcctcagtggatgttgcctttacttctaggcctgtacggaagtgttacttctgctctaaaagctaagcttgctagcaggactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgcgggaggaaatagcaaggttatagtgaagccacagatgtataaccttgctatttcctcccttgcctactgcctcggaattcaaggggctactttaggagcaattatcttgtttactaaaactgaataccttgctatctctttgatacatttttacaaagctgaattaaaatggtataaattaaatcacttttttcaattggaagactaatggcagcctagagctcaggcctcacctggatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattaccactccctctctgcgcgctcgctcgctcactgaggccgggcgaccaaaggtcgcccgacgcccgggctttgcccgggcggcctcagtgagcgagcgagcgcgccagctgaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaatcagccagccggaagggccgagcgcagaagtggtcctgcaactttatccgcctccatccagtctattcattgttgccgggaagctagagtaagtagttcgccagttaatagttttcacaacgttgttgccattgctacaggcatcgtggtgtcacgctcgtcgtttggtatggcttcattcagctccggttcccaacgatcaaggcgagttacatgtcaccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagt

SEQ ID NO: 5 - DNA, 5773 bp, synthetic construct
  source 1..5773: mol_type = other DNA
cattctgagaatagtgtatgcggcgaccgagttgctctcacccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttcaattcaactactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctactcgagcctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcagtagaaaaacagccaagctagggaggctgggaggccaagccccagataccttacatagctctgctcagcctctgtctcattaggaactccatttttaggatgcagttgtttcaggctaaaaataaatcatgcaatgaataaaaaagttagatacgacactgtagagggattcgctgatacagtctgtccgatctagagagagtcagctcagctgcttactggcttgctttcctcctgtctctttcattttcataatctaccaaaccctgcaatgggccagccttgaacatacaagtgcatgtgcatggtcagacacaggcaagcaagcaagacccctaggcctgacctatgcatctgcaatctagtaggtttagcagatcatagccccgcactgcttgattttaaagccgttaggggatgacctttgacagtccgcatcacccctctcacacaacgagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagaactgaaaaaccagaaagttaactggtaagtttagtctttttgtcttttatttcaggtccctggtggtgcaaatcaaagaactgctcctcagtggatgttgcctttacttctaggcctgtacggaagtgttacttctgctctaaaagctaagcttgctagcagccaccatggtggagctctctctttgctcagaggatattgtgttacacaagaacaagatcccagaatctaatgcatctttcaggaagagactgactagatcagtggctgatgaaagtgatgtcaatgtcagcatgattgttaatgtctcttgcacctctgcttgcactgatgatgaagatgacagtgacctgctctccagctctacccttaccttgactgaagaagagctgtgcatcaaagatgaggatgatgactccagtattgcaacagatgatgaaatttatgaagactgcaccttgatgtcagggctagactacataaagaatgaattacagacctggattaggccaaaattgtctttgacaagagataagaaaaggtgcaatgtcagtgatgagatgaagggcagtaaagatataagtagcagtgagtaggagctcaggcctcacctggatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattaccactccctctctgcgcgctcgctcgctcactgaggccgggcgaccaaaggtcgcccgacgcccgggctttgcccgggcggcctcagtgagcgagcgagcgcgccagctgaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaatcagccagccggaagggccgagcgcagaagtggtcctgcaactttatccgcctccatccagtctattcattgttgccgggaagctagagtaagtagttcgccagttaatagttttcacaacgttgttgccattgctacaggcatcgtggtgtcacgctcgtcgtttggtatggcttcattcagctccggttcccaacgatcaaggcgagttacatgtcaccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagt

SEQ ID NO: 6 - DNA, 5917 bp, synthetic construct
  source 1..5917: mol_type = other DNA
cattctgagaatagtgtatgcggcgaccgagttgctctcacccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttcaattcaactactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctactcgagcctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcagtagaaaaacagccaagctagggaggctgggaggccaagccccagataccttacatagctctgctcagcctctgtctcattaggaactccatttttaggatgcagttgtttcaggctaaaaataaatcatgcaatgaataaaaaagttagatacgacactgtagagggattcgctgatacagtctgtccgatctagagagagtcagctcagctgcttactggcttgctttcctcctgtctctttcattttcataatctaccaaaccctgcaatgggccagccttgaacatacaagtgcatgtgcatggtcagacacaggcaagcaagcaagacccctaggcctgacctatgcatctgcaatctagtaggtttagcagatcatagccccgcactgcttgattttaaagccgttaggggatgacctttgacagtccgcatcacccctctcacacaacgagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagaactgaaaaaccagaaagttaactggtaagtttagtctttttgtcttttatttcaggtccctggtggtgcaaatcaaagaactgctcctcagtggatgttgcctttacttctaggcctgtacggaagtgttacttctgctctaaaagctaagcttgctagcagccaccatgggtaaaagcagcactccattgccactagacaccactgactcaggcttagatgacaaggaagatattgaatgcttttttgaggcctgtgttgagggtgactctgatggagaggagccttgtttctctagtgctcctccaaatgaatctgcagttcccagtgaagctgcaatgccactacaagcaacagcatgttcttctgagttcagtgatagttctctttcagctgatgatgcagatacagtggctctttcaagtccttcctctcaggaaagagctgaggttggaaaggaagtgaatggtttgccccaaacttccagtggctgtgcagaaaacttagagtttactccttcaaagcttgacagtgaaaaggaaagttcaggaaaaccaggtgaatctggaatgccagaagaacataatgctgcttcagccaaatctaaagttcaagacctctccttgaaggcaaatcagccaacagacaaggcagcattgcatcccagccccaaaactttaacctgtgaagaaaatcttctaaaccttcatgaaaaaagacatagaaatatgcataggtaggagctcaggcctcacctggatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattaccactccctctctgcgcgctcgctcgctcactgaggccgggcgaccaaaggtcgcccgacgcccgggctttgcccgggcggcctcagtgagcgagcgagcgcgccagctgaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaatcagccagccggaagggccgagcgcagaagtggtcctgcaactttatccgcctccatccagtctattcattgttgccgggaagctagagtaagtagttcgccagttaatagttttcacaacgttgttgccattgctacaggcatcgtggtgtcacgctcgtcgtttggtatggcttcattcagctccggttcccaacgatcaaggcgagttacatgtcaccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagt

SEQ ID NO: 7 - DNA, 10227 bp, Homo sapiens
  source 1..10227: mol_type = genomic DNA
gagtctgggagctgagtgactgaggatttgaaaccttgctatagttacagttcataacaagcgtctaggcagttagaccttaagtgttcaggtatggaaagaaagtcatatgttatgttttagattctgtttgtaaagctggttaactagagacagctgatgaaaaaccaaatcgacttgagttacaagatctgggctttctctgctctgctttcaacctgttggttggtggtggagtagctgacagaagcgaatggcttggctgagggacatgaagtgacagcagcctgtttaggaccacaccacattttggacctcttgctgtgcagttcaggacatttgtgaagatatttctgatcatgttgagcaaatccatgccctccttgaaacagagttctccctaaagctgctgtcttactctgtcaacgtgatagtggacatccacgcagtgcagctcctctggcaccagcttcgagtctcagtgctggttctgcgggagcgcattctgcaaggtctgcaggacgccaatggcaactacactaggcagacggacattctgcaagctttctctgaagagacaaaagagggccggcttgattctctaacagaagtggatgactcaggacaattaaccatcaaatgttctcaaaattacttgtctctggattgtggcattactgcattcgaactgtctgactacagtccaagtgaggatttgctcagtgggctaggtgacatgacctctagccaagtcaaaaccaaaccctttgactcttggagctacagtgagatggaaaaggagtttcctgagcttatccgaagtgttggtttacttacggtagctgctgactctatctctaccaatggcagtgaagcagttactgaggaggtatctcaagtatctctctcagtagacgacaaaggtggatgtgaggaagacaatgcttctgcagtcgaagagcaaccaggcttaacactgggggtgtcatcatcttcaggagaagctctgacaaatgctgctcaaccctcctctgagactgtgcagcaagaatccagttcctcctcccatcatgatgcaaagaatcagcagcctgttccttgtgaaaatgcaacccccaaacgaaccatcagagattgctttaattataacgaggactctcccacgcagcctacattgccaaaaagaggactttttcttaaagaggaaacttttaagaatgatctgaaaggcaatggtggaaagaggcaaatggttgatctaaagcctgagatgagcagaagcaccccttcgctagtagatcctcctgacagatccaaactttgcctggtattgcagtcttcttaccccaacagcccttctgctgccagccagtcttatgagtgtttacacaaggtggggaatgggaaccttgaaaacacagtcaaatttcacattaaagaaatttcttccagcctgggaaggcttaacgactgctataaagagaaatctcgacttaaaaagccacacaagacctcagaagaggtgcctccatgccgaacacctaaacgggggactggttcaggcaaacaagctaaaaatacaaagagctcagcagtgccaaatggagagctttcttatacttccaaggccatagaggggccacaaacaaattctgcttccacatcctcacttgagccttgtaatcagagaagttggaatgccaaattgcaattgcagtcagaaacatccagttcaccagcttttactcagagcagtgaatcctctgttggctcagacaacatcatgtctccggtgccacttctttcaaaacacaaaagcaaaaaaggtcaagcctcctctccaagtcacgtcactaggaatggtgaggttgtggaggcctggtatggctctgatgaatacctagcactgccctctcaccttaagcagacagaagtattggctttgaagttggaaaacctaacaaagcttctgcctcagaaacccagaggagaaaccatccagaatattgatgactgggaactgtctgaaatgaattcagattctgaaatctatccaacctatcatgtcaaaaagaagcatacaaggctaggcagggtgtctccaagctcatctagtgacatagcctcttcactaggggagagcattgaatctgggcccctgagtgacattctttctgatgaggagtccagtatgcctctcgctggcatgaaaaagtatgctgatgagaagtcagaaagagcttcatcctctgagaaaaatgagagccattctgccactaaatcagctttaattcagaaactgatgcaagatattcagcaccaagacaactatgaagccatatgggaaaaaatagaggggtttgtaaacaaactggatgaattcattcaatggttaaatgaagccatggaaactacagagaattggactccccctaaagcagagatggatgaccttaaactgtatctggagacacacttgagttttaagttgaatgtagacagtcattgtgctctcaaggaagctgtggaggaggaaggacaccaacttcttgagcttattgcatctcacaaagcaggactgaaggacatgctgcggatgattgcaagtcaatggaaggagctgcagaggcaaatcaaacggcagcacagctggattctcagggctctggataccatcaaagccgagatactggctactgatgtgtctgtggaggatgaggaagggactggaagccccaaggctgaggttcaactatgctacctggaagcacaaagagatgctgttgagcagatgtccctcaagctgtacagcgagcagtataccagcagcagcaagcgaaaggaagagtttgctgatatgtcaaaagttcattcagtgggaagcaatgggcttctggactttgattcagaatatcaggagctctgggattgcttgattgacatggagtcccttgtgatggacagccacgacctgatgatgtcagaggagcagcagcagcatctttacaagcgatacagtgtggaaatgtccatcagacacctgaaaaagacggagctgcttagtaaggttgaagctttgaagaaaggtggcgttttactaccaaatgatctccttgaaaaagtggattcaattaatgaaaaatgggaactgcttgggaaaaccctaggagagaagatccaggacacaatggcagggcacagtgggtcgagtccacgtgacctgctctctcctgaaagtggaagcctggtaaggcagctggaggtcaggatcaaagaactgaaaggatggctaagagatacagagcttttcatcttcaattcctgtctgagacaagaaaaggaaggaacaatgaatactgagaaacaactgcaatactttaagtccctctgtcgtgaaatcaagcaacgacgtcgaggagttgcctccattctgcgactatgccagcatcttttggatgaccgggagacttgcaatctgaatgcagaccaccagcccatgcagctgatcattgtaaatcttgaaagaaggtgggaagccattgtcatgcaagccgtccagtggcaaacacgtctacaaaagaagatgggaaaggaatctgagactttgaatgtgattgatcctggcttgatggacctaaatgggatgagtgaggatgccctggaatgggatgaaatggacataagtaacaagttaattagtttgaatgaggaatcaaatgaccttgatcaagaactccaacctgttatcccttccttgaagcttggagagacaagtaatgaggaccctggttatgacgaggaggctgataaccatgggggatctcagtatgcctcaaatattactgccccctctagtccacacatttaccaggtgtacagcctccacaatgttgaactctatgaggacaaccacatgccatttctgaaaaacaatccaaaggtcactggcatgacacagcctaatgttttaactaagagtctcagtaaagactcttcattttcatctaccaaatctttgccagatcttctaggtggttccaatttggtaaagccctgcgcatgtcatggaggagacatgagccagaattcaggcagtgagagtggaattgtcagtgaaggagacacagaaaccactaccaactctgaaatgtgcttgctcaatgcagtggatgggtccccaagtaaccttgaaactgaacatctggacccacaaatgggagatgcagttaacgtgttaaagcaaaaatttacagatgagggggaaagcattaagcttccaaatagctctcagtcgtccatttcaccagtgggttgtgtaaatggaaaagttggagatttaaacagtattaccaaacatacccctgactgtttgggagaagaattacaaggaaaacatgatgtgtttacattttatgattactcatacctccaaggctcaaaactcaaattaccaatgataatgaaacagtcacaaagcgaaaaagtgcatgtggaggatcccctgcttcgtggtttttattttgataaaaaatcatgcaaatctaaacatcagactacagagttacaaccagatgtacctccccatgaaaggattttggcaagtgcatctcatgaaatggatcgcatttcatataaaagtggcaatatagaaaagacattcactggcatgcagaatgccaaacagctctcccttttatctcatagttcatctattgagtccctttctccagggggtgatttatttggattgggcatctttaaaaatggcagtgacagcctccagcgaagcacttctttagaaagttggttgacttcctataaaagcaatgaagatctctttagctgtcacagctctggggatataagcgtgagcagtggctcagttggtgaactaagtaaaagaacattagatctcctgaatcgtttggagaatatccagagcccctcagagcaaaagataaaacgaagtgtttctgatatcactcttcaaagcagttcccaaaagatgtcctttactggccagatgtcattggacatagcatcttctatcaatgaagactcagcggcatctctaacagaacttagcagcagtgacgagctctctctttgctcagaggatattgtgttacacaagaacaagatcccggaatcgaatgcatcgttcaggaagcgtctgactcgttcagtggctgatgaaagcgatgtcaatgtcagcatgattgttaatgtctcttgcacctctgcttgcactgatgatgaagatgacagcgacctgctctccagctctacccttaccttgactgaagaagagctgtgcatcaaagatgaggatgacgactccagtattgcaacagatgatgaaatttatgaagactgcaccttgatgtcagggctagactacataaagaatgaattacagacctggattaggccaaaattgtctttgacaagagataagaaaaggtgcaatgtcagtgatgagatgaagggcagtaaagatataagtagcagtgagatgaccaatccctctgatactctgaatattgagacccttctaaatggctctgtaaaacgtgtctctgaaaataatggaaatggtaagaattcatctcatacccatgagttagggacaaagcgtgaaaataagaaaactattttcaaagttaataaagatccatatgtggctgacatggaaaatggcaatattgaaggtattccagaaaggcaaaagggcaaaccgaatgtgacttcaaaggtatcagaaaatcttggttcacatgggaaagagatttcagagagtgagcattgtaagtgtaaagcacttatggatagtttagatgattcaaatactgctggcaaggaatttgtttcccaagatgttagacatcttccaaagaaatgtccaaatcaccaccattttgaaaatcaaagcactgcctctactcccactgagaagtctttctcagaactggctttagaaaccaggtttaacaacagacaagactctgatgcactgaaatcatctgatgatgcaccgagtatggctggaaaatctgctggttgttgcctagcacttgaacaaaacggaacagaggaaaatgcttctatcagcaacatttcctgttgcaactgtgagccagatgttttccatcaaaaagatgccgaagattgttcagtacacaactttgttaaggaaatcattgacatggcttcgacagccctaaaaagtaaatctcaacctgaaaacgaggtggctgctcctacttcattaactcaaatcaaggagaaagtgttggagcattctcaccggcccatccagctgagaaaaggggacttttattcgtacttatctctctcatctcatgacagtgattgtggggaggtcaccaattacatagaagagaaaagcagcactccattgccactagacaccactgactcgggcttagatgacaaggaagatattgaatgcttttttgaggcctgtgttgagggtgactctgatggagaggagccttgtttctctagtgctcctccaaatgaatctgcagttcccagcgaagctgcaatgccactacaagcaacagcatgttcttctgagttcagtgatagttctctttcagctgatgatgcagatacagtggctctttcaagtccttcctctcaggaaagagctgaggttggaaaggaagtgaatggtttgccccaaacttccagtggctgtgcagaaaacttagagtttactccttcaaagcttgacagtgaaaaggaaagttccggaaaaccaggtgaatctggaatgccagaagaacataatgctgcttcagccaaatctaaagttcaagacctctccttgaaggcaaatcagccaacagacaaggccgcattgcatcccagccccaaaactttaacctgtgaagaaaatcttctaaaccttcatgaaaaacgacatagaaatatgcataggtagaatgtaccccctccccaagcatgaaaatcatctcactgaaagatacgcctggctgcaactcaggggtggcctcatcctcccgccctgggctggcctctggttccatcacgtttgtcactgccgtttattacattgacttctcccaagatgaatcttccttccaaatgtgttttctccacacaagccttgtgatctgaatgtgtgcgctggttctctttaggtgatcgtctttgaagttcagcaaagctgcttgttctcccatggattcctgtcccaagctacctctaccaaccctctctctccagctagacttttctctttgcctcctcccttcccttccactctttaaagttctgcagttcaccaactggtagtccattaaattctcctgtctagaatgacccccccaccagtacttgaccaatttcatgtatcaatctggattttttttttaacggtataatgactgtgcttattgaaagagttttacctaaaaagccaacatttgaattggttgcagcatagagaagaaacactggtccttctttcaaaattaagcaactattaaaagcgccattttatttatttcatttaaaaaataatctatgcagcatttcaagaaacaaccatatggtgttgtatattataaactggtgacattctactattgaattatgtacaacattttcattttttatgcttcttgaggtggtaatgagaaaaaagttttttaaaaaagtgtgccttgctgtatttcttataccatttattaaaaagctgctttcacggtaaaattatgttggtttgaaaggaggaaatagcaaggttaagatgtgtgaataatttctgtatatatgtataaccaagtacaaacattgatgtataatgacagtataaaatgctttcatgtttgtgatgtctagtgatgtggaaaatataagccttaaatccattagattgcatggtaattaaaattggcataataaacacagattattgggggaaaaggaaaattagtgatctcttctactatgttctttaccaaattgttgcatctggttctgaaaaagtatagcatgtagcagcttccaaacatattcatattgcttaagaggcttaacattacctaaactagagactagacgtaaagccttcagttttcaaaatctttctggtcactataaagatcttggaacagcaaatgattaaatgtcagttcccctaaaccaataaacatttatactagattttttatttccacttatcattaatgatttaatgttggatttcaggtaccttgtatgtcttaatttattttaaatatttattttgaatgagtttgatagaaagctagtagaaaagtacagaaaatttgactattatttatagatttcaggtatatttatatgtgtaaaagaaattgacaaagaaatatttcatctggcctttactgactcctgttaaatgcagttttaaatttatatcgtaacacctacttaagtgcctgacacagtaggtattcaataaaaatttactgaattaaaggattaaattaggtgacatggtgacatctatccctttattttgacactaaaacatggacacaactagaaagaggtacaatgcaatataaagtcacaatagataatatatatcaaatttctaaaaggtaaagaatgttgtgggttcatgcagtcacaggaatgacaatcattcaacagatagttcagaaacactttttatctgcaaggcactattctagatccagaagatgcaatgttgaacaaacagacaaagccctgccctcagaaggctgtcctgcattaggaacaagtgaacacgcaaatgacatgaagtatttgttgcagagctgaggaacagagcaaatgtagtgatagaagcgcaatgagagaagcagcagtgggtacaaggaggaagaaaaagggcttgcagagagtggaaagttagtggaatattcatgaaacttcattgcaggggtaatagaagaaaaagtaaattgggaggacttaatggaaggtcttttaaaaagttaacttggagcttctgtatgtaaaatgctaggtaataaggacactttgtacaggctgttttgcacctgattttatttatcattagtgccacgccaagatcatttagacgatgcttatctgtaattctaccactttaataactatttgtatttttatgccccttctgatcttttccatatgtatttctaaatggataaattattctaggcttcttaataggtagtaatttgttcaaaagcggttttagccagacatctagttgcagtgttcaagaggattatgggggaaagagattagagataattgtctagttagggggcagctggagaaaataagctaagtttgcaataacagagtacacaagtatagtggcccaggatgtagtgaaagaacaaatcctagagtctttgaaatttctaagggcattctagacctctgttgggatatggtattattttacatactgacacaacctaaattttctttgggtagtaactaatgtcaagtctacatcgactggtaaaacattcaaagaacaaactgacaatgatgttctacctacttgttacatgctcatggaagaccgtgcagtattgaaagtatttgttaattatctgcttagtattaacactaaatttgtagaatgactttcaggtttgttgaacaatgccttttcaggttggaagaagaaaaatagcctcaatctcccaccccatgtaggcactacctccccaattacccttagaaaatgatcacaccaactctgcctacacacttccagtgatagtggctcattgtctgttaaggcaaactgttccactgttgggcatatctctttgttagaaagttctttcttaggttgctaaaatctgcctagtaccccgctaccctgttctgtcttatggagcagcccagattatctttactccctctttctcatggcaaccctgaagataatcaaggccagttactcatcatctcccaaccactgtttcctcaactgcccttcatatgtcatggttttcagatccattccaacctgactgaatgttaacagacagaattcttcacattaaggaactgtcttcatcatcatacatgtagaaaagaatctgaacatttaagtgcgaagttttctctagaaatatattcaagatatgtttattctattattgtaaatttcaaacaataaataaataagaatcc

SEQ ID NO: 8 - DNA, 25664 bp, Homo sapiens
  source 1..25664: mol_type = genomic DNA
ggaatctggagatcaggacccccaggtatcctcaccctccctgatgtctcttactgtggagcctttgtggtgggatgactggggacagaggagggacagtcgggagaggaggagtcatttcgggagaggaggagtcatttcgggagaggaggagtcatttcaggagtggaggcagtcgggaatagatgggtgtgtgggtcagactctggcagagaggaggggtgggtctgggttccttttctcttttgggaagagaaaggacatcctgggctgggagctgggcctggggtatcccgagccctttggagcctggtcttagtctctggtctccaccctgataccctcaatcaggaggcacagaaagcatcatctgcaaccgagaggactcagtggggacagaaatctgactcctcgctggacgctgaggtgaatggtggggaggagcctggccctacggcggagggccacggtgtactgtgggagaccccatcgtccccgtgtgcacacactaattggcctgcaatcgcgcagtccacccagaacgcccccatccctgctcatgcagattggtctgagccaaccgtgagaacaaggttgagccctcacctggttgtgctgcctgcagctctgcagagcccctccctcttcagctggcagattacagggctctgactccttgtgggtgtggggcttgggcaaagggatgccttcctatccagtggcccccttccccttccatccctgacaccccattgacttgtgctaacagctctttgccccacaggtgtgacaagccccgccaagacagacctgcaagtcttcgtctcaagggacctccctcatgccaggcccctgcctctcacagcagcaccctttcctctcattgtccctgttccctttttgcctgtggatctgtttggccagggtccctggggtcaggaatatttgcaagactcagccagctccttcccagcccagcctcttggggctgggactttctcaccctgcggcaggcacaacagatgctgggacccagtctctgcccaggtcacagcacaagtgcacatcagcactatggggcctatgtcctgcccagagacctctgctccttcctgctcacatccacagtcagggcacggcgcccctcaagaactccagagtcacctgtctcatcggctcccagcaagtgcctctttgtctatgatgtcccccttctctgaggcctggacccacccatctttgtccctggggcctgctcccagccactgaggcccgctctggccaggggagaaggagctgccgtgcgtcttccctgtgccccgtctccctgcttggttctcccctcccttccctggccggctgccatggccaggagctaagtgcctttttgtgtgcaaccacttaccctttctctgaaaaacctgttctcaggaaggatctgataaactcatttactctcaggtgtaagagactgatgagaccttagaagcgaattcctctctggaggccttgctttctagcagaggtcacctgaagtgtgtgaggaggatcatcattttcctcatcccccctcttctcacattaaggtggtggcttgccactcagcagtcctagcttggtgactgggaactgccacatacagggccaggcctaccctccttccccacaagccccctccaacccccacccccatgctctggacctcatggctcctatgagcttggagcatggtgaaccatcagagaatctagaaccaaccaagctaggaacatcagcctggtgccctgttaaccccttaaagctgtggtttacaacttttcaaaaatttaaatcattagaactctccatcttgattgactgccctagttttacccagaatcagctggggatgtggccaggtgcagagccccctgcccattccccctttgcatgcctggggaagctccaggtctttttggaacacgacccttaacacggctcctaagggggtatcaccagtccagttccccaagtgtgtgggggatgagccctagccctactctgtgactctgggtgagggctcctcagatctgtgccccaggggacaggccctcaaagtgccatccgtgctcaggtcccagtgccccaggctgacccacctctggtcagtgagtctcctgtgcctgagtgagggatatcgggacctggggtccctgccccgagcacctcccacccactgctagtgctggggctttgtgagtgttccaacttcatagccgagagttggaggacaaggctggggcagggccgaggaacggattgagtcctgcctaagcctcgggacatctaaacagctctggctctgcccagacctcaggtgtgaccctgagccattttccttctctgaacttcaagcagatgacctcagagaactttcatatgttcatgccatgataggtgaacttttccagtaaacaaaactgtccccacacagaggagaggctcctggaggccttcacctggcagggcctgaccgtggagtaagagcctaggaagacactgaggggcctgttcctgcagctgcagcctcagcagggcccttcatgcctgcacccccctcccctggggtgcccagtgagggctgactgctaggggaaatgtcggggtgctacagcacagccccctcccctgtgaatccagcacgtgagggccctgccctcgttcctactggggacgcagccagtaaacaaggagaccagggtgtgcccaagatgacctcagctggggatagatgagtggtagcgactgggtgtccaggcgggtggggaggcctctggatgggtgacattgggcaccttctggagacagaacagggctcccacacaccactccgggctgcaccctcctgctgtcttgccacaggggccctgtgccccacaagtggcccgtcctattgtggatcctcaaatttacatgcactgtttcagcataagcctcccattccactctggctttgttttctctttacatccctttgaaaggacagggatataaaaaaaaaatgggctaagagtaaccctactatgcccaggagtggctgctgcatgggggctggatttccttatctgagaggctgtctcacagcgctctgaacgaagctcctgccttagaacaattcgaagccatacacgtgtggcctgtgacaccaaggatctactgggcttttcccatggggctcagtagtgacctaggtgcccagatccctcttctaggcatccatgcaaataaaccgtggtctgtttgaaaggttattgggcttacaaggaacagtaacccaccctcaagttaaaaaagggtttgggaaaagtaattccgaattctacgttactccaggccaggacccaaggcaaggctgggccctgcttcgtgggctagaatgggagagagagatgatggggctccccccactttcttgacagcctgactccttcctcactatccctcgggaatctgtcccctccatctgcctgtctctctacaaagtgcccctgcttccctgtggtggtcctcagtcccacaatgctggccagcgggcggttggcttcagcttgccctgccacagatgtgctggcagtgtggaggcccacgtggcagggcaatcaaggccactggactcagaggccacagggaactatggaggggatggagtcagctgagtgatgggacaagagtctgacagcagcatgggtgtggagcaaatggcagtcccctgcgcagaggaccctttcaaggacattagtggtggaggcagcatagtagctcccaaggcagagggattgagagaagagtttgaggactgggaaggcgggacacatgattgggtgatgggagaagggggcagagaatagcgagattgctttctttgcccacggagaaacagaggagtgtggatcatgaatgggcaagatctttaagtgccagggggggtcatggaggaggggagggcctgctccagaggaggaccattcctgcttcagagccaagcaggacctaggctgtgaagattcggagaaagagatggaggggagagtcagctcagctgcttactggcttgctttcctcctgtctctttcattttcataatctaccaaaccctgcaatgggccagccttgaacatacaagtgcatgtgcatggtcagacacaggcaagcaagcaagacccctaggcctgacctatgcatctgcaatctagtaggtttagcagatcatagccccgcactgcttgattttaaagccgttaggggatgacctttgacagtccgcatcacccctctcacacaacgagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagctgctgttctgaggtaaggctcgggcagggctctggggaagaggagagcagagaatggacggggagatgtgagggtcttgggccctggcatatttacccagagtctgcctgtgtccgcagaagtccatggcccctcctggtggaggccacacttcagaggacaggttgccaggtctgggctccaagattggtacaatagagcagagagaaagagagaatcccatggtagccattggagactagggttggagtcctggacctgccactaaagatgtagagtttaaactcttggatgagtcactttccctccccaggcctcagttttctcatctgtagagtgggctaaatatttgctaggcctctgcctgctctaaatacaatgaggctctaaccaacagagactcccctaaattgcttgttccaggccgagggtcacctatacctttccagtcaggtccttcttgagtcccagggagcagagcgagactgcagccctgtcagtcaagtgagtctcctggtcatattctcaaggctaggaactcagaaactgtcctggaagtactgagaacgaccagatgtgcagggtcctctgcagcgcctctccaggccaacgcgggaattccatttgatgccgctgactcagtgccattccctcccagagctgagctgagctcccagaccctgagctgagctgcacttggggggcagatggggaaaggccccactgggagaacctgtccttgggtgtgagcccagctggttggcattaaagggtttaacctgggcccccgtaagtgtaacccttgtcccatcccaccccaacaccccatcttgggacaagccttgctctgtgcccaggagtggtagagaaagattcccttggtttctagtttctcctgggcaggctgaggactattcagggagggggttgcctggtgtgtaaggaatttcagagcacattcctggcctgctgtagccgggaagctacctgccctcccaaattccttttctgacccatgtcttagagggcgaatcttccttctctctgcaaaagtggaatctgagaggatcaggttgctggctgcctcctatttctgacccccacttccactcttcctgccctctccccatccccttacccccgctctcctggtgggctaacagctgttggcttaggaaaatcccattcgagtgctggggacaaggactgaggggtggggcctgtgtttctcagaaactcggagactgtttctacagggacagaactcaaggctctcctgctttgtagttggttaactgaagtcagagaggctaggtgacttaaataaggccacacagcaaaatctgggttttaagcccagattttttgaccccaaggtcagtatgccttatgccttctattgtctttgctttactccccaagaaaggtgaccctgtgaacgtgttaggcttccaacacgtgttggctatgaatgcattaggctagaaagctgcctgtggaagtgggtgggacttgggtgtgaggacacaggtgtttcaggacattctggttccttgccctgccaggttcttgactctctcccattctgcagcccccagtctcagcccacctctttcccctgcatcaggtcctgcctttgagccactggcatggtggtgtcctctgggctggctgaaagccctcctccatcctgcatgtctgcatgctatttttgggagctggatggtgcccccaggaacagggagaggagtccaagctgctctgtcctcctgtgaggccccagcaccatgcccagtggctctgaactggtggaagggcagagtaggagaggagagagaaagctagaagctatgcgcccaggctggccatgcacggttcctctgagaattttctccctcccactgagggacaagcttctcctggggctcaggtaggagctcctcaagttctcaagtcactagttctggccgctcccctggggctttgggtaacaattgtcagccccacccatcctagggttaggaaggctattctctttggccactctcatcctaagacctatttggagaacctctggggtttgagtctttttttcagcagaatgaggcttgatcccgcattatagcacctcgcacatttgatgtctcttcttctcacccactcaccccaccctgggggttggggcaaaaaagtggctcagagctgcggttcagagctccttgtaaacaaggctcctccctcactgtcctcaccctgctccagcagagggagcagcggaaggaccactctgctgcagccatgcttgtttctaacccagcagaactggacataatgggaacagggtctgaagacaatcaatccagggctgcagtgggtgctgagtctggggaagcctccacctggaggggcagctgggcagtggcagctcccttggaatggctcagcctctggacatcaccccacccaaccagagccctggctcttgctggatgtccacagatgagtgcctgggattggtctcagccactatgggggggatgtgcagggagaggtgatgagggagtgagcaggactgtctatgtgcctctgtcctcatcctgaggcttgggtctgaaattggtgctgcagcactggcacgcgtgagtggggcaggaggtgggcagcaggggaggagcattggagggtggtcctgagcatcatttggattcttctcccgcctcactttagcctcactctcacatcaccctgtccattctctcccttggaagccaacatcaggaaggctgtggagctggcatattgcactataacagaggctgctgttcacgtttcaaacaacgtgattgtgtccctggagcagtgcactgtcatggccccggagggttgggtcatagtagttattagctgaatgcctacacttcacactacctctgttcctcacacctacccaagaagagcagtaatttcacagcaaagagagctgagatgtggggaggttaagtgaccttgccaaggtcgcaggcaaacgcatgctggagccagcattcaaaacgagattagtctcacactcaagtgcttgctctctgccccctccctgtgattacatgctcattgtaacacctgatgttgcctaatgccttccccttttgttaatgtcaactggagcctcgtaaagaccctgagtagccaggatgtgtacactttatctccatgagacagagaaaggactgaggtttggagatgttaaaaagactcgcctaaacaccctcggcaagctaatggaagaggatctgaatctagattatctgttcccagtgcagtgagaagatgatctcagaatgagttccctgggggaacattttttaatcggaattttcattgacataattaaagactagtacacagttgtctttaataacgagcgtttacacacggtgcctagtttctcccgagggcaacatttgcaaaattagtactatatcaaaaccaagattggacatcaatacaatcaatctatgatcttattcagatttgcccacaattgcaccatgtgtatgtgaaactctaaacaactttaacctgtggaggttcacgtatccatcaccacagtctacatactgaaaagatcccaaaccacaaggatccccgctgtgtgctttgatttatttatttagagacaagttctcactcatttgtccaggccagtctcttggggtgcccaaagaccttttgtccaggtctcacttttgtccagcacacatggaatgggtgtggtggctcacgcctgtaatctcagcacttcgagaggctgaggcaggaggatcccttgagcctgggagttcgaaacagcctgggcaatgtggcaaaatcccagctttactaaaaatacaaaaattagtcgggcatggtggtgcacaactatagtcccagcaactcaggaggctgaggtgggagaatcgcttgagcccagaaggttgaggctgcagtgaggctttgtgcagtgaagatttgtacagtgctgaacagatcctgtggagtgggggtgggcagagtggatggaaaggttgagaaactgcagacattgtcttgagtgtcacaaatggaaatgcagcatcccaggcagtattgcctgttcaggggcaaggaacgaagtggacatcagcaggtggccttgctgccatgtgggtgtcactatctcccccagcaggggagaaaacaggctttttgttgcaggtcacacagctcatgaggggtggaactagattcaccctaggcctcgctgatctctgtacaacgggggccagagctcttctgaggaaggcaggcttccctttgtacctgcactgacttttttctccttttggagggagagcagagaccatgtctgacatagaagaggtggtggaagagtacgaggaggagtgagtatctggagcatcttgcctgagtggggtcctctcccgccgctgccctgacacctggtccaggagcctcccagctgtccctcggattctgggtagaagtagctgtgtgtgttttgggcaccccgaggagagatatagccatttcctttccactcctgtacccctgggttgtagatcccaggcagagcacaggccgagagacttcgggaagcacagaggttcagctccagggtcatcagtgtccttggccagtcttggctaggggccatgaacagagaagctgagatgttgggtctcctgaaactgactgaggaaggttcagtgactctccagaggcacagagctgattatgccccccaaaccccagctcagaacccagtgttatcaccctgtcttggatccctactctctcatgacagtgccctgcccacagtcctatcccagagggaaaccagggggtgggacacagccaatccaataactacagaggatagtggtcaccagggaggagacagaggtataggcacggctactgccctcacagaacttactcttgttgagatgttaagacataaactgggagaagaaattagcaactgggggcctggtgtggagggcgggctgttggtagtgtaggagttctgaggagagggagatcaggggctggaatagtcagggggctgtgtggagaaggcaggtcttacctgtgtagaatttggaaagataagagatttgggagagggcaggactttctagaaaggggtacagcttgggcaaagatggaaagagtaagtgagcagaacgctggtgagaccaagctggctggggcggaggcttgcggaggatggtaggaggcagccctgcatgagaacggcaggccaggctagtgggtgtcattgcaaggtgggcagggcagcgtggactccactaggcaacaagggaaaagaaagggggattatctttggggaaaggccagtgtgtgcatgtgtgtgcaggcgtgtgtgtttgcatgtgcttgtgtgcgagctactgacagtgtttcctgttgctctcagggagcaggaaggtaagcgtaaacgtgtgtactcatttggatcaaagacagcctggttcgaaactgacccacctcttctctcttctcttgctgcctggacttctcttctgagcagaagcagctgttgaaggtatgtgcctcaggcaggtggagagccagaaaggggcaggctcagcaatgtggaaatcctgggagccctgggaaagcagctcatctgtccctgctcctcactctcagctgagcaagatcctccaacaacagtgccttctaaaatgggaatcagtcctacctgggttgctcgggtcttgaggcaaacgatttaaacctgtctcttctgtctgccatttgcttcaaacacactgatcacttttcccaattttttttgcttcttctcccttttatcatgggaaattttaaatgcacataaaggtagcaataataataataatggccccccctgcccaaggtacccattaccagcttcaacaatcatcatcttagggccagtcttctttcctctatactccaccttccaccagttattttggagcagacatccaaaattgttcccaggcaccatgtcagttcacgcttcaagatttcgctacaccgtatctctaagagataagcactacttaaaaaccataaccacaatgccatgatatgccccaaaagattccatttagtaatttctcaaatgcgattgtgagtatgaaattgctcagtgatctgagacgtcttatctcggcatgaagatttgtcatgattctgagcttgcagaggcccctcttgctgctcctatgctgatcggttaacccatcgccctcctcagctgtgggtcccacatctccctgaaagaacgccctcaccttcccttcttcctgagagcttggcacatggaaatgactcagaggcttcactccagtcctccccctgcaccccaggcaggtggaccctgccttttagtggctcaggtatcacttcagcatgtggcatgtagatattaggagaagggtctctatgaaggtccaaggggggagcccaggtggccccgcccttgctccccatggctcctagggaaaatggggaccctcctgcacatttagggctgttccagggctgtctggccaaagaggagacagaagagaaattcacaggagaaactgaaagaggccacaggcgtcttttttggtttttaaggaacaccagatacttcagaagctatcagaaatatatttttgaataaaaaatctaactggctccaaattctgctgtgttccctgaacagactcttgatatgatatcccttcctttgttttggaggattttccacttccttataacaggtttagggtggtggagtttgagacatagatctctggatttgggaccaatctgcctgccacacacagtagaatttggctgtgagttagcagagtcattctccactgtgctggggcctgagccgtgtagcatctgccttgcttgccatgaatagacgagacttgcctcccacacagttgaggatctggctgcaaatgatcaagagagagcaacgtctagatgggtgtccactaagcctgcaggatagataatgccaggtgtccttcttccaggagaggaattgttccacttgctggaactgtttttttgggcagatatttgtacgtagaacaactgttgtagatgcagcctctttccttagatttataatgctggacccttcttagatggggcagcatcctcccaacaggccgggcaaatctcccctggggccaggctctccaatgaggctttctaaagtgtgactcctggaaactggacatagtttgcaggatacccaagagcctcattcagagcaggaacatgggcaggctcgttgagggcaggaaatttgatggaagagaattgtcccccactctctgctctttaggggcctctgtcttagtaagaaggaggccaaggctctgtcgtctagaggcattgcagttaccacagatcgcatgctcctcttgtgccaggcacctgctgggcgctctatgcacgctaggtcattcagtcctcaccacggctgagaagtgagtagaatgattcccattttactgatgaggcacctgaggctcagggaagtaactgagagttgcccctgttgctggaaagtagtggcttgggaattggaatgccggcctaactccaaagcccacactcttaaaaccactgcgctgggtggctgctcctgccgcgggctctctgctcccagactaacctgtctcgcttttcccctccgctgcggccactccctgaacctcagaagaggaggactggagagaggacgaagacggtagtacagcctttccttctgtggtgctttctgctgcctgctgtcccaagtgcagcctccttgtccaggggccctgttctgggggctggggggtgtgagtaggcggcagggacggagtgggtcagtcgtttcctcccctgcctcccaggggccaggatcacagttccaagctgatcatcatgcctgaacttcagggccactggctcctcttcctggttcctggatccttcccctgggttgggtggtgggagggaccaaggctagctggtctcccaagctgctccaaatgtggccatagaggggacctggcacctggaagacagcagcgccaccctgcccccgcgcacaccccagtgttccatgctgggccctccttaggagctggacagcaccaagcagggtggccaggtgttggttggggggtctggggacagagtcctctggagagcagccagggagactggaaatagccagagcaggaaggacatgacgtcagccttcagatgcgccctgctgatggggagcacaggaccaaggcaagggagtgagaccagggcttaattttagaaagtgcgttctgacagctatatctcaagatgcagtaggtgggttggggaggcagtaagctccccatccttgggatgttgcatggccactgggtgggctggggtatattatatagagaaatgacacgtcagatgaggtggcagaggtgtgaacaagatggcctaaaggtagaccccacaatggccatgtccttcctcgctagccctctgctttcacactttctagaaggatcattttgaggtaagactcctgcagggatcaaataaaaggcatctccaggcttcagagaggactcagacctgggaagggaggacagagggaatgaaggccaacccttctcccacgatggtgtcttcctcctgtctgaggtttttcttctccctctgccaagaggcacccacttcccagtgcatcagtgaacaggcatcacagaacacttcctacaggcacagcccaggccaggcggcatcctgtctcccttatctgtttgtctgtctgtctacaccaagtgcctgagtgagatgggggccacatcctgccaaacaaatagatgaaaatagaaaaatcctgcccactttctggatagtacgaagatccagatattgagtgtgccatccagccacacagtcgtactcttttggtttctttgagatgtggattgagggtgtctagagtcactaggttcaggacaggagcttctggacacctcggcttaagcagagcatgttctgtggtgccagacacggggtggaacttctgggctggaaagttggggaatgaggtggagtcccatcatcagggaggttgccgggatgagtggggctaagctgggtggcgggaggtagccgacagtcaaaaccaagaatggcagccccccagcacgttggttgcccccaaattcatctggacctagacgtcttgacccccatccccactccccgctgcccattctctgctctgggttctgcctgatagcatgagtggggcctgcttcttcactctgtctgcttcacttctccagagcaggaggaggcagcggaagaggatgctgaagcagaggctgagaccgaggagaccagggcagaaggtaagtctctggttgagggtgcaggggcctcagggggtggcgagagcccctggagccattcctgaggcagtaagaaaccccccagaacaggctttcccatgtgcacctgactctcctattgactaagattcaaccattgattgcccaatcaacaaacgttgacccagcgcttctcttgtgtcaggcagtcttgggtccaggggaatgtgtgtgtgagagacagctctgtaaccccaaaaaccaacagagtgcatcttggctagggcttatctgtggggaaggctcttgctggtggcggagggggggacttgagctcagaacaaggaccttgattgatttctcgtgtttcatttagaagatgaagaagaagaggaagcaaaggaggctgaaggtaaggatatcaggagagaggtacaagggaggcaaaggaacccctcacagcagctttggaggagaatgcccacagtgctctttccagggaagtggatgtgcactgagagggtttgggagaggagaggacagaacttggtggcacttccaaagttttagatgttgagctttcaactttgccccttctcccagagaagttgcattggtggcacccagtgggagaggggaccattgtccagccccctggtctggacagagcaatgggacccagaagtcatatccacacatgaagctcctagagcttaggtgaaatcacttgtaggatgtggtgtccgtaggatgaaatcccaatgcatagatggtcatggtgggtcattgctgcagtcatcgtcatgctgcagccttcaccagggttctaagtggtcccagtcacttctccaaaggggaggggacaaccagctggggcttggaatcatgtcatcagcttctgccctgtggccaccgggaagtccaaggggtaacaaagaaggcaaagacctgggaggggattctagggccaggacccttggggacctttccaaaggccttgtcactgtgaagcacaagagaagcaaaattgctttgcccagaactggtgagtgagagaaggctttgtgtgaagttccacactgcgtgagtgtgtgccctgcagaactctatgggagggggagtgtggcatctgcagcccccgctgccttcattctcctggatgcttctggaatcagggctccccagtgccgggagggactcactgtgcagatggggaaatggaaatccacagggatctagctcatcgagggcggagttgggaccacttctggggccccctgacccatggctgtgtgcttctatctcctccagatggcccaatggaggagtccaaaccaaagcccaggtgagtggggaggcacctgggtagagccgggagcagaggctcagggagagaggatgatgatggaacggggtgcagtatggtgcagtggacacgggctgggggtttggtgggcacagagcaccacagtacagttgtacaagttgtgtactgcacaagcgtctcttcctaagggagtgagtgagggctgaattccacccaacactccatcaccaatcgtgcaccctagcagggatggggctatgttcttctagaggaagggccctgaacggacactctcctctctcacagtatggccagaacaccctcctccatggccgaggtgggaccttgggattaagggaagcaaattgtggggagccaaacaatgaaaccgtgccagcataggcatggcggcttcagagagctgagagagggcaggacggcttggtgccacacacgtgaagcccacctgaaggtggtgtgtgctggggggggctgggggatagggagcatttccctttttatgatgccaaccctgccagcagaggttgacccatggcacctctgcccactcacaggctcttcccatttcagtggggccttctgaaaccagcccaagacatcccagatccatttatttgcccagtgccttcccccttcctggcctcttgtgcactgcggtatctcatttcagctccacacacctgagggcagatggggacatgaagaatcagagaaccccctgggtctcagtatgtgttgggaccaggaccaaactgcaagactcccaggttcatggccagtttctgcggggtccaccaggggatttgaagtccaagaagcatggcatcttgtcacagcttctttgattccaagttgtgtggctttgagcaagtcattctacctccctgagcctcagttctcctaagtgagattaacggtacccacttcatgggcatatcatgagcatgaagtgaaatagcatatgtgaaagagctttgtgtattcccaggtgctgagcaaggctgaggactgccattgttgacgtcagtgttactatcattgctgtggttggtcggggcagtgctggaagattctctaggaaggatcagggccctgcctgtcctggacaccctcagtccctgggtccagaatggggctgatgctgactattcctctctccaacaggtcgttcatgcccaacttggtgcctcccaagatccccgatggagagagagtggactttgatgtaagcggtggctgtgggttgagtaggcctgggctggggatagtcccaagggccctcagtgacagagattttgtgaccttgggctggggtagagcataggtgggatggggtgcagcaccagccaggctgggtgcccatccatgagcgtggtcctgggttcaggccacagttaccccttccctggccaggcgccaggccctgccagaggtcttttgcactgcgttggggggtgtctagcccacccatctctcctctggactctttggagtggcagcctctgagccgcagcggtccacccacaggacatccaccggaagcgcatggagaaggacctgaatgagttgcaggcgctgatcgaggctcactttgagaacaggaagaaagaggaggaggagctcgtttctctcaaagacaggatcgtaggtgtccagtctgcccacctaacattctctgatggcccagtctgtcctccatcccttttgtgaggccccacctcctccagacagccttcctcggcgcctcaaacccagctaggtgtgaggtctccttcagaggccctccagcattctgcacagctcctgcagccctgactggtttctcgggtccctgccttccctccccatcctactggatgctccttgactgccagagctgagtctgatctgtttcaggcctcccccagggcctaattcaggcccgggctgtggactgattggtcacaggcctgctgcgccacccctgcccttgcctcctcttcccccgcagatgaaggggagtgtgacctgcagaatcccacatccttctcctgcagagcgtggaggctgctttgattgcttttgtttctccccatcaaacttgccttcccacagacatgctgctctggctggagcaatgacttggccatctatccaagcatatggcacagagcagctgccagggcagggtgggatggaggtgggggcagagcccagaaggagcctgcctgatgtctcccagttgtggagggcagtctcaccatgaggaatgttggaggtgggtcactgcaggtaccctggtgtgcacgtccgtggagctggttgaaagtggaggcccttggaggccgggcaccattgcttcaagactctggaaggaaccctcccccaggaattcccttgccaccccatgcaggtttctgtacctgcgatgtcaccttctccctatgcacacctgggtgggctttgcctcttctccttaggagagacgtcgggcagagcgggccgagcagcagcgcatccggaatgagcgggagaaggagcggcagaaccgcctggctgtgagtgacccctagcccaggcccatggaggccccccatgctcccagctcctgctgggcccccaggcccctctctattcatcaacatgaatgaaatcagtcaagagacaatattggccgggtgcggtggctcacgcctgtaatcccagcactttgggaggccaaggcgggtagatcacttgaggttgggagttcgagaccatcctgaccaacggagaaaccctgtctctactaaaaatacaaaattagccaggcgtggtggcacatgccggtcatcctagctactcaggaggctgaggcaggagaatcgcttgaacccaggaggcaaggttgcagtgagccgaggtcatgccattgcactctagcctgggcaacaagagtgaaactccaactcaaaaaaaaaaaaaaaggaaaaaaaagagacaatatcaatatcaacctaggaagtgctgggcattgtcttggctctgataatacatcagcaaacaagacagaaatgcctgcgtccttatgaacgtgttattttataagactctccagccctcatcccttccccagtgcagacttcgtgggaaccgaaggccccgtagctctcttcctgtgcagctgctgaaggcatttccagagcaacaggtcttccaggggcactcccagcttaaggggatgacgagacctcacatttgggaatcaggacctcattcaatcacgcagtcatttctcagtcagtggactcctgtgggccagcactgtgctgggagctaccctctcagaaagctccttgctgagcggagagaaagctgaactcacccataaagaccacaagcttcagcccagaatcagggtttccaatcctttcccctaatttgctttcttcctccctgctgtaaatcaggaagagagggctcgacgagaggaggaggagaacaggaggaaggctgaggatgaggcccggaagaagaaggctttgtccaacatgatgcattttgggggttacatccagaaggtaggtagggagcagcaggggttgccaggagatcctagtatagccctgaggaatgaggtgtccactgcagcaggtagactttaggtcaggtcccaggaagagattcccagctgctgtgggtggaagactgaggtctcctcatgcccagccctgagctggttttcagcctgaccgcggcttgcactaggaggggcttgctggggtagggggctacaggcagagcctggggcaccaatggtgccccccatgcctggctccagcagtgctcaggagcccccaaggaggctgggtgcagctggaagagccttgtgcctagagtcagagctctagttggagacccagctctgccacatacagttgtgtgaccttttgcaacctctgagcctcggtttcctcaactggcactcatattgcctgccttgatgctttgagaagctcaaatgagctaccatggaaagtgtctatgtgccacacagatgtgaggttgtttgttcctggaaactcactctcgatccctaacttttgtgccccattcctcctaggtcctccattctcgagggaggagggatatgtgaaattgattttctctaggtagtgaaggaatcaaaacattttcttagatttccagcttagtccctagctcaggactaggccaccccctgcctctgcccccatggtgaccacaccagccaagactttggcatcaaagtcttcatgctaccgaacagcccctgctgtaaccctcagacccacgtcctagtaaacccggctgactacagcgagtatctgaatcttctcttccatgtctctccttgcctttgtcttcctgccttctctctgtgtctctgttgccgtcttggtctttttctatgggcctctctctcatggtttcccggtttagcaggcccaggtgggtaccaaaaacctcaagccttcttcctccccatagttttggagagctccctgggaggggaaggcgccttgctggaggggaggagtgaagagattgggctggctggggaggggggaagctggtgagaggtgaaagagattagttaggcaaagaaggaagagactgacgtgatccttcttgcccctacctcagtttccctagggaatacatgtagttggtagttggggggtgtggcaggaagaagagcataagaacctggccctcctgcaggccttgctccacccccagggggtttggggagggttaggggacagggagggggcaatctggccagtttactctgcttcccacaccctccagacagagcggaaaagtgggaagaggcagactgagcgggaaaagaagaagaagattctggctgagaggaggaaggtgctggccattgaccacctgaatgaagatcagctgaggtgggggctgggaggaatggtcctggggcactgcccgcatctgctctgggaggggtcgggccaagggcacttcttgctgtgagccaccagagggcaggactgcctcacatattttcccatggcaagtttggtgggaccagagctgggccccagcaggtcgggccaactcctttctgggctttgctgatggggaaagagaactgagcaggtgccccacccctctgagcttcacctacccacctgcacacgggtgtcaggacaggggattgaactagatgctctcctgcgccagaagggcttctcctgctccccgcttgttcttggaactttgccctggcagtccctggtgtccgttcctgagcactgggtgctgccgtctggtccccaggctgggtcaggcccagggggtgggctggggtacccttggcaggcctggaggtgggaaggagggctgggatgctgggggctgggccgggaccaggacggaggcccaggagggccctttcttactggacctccccattctcacccttgcccgtgcagggagaaggccaaggagctgtggcagagcatctataacttggaggcagagaagttcgacctgcaggagaagttcaagcagcagaaatatgaggtgggccgccatgctgtccccgcccagcatctccatctcacactcctcctggttcactgggtccggtaatactgctgcaggtccctgggtccctgtccctattccccaacagcccccttcagctcctgcatctgcccctgctgcctggccttcagcagtgttccatgtccaccgcattgacgactgcttgctggaagtgtctgagagctcgctggggctgagcagagacactttcctggtgttccaaccctgggggtctccaacacgttgaggcagcagctcagtgatctgagctggttacaaggaccaggatgcaccaagccaggacccccagtggaaggggagtgctgccaacagagaggtgcttctccccacatacacccaaggtcctggtgtgggcacaattaggctgagcctcaagctcacagtctttcggagtcctatgtgcactaatgagggtcttaggtgaacagacaccaggcaaggaaatggcttagaggacactgatgctgcataccggagcttagacctgggcgccagtccttccttacccaccacccccgagccccggtcccagggcctctttgctacctaagggaagaattcagcttcccctggaaggtctcctttgctgctcctgccaaaccactcctccctgggcaagaagcccctcgctgggcaggctcaggctgcagtggctacagggtactcccacctcccaatacggagagaggctgtattgcctggtgacagtggcatggactttggagccataatgcctgggttgaatttctacctgtgcccctcactggctgtgtgacattgggtgagttagtccactgttccttgcctccatttccacaggtaacactacaatatacttcagagggtgattgtgagggttacagagataatactaattgttattattgctatagtgttccaaccactgttccaagcatgtcccatgtattaacttactatgccctcatagcagccctatgggttcatatctgggaaggtgctcaggacagagcctggcacccactaaatgctcagcaggtgtcagccattgttatggcctctctagtcctgtgccttccacttttttctctttttttggttccacactgaactctgcaccggccaacaggacacagatttgccaaactttggggcagcaccctgcaggggtggtgcatggggatgctactgctcaaagggcacagcttccgggatggtgggcagctgggcaggggtgccccagaggggtctggggctgggctgctaggagggctccatgacacagcctccagctttgtgcccagctctcagaggcccttcttatgggactctcatatcctgaacctattatggccctgggaccccacagtgggaggcccatgaggcatcctggaagcttctccttggcttctgcctgtggtacacgggcccctcctgacccttaactatcctaacccctcctactcttccatgctcctccttctcctcctgcactgctgcactcagcccccttctccccatccccaggccacctgggacctgagccagtcagctccagcgttgctctttgtccttcccacttttcttgcagatcaatgttctccgaaacaggatcaacgataaccagaaagtgtaagtgtctgaggtcattctcgcctagccctccccctgccccctccttccctaccttccagctatcccattcctccttggagggccccaggctcccagcccttcctcctctgcttcagcccacaggtttccttctctggctcctccggggcctcgtgcttctctccctgtggcctctgcttgctggtgggggtgaaatgtggggcggagaagcttccctccagcctcaggctgctgtcctattctgcccctggaggcagctccagcctgctcctctcccctttggcaccccagtcctaccccagccgcatggtgacctactaccctgcctgtgtctccatgtcactgcgtcctgcttcccctgcagctccaagacccgcgggaaggctaaagtcaccgggcgctggaaatagagcctggcctccttcaccaaagatctgctcctcgctcgcacctgcctccggcctgcactcccccagttcccgggccctcctgggcaccccaggcagctcctgtttggaaatggggagctggcctaggtgggagccaccactcctgcctgcccccacacccactccacaccagtaataaaaagccaccacacactgactggcatttctgtcacgtccccttcagagagagagagaaactgaggcgagaccctgctctcacaggtccccggtgtctgccaagctctaaggagctttcccctgtctggtctccctgcttgcctctagctggggagagggtggagggcccagggtgcagcccaggaggcaggagcagtggaaagggcttcagatcattgctcctgggagtcaccaaaacagcacatgggaaaagtgctgttgagggggacagagtgttccctgcatgagtcactcactggagaacaatgcaggactcttcggatggttcccgttctctttctcctcctagccagggccctaggaaccaggtgtctgagggacagggagctcttgaggaaggaaggaaggccagttttattgggcagctattcaggcctgagttgttgagaggtgtgggttagtctgggtgcctcagtttccccctcccatgatctgcacatcctcgagggcttaagtcacagggtcctgagctgaagaggacagcttggtggcatggatttcagttggcagaaagacaaggaaacgatgagagggcaccatcctcctctccagaggagagcctgagacgacacattctcttgaccctccattcctttgtcaggcgtggaatttcccccaattcctgattccttcccttggctccccacttgccatccatcagcaagtgcctcatgagctgtgtctggaaaggcttcctgtggtctcccagccagagcaaatccatttctgcttgctagagcatgtgctgtcacagctatgccatccaatgccacactctgttagacgggtctcttcctgcaggcgtggctctcatgctcccaggagacaatgcagacatgttatcctgtgctgtatgccaagcactgggatccatgtagtgagagatgaagaggaaatggtccttgggccacagggaggggcttagcctggggtctcggcctcatttcttattcattcattcgttcagtcattcaaatacctcctgagcaccaactatgtgctagacactgtgttgggtgtggggtgctgtgatttaagccagcacatcgctcatggagctcacagagctcacatccagcagcagggagacagcaagcagaggtcccagccctgggtgacaggggcgggtggggggtgcgatgctggggagaggcatagccacgcaccacacctggccctggcaggtgggaaggctttctggaggaaagagtgtctaggccgagtaccagagggtgaggagggggaagtcaaggggaagaggttgggggtggggctgggaacggcacatagaaagtttgagaagcaagagccagcatggtgtgtttgaagagatatagttattagcagaatccaagcatcttctggtggggacaagaaggctccatcataaatgcccccaaaatggacagggaggcaggacgcaggcctcaggcccaaggagggctgtcaccacgggctctgggagacccactggcagctcctggcacgcaccagccctactatctcagtttgccggataggacagctggggcagtgccctagcccctctccccataaaggggcactgcacctctacccccacctgccgccttggctccaccgcctatgcctcagtgtctccttggtgcaacaagaggtcatgatgggccgtgtgtctggggccctgtctcctgctccccctctcccagcagggtccttgtgaccatttctctagtacagcggtccccaacctttttggcaccaaggaccagtttcttggaagactatttttccatggacaggggtggggtggggaagaaaagtaggggtgggtgggtgggggtggtttcaggatgaaactattccacttcagatcgtcaggcaatagattctcgtaaggagtatgcaccctagatccctcgcatgcacagttcacaataggtttcacactcctatgagaatctaatgctgctggctgct

SEQ ID NO: 9 - DNA, 5832 bp, Homo sapiens
  source 1..5832: mol_type = genomic DNA
actcgcaggggaggcggagaaggaggcggagggagcgattgtggccccggccgcggtggccggcgcggcctgccctttgtgaccgcagctcgcgccccacgccccgcgcccatggccgccgtgccgggctccctggccacgcgtgcccgcccgcggacctgagccccgcgcctgggatgccggggatgcgcgtcccccggccctgcggctgctccgggctgggcgcggggcgatggacctgagcatgaagaagttcgccgtgcgcaggttcttctctgtgtacctgcgcaggaagtcgcgctccaagagctccagcctgagccggctcgaggaagaaggcgtcgtgaaggagatagacatcagccatcatgtgaaggagggctttgagaaggcagatccttcccagtttgagctgctgaaggttttaggacaaggatcctatggaaaggtgttcctggtgaggaaggtgaaggggtccgacgctgggcagctctacgccatgaaggtccttaagaaagccaccctaaaagttcgggaccgagtgagatcgaagatggagagagacatcttggcagaagtgaatcaccccttcattgtgaagcttcattatgcctttcagacggaaggaaagctctacctgatcctggacttcctgcggggaggggacctcttcacccggctctccaaagaggtcatgttcacggaggaggatgtcaagttctacctggctgagctggccttggctttagaccatctccacagcctggggatcatctacagagatctgaagcctgagaacatcctcctggatgaagaggggcacattaagatcacagatttcggcctgagtaaggaggccattgaccacgacaagagagcgtactccttctgcgggacgatcgagtacatggcgcccgaggtggtgaaccggcgaggacacacgcagagtgccgactggtggtccttcggcgtgctcatgtttgagatgctcacggggtccctgccgttccaggggaaggacaggaaggagaccatggctctcatcctcaaagccaagctggggatgccgcagttcctcagtggggaggcacagagtttgctgcgagctctcttcaaacggaacccctgcaaccggctgggtgctggcattgacggagtggaggaaattaagcgccatcccttctttgtgaccatagactggaacacgctgtaccggaaggagatcaagccaccgttcaaaccagcagtgggcaggcctgaggacaccttccactttgaccccgagttcacagcgcggacgcccacagactctcctggcgtccccccgagtgcaaacgctcatcacctgtttagaggattcagctttgtggcctcaagcctgatccaggagccctcacagcaagatctgcacaaagtcccagttcacccaatcgtgcagcagttacacgggaacaacatccacttcaccgatggctacgagatcaaggaggacatcggggtgggctcctactcagtgtgcaagcgatgtgtgcataaagccacagacaccgagtatgccgtgaagatcattgataagagcaagagagacccctcggaagagattgagatcctcctgcggtacggccagcacccgaacatcatcaccctcaaggatgtctatgatgatggcaagtttgtgtacctggtaatggagctgatgcgtggtggggagctcctggaccgcatcctccggcagagatacttctcggagcgcgaagccagtgacgtcctgtgcaccatcaccaagaccatggactacctccattcccagggggttgttcatcgagacctgaagccgagtaacatcctgtacagggatgagtcggggagcccagaatccatccgagtctgcgacttcggctttgccaagcagctgcgcgcggggaacgggctgctcatgacaccctgctacacggccaatttcgtggccccggaggtcctgaagcgtcaaggctatgatgcggcgtgtgacatctggagtttggggatcctgttgtacaccatgctggcaggatttaccccttttgcaaatgggccagacgatacccctgaggagattctggcgcggatcggcagtgggaagtatgccctttctgggggaaactgggactcgatatctgacgcagctaaagacgtcgtgtccaagatgctccacgtggaccctcatcagcgcctgacggcgatgcaagtgctcaaacacccgtgggtggtcaacagagagtacctgtccccaaaccagctcagccgacaggacgtgcacctggtgaagggcgcgatggccgccacctactttgctctaaacagaacacctcaggccccgcggctggagcccgtgctgtcatccaacctggctcagcgcagaggcatgaagagactcacgtccacgcggctgtagcgggtgggaccctggccccagcgtcccctgccagcatcctcgtgggctcacagaccccggcctcggagcccgtctggcacccagagtgaccacaagtccagcagggaggcggcgcccgccctcgccgtgtccgtgttttctttttcagccccggagagggtcctgacctgggggcttctccaagcctcactgcgccagcctccccgcccgctctcttttctcccaagcgaaaccaaatgcgccccttcacctcgcgtgcccgtgcgaggccgggggcttctttcagagcccgcgggtcctctcatacatggcttctgtttctgccgagagatctgttttccaattatgaagccggtcggtttggtcagactcccgacacccacgtcccaggtacccggtgggaaagtggcagtgcgagggcgcagccattggtggttgcagggccccagagggctggggtgacctggcatcccggggctccccacgggctggatgacggggttggcactgtggcgtccaggaggagatgcctggttctgcccaaaataatccaaagagccgtttcctcctcgcccttcagtttttgcctgaggtgctgggtagcccatcctttcctctgtcccagattcaaatgaggagtaagagcccagacgagaggaaggcaggctggatctttgccttgagagctccgtgtcaccaggatggaagggggtgcctctcggaggagcctgtgtccacctccagtctcggctttccccggggggccaagcgcactgggctgccgtctgtccccagctcccgtggccacacagctatctggaggctttgcagggagtcgtgggttctcgcacctgctcagccctgtgtcggcttcctgtgtgctcacctaaagctgtggttttgctgtgttcacttcgatttttctggtctgtggagaaactgtgaattggagaaatggagctctgtggcttcccacccaaaccttctcagtccagctggaggctggagggagacacaggccccacccagcagactgaggggcagaggcacaggtgggagggcagcggagatcagcgtggacaggagcgatgcactttgtagatgctgtggctttgtgttgcgttttgtgtctctgttgcacagatctgttttttcacactgatccgtattcccctgggtgtgcacacagggcgggtgtggggcatttaggccatgctgtgctctacttcattgagtaaaatcgagtgagaggttccgggcagcaggatcgacgcccagtccagccggcagagggaacacacgggtccttcattgtcctgtaagggtgttgaagatgctccctggcggcccccaagcagactagatgggaggaggcgccgctcagcccctcaccctgcatcactgaagagcggcgcctctgcagcaagcagggcttcaggaggtgcccgctggccacagccaggttttccctaagaagatgttattttgttgggttttgttccccctccatctcgattctcgtacccaactaaaaaaaaaaaaataaagaaaaaatgtgctgcgttctgaaaaataactccttagcttggtctgattgttttcagaccttaaaatataaacttgtttcacaagctttaatccatgtggattttttttttcttagagaaccacaaaacataaaaggagcaagtcggactgaatacctgtttccatagtgcccacagggtattcctcacattttctccatagaagatgctttttcccaaggctagaacgacttccaccatgatgaatttgctttttaggtcttaattatttcacttctttttagaaacttaggaagaagtggataatcctgaggtcacacaatctgtcctcccagaaatgaacaaaagtcatcaccttttctgcttgctacacaggcaacgattcccccatcagctgcccggaccctttggcctggcttggtgtgcaggcctgtctgtttgcttaaagtcagtgggttctggtgcagggagtgagaagtgggggaagtgaaagggaaagcatccgtgagaaagcggccacggttttccctccttgtgtgcccatggggcaccagctcatggtctttttcagtcatcccagtttgtacagacttagcttctgaactctaagaatgccaaagggaccgacgagactccccatcacagcgagctctgtccttacatgtatttgatgtgcatcagcggaggagaacactggcttggccctgctccgctgagtgtctgtgaaatacctctactttccctcccatatccagaacaaaatgatacttgacatccttccacaaaagtcagcctaaagaagttatggtatcatatgttaaactaagctttcaaaaaccttagtgaaatagcaagtgactgctttcaagcagcagtcgacatgtaaatgaaggtgttcttagaattcgcattttgccagctcagcgcacctccacaacgaatgaaatgctccgtatgatttgcacaaatgacatagacctccccaaaagttaactggctctccttcctcacacagttcatcataacccaaccccccacccccgggtcatgaaaatcacagaacttataaacacattgaaccctagatctcaggcttcctgacctaccgccagtggccccttgctggccaccctatagggtcctccttccctggcagccccccatgtgggagaaatacctgattctcccaatctgcagtgggagagctttgctgaattccatcccaaagtcaaacatgggcaagaggtgaggatttcacttttaccctcaagtccgatttgtctgtgattttaaactaactgtgtatgtattgatgtttggaagattgtttgaattttaaagtgataatagtacttaatgttatccagtattgttcattaaatggtgttatcctaaagctgcacttgggatttttacctaacgctttactgattctctcaagcacatggcaaagtttgatttgcactccgttcatttctgacacgttttgctgcctcctacctttctaagcgtcatgcaaattcgagaatggagaaggacgctgccggtccctgagcggtgtggagagggcggaaggtggactccagcgcagcttgaggggctgaggacggaggctgcagcatctgtgtcgttctactgagcacgcttctctgcctcgctcctgactcagcactttgttcactggctcagcagttatgtttacacatcatttttatgttcctgctttgtaattcatgtttgagatgggtggccactgtacagatatttattacgctttccagactttctgaatagatttttttgaataaacatggttttatgaagtgtaatctttttctagcctaacaataa

SEQ ID NO: 10 - protein, 733 aa, Homo sapiens
MDLSMKKFAVRRFFSVYLRRKSRSKSSSLSRLEEEGVVKEIDISHHVKEGFEKADPSQFELLKVLGQGSYGKVFLVRKVKGSDAGQLYAMKVLKKATLKVRDRVRSKMERDILAEVNHPFIVKLHYAFQTEGKLYLILDFLRGGDLFTRLSKEVMFTEEDVKFYLAELALALDHLHSLGIIYRDLKPENILLDEEGHIKITDFGLSKEAIDHDKRAYSFCGTIEYMAPEVVNRRGHTQSADWWSFGVLMFEMLTGSLPFQGKDRKETMALILKAKLGMPQFLSGEAQSLLRALFKRNPCNRLGAGIDGVEEIKRHPFFVTIDWNTLYRKEIKPPFKPAVGRPEDTFHFDPEFTARTPTDSPGVPPSANAHHLFRGFSFVASSLIQEPSQQDLHKVPVHPIVQQLHGNNIHFTDGYEIKEDIGVGSYSVCKRCVHKATDTEYAVKIIDKSKRDPSEEIEILLRYGQHPNIITLKDVYDDGKFVYLVMELMRGGELLDRILRQRYFSEREASDVLCTITKTMDYLHSQGVVHRDLKPSNILYRDESGSPESIRVCDFGFAKQLRAGNGLLMTPCYTANFVAPEVLKRQGYDAACDIWSLGILLYTMLAGFTPFANGPDDTPEEILARIGSGKYALSGGNWDSISDAAKDVVSKMLHVDPHQRLTAMQVLKHPWVVNREYLSPNQLSRQDVHLVKGAMAATYFALNRTPQAPRLEPVLSSNLAQRRGMKRLTSTRL

SEQ ID NO: 11 - DNA, 10343 bp, Homo sapiens
  source 1..10343: mol_type = genomic DNA
catcatgcagcaggtcaaacaaggcatctcctagtattgcatcctacagatgtgctgtaaacatcaaaagaagacggtgggatcaggagatgctgttttggaaagaagtgaggtttagacttctccatgttaaccatgagcgtgacactttcccccctgaggtcacaggacctggatcccatggctactgatgcttcacccatggccatcaacatgacacccactgtggagcagggtgagggagaagaggcaatgaaggacatggactctgaccagcagtatgaaaagccacccccactacacacaggggctgactggaagattgtcctccacttacctgaaattgagacctggctccggatgacctcagagagggtccgagacctaacctattcagtccagcaggattcggacagcaagcatgtggatgtacatctagttcaactaaaggacatttgtgaagatatttctgatcatgttgagcaaatccatgccctccttgaaacagagttctccctaaagctgctgtcttactctgtcaacgtgatagtggacatccacgcagtgcagctcctctggcaccagcttcgagtctcagtgctggttctgcgggagcgcattctgcaaggtctgcaggacgccaatggcaactacactaggcagacggacattctgcaagctttctctgaagagacaaaagagggccggcttgattctctaacagaagtggatgactcaggacaattaaccatcaaatgttctcaaaattacttgtctctggattgtggcattactgcattcgaactgtctgactacagtccaagtgaggatttgctcagtgggctaggtgacatgacctctagccaagtcaaaaccaaaccctttgactcttggagctacagtgagatggaaaaggagtttcctgagcttatccgaagtgttggtttacttacggtagctgctgactctatctctaccaatggcagtgaagcagttactgaggaggtatctcaagtatctctctcagtagacgacaaaggtggatgtgaggaagacaatgcttctgcagtcgaagagcaaccaggcttaacactgggggtgtcatcatcttcaggagaagctctgacaaatgctgctcaaccctcctctgagactgtgcagcaagaatccagttcctcctcccatcatgatgcaaagaatcagcagcctgttccttgtgaaaatgcaacccccaaacgaaccatcagagattgctttaattataacgaggactctcccacgcagcctacattgccaaaaagaggactttttcttaaagaggaaacttttaagaatgatctgaaaggcaatggtggaaagaggcaaatggttgatctaaagcctgagatgagcagaagcaccccttcgctagtagatcctcctgacagatccaaactttgcctggtattgcagtcttcttaccccaacagcccttctgctgccagccagtcttatgagtgtttacacaaggtggggaatgggaaccttgaaaacacagtcaaatttcacattaaagaaatttcttccagcctgggaaggcttaacgactgctataaagagaaatctcgacttaaaaagccacacaagacctcagaagaggtgcctccatgccgaacacctaaacgggggactggttcaggcaaacaagctaaaaatacaaagagctcagcagtgccaaatggagagctttcttatacttccaaggccatagaggggccacaaacaaattctgcttccacatcctcacttgagccttgtaatcagagaagttggaatgccaaattgcaattgcagtcagaaacatccagttcaccagcttttactcagagcagtgaatcctctgttggctcagacaacatcatgtctccggtgccacttctttcaaaacacaaaagcaaaaaaggtcaagcctcctctccaagtcacgtcactaggaatggtgaggttgtggaggcctggtatggctctgatgaatacctagcactgccctctcaccttaagcagacagaagtattggctttgaagttggaaaacctaacaaagcttctgcctcagaaacccagaggagaaaccatccagaatattgatgactgggaactgtctgaaatgaattcagattctgaaatctatccaacctatcatgtcaaaaagaagcatacaaggctaggcagggtgtctccaagctcatctagtgacatagcctcttcactaggggagagcattgaatctgggcccctgagtgacattctttctgatgaggagtccagtatgcctctcgctggcatgaaaaagtatgctgatgagaagtcagaaagagcttcatcctctgagaaaaatgagagccattctgccactaaatcagctttaattcagaaactgatgcaagatattcagcaccaagacaactatgaagccatatgggaaaaaatagaggggtttgtaaacaaactggatgaattcattcaatggttaaatgaagccatggaaactacagagaattggactccccctaaagcagagatggatgaccttaaactgtatctggagacacacttgagttttaagttgaatgtagacagtcattgtgctctcaaggaagctgtggaggaggaaggacaccaacttcttgagcttattgcatctcacaaagcaggactgaaggacatgctgcggatgattgcaagtcaatggaaggagctgcagaggcaaatcaaacggcagcacagctggattctcagggctctggataccatcaaagccgagatactggctactgatgtgtctgtggaggatgaggaagggactggaagccccaaggctgaggttcaactatgctacctggaagcacaaagagatgctgttgagcagatgtccctcaagctgtacagcgagcagtataccagcagcagcaagcgaaaggaagagtttgctgatatgtcaaaagttcattcagtgggaagcaatgggcttctggactttgattcagaatatcaggagctctgggattgcttgattgacatggagtcccttgtgatggacagccacgacctgatgatgtcagaggagcagcagcagcatctttacaagcgatacagtgtggaaatgtccatcagacacctgaaaaagacggagctgcttagtaaggttgaagctttgaagaaaggtggcgttttactaccaaatgatctccttgaaaaagtggattcaattaatgaaaaatgggaactgcttgggaaaaccctaggagagaagatccaggacacaatggcagggcacagtgggtcgagtccacgtgacctgctctctcctgaaagtggaagcctggtaaggcagctggaggtcaggatcaaagaactgaaaggatggctaagagatacagagcttttcatcttcaattcctgtctgagacaagaaaaggaaggaacaatgaatactgagaaacaactgcaatactttaagtccctctgtcgtgaaatcaagcaacgacgtcgaggagttgcctccattctgcgactatgccagcatcttttggatgaccgggagacttgcaatctgaatgcagaccaccagcccatgcagctgatcattgtaaatcttgaaagaaggtgggaagccattgtcatgcaagccgtccagtggcaaacacgtctacaaaagaagatgggaaaggaatctgagactttgaatgtgattgatcctggcttgatggacctaaatgggatgagtgaggatgccctggaatgggatgaaatggacataagtaacaagttaattagtttgaatgaggaatcaaatgaccttgatcaagaactccaacctgttatcccttccttgaagcttggagagacaagtaatgaggaccctggttatgacgaggaggctgataaccatgggggatctcagtatgcctcaaatattactgccccctctagtccacacatttaccaggtgtacagcctccacaatgttgaactctatgaggacaaccacatgccatttctgaaaaacaatccaaaggtcactggcatgacacagcctaatgttttaactaagagtctcagtaaagactcttcattttcatctaccaaatctttgccagatcttctaggtggttccaatttggtaaagccctgcgcatgtcatggaggagacatgagccagaattcaggcagtgagagtggaattgtcagtgaaggagacacagaaaccactaccaactctgaaatgtgcttgctcaatgcagtggatgggtccccaagtaaccttgaaactgaacatctggacccacaaatgggagatgcagttaacgtgttaaagcaaaaatttacagatgagggggaaagcattaagcttccaaatagctctcagtcgtccatttcaccagtgggttgtgtaaatggaaaagttggagatttaaacagtattaccaaacatacccctgactgtttgggagaagaattacaaggaaaacatgatgtgtttacattttatgattactcatacctccaaggctcaaaactcaaattaccaatgataatgaaacagtcacaaagcgaaaaagtgcatgtggaggatcccctgcttcgtggtttttattttgataaaaaatcatgcaaatctaaacatcagactacagagttacaaccagatgtacctccccatgaaaggattttggcaagtgcatctcatgaaatggatcgcatttcatataaaagtggcaatatagaaaagacattcactggcatgcagaatgccaaacagctctcccttttatctcatagttcatctattgagtccctttctccagggggtgatttatttggattgggcatctttaaaaatggcagtgacagcctccagcgaagcacttctttagaaagttggttgacttcctataaaagcaatgaagatctctttagctgtcacagctctggggatataagcgtgagcagtggctcagttggtgaactaagtaaaagaacattagatctcctgaatcgtttggagaatatccagagcccctcagagcaaaagataaaacgaagtgtttctgatatcactcttcaaagcagttcccaaaagatgtcctttactggccagatgtcattggacatagcatcttctatcaatgaagactcagcggcatctctaacagaacttagcagcagtgacgagctctctctttgctcagaggatattgtgttacacaagaacaagatcccggaatcgaatgcatcgttcaggaagcgtctgactcgttcagtggctgatgaaagcgatgtcaatgtcagcatgattgttaatgtctcttgcacctctgcttgcactgatgatgaagatgacagcgacctgctctccagctctacccttaccttgactgaagaagagctgtgcatcaaagatgaggatgacgactccagtattgcaacagatgatgaaatttatgaagactgcaccttgatgtcagggctagactacataaagaatgaattacagacctggattaggccaaaattgtctttgacaagagataagaaaaggtgcaatgtcagtgatgagatgaagggcagtaaagatataagtagcagtgagatgaccaatccctctgatactctgaatattgagacccttctaaatggctctgtaaaacgtgtctctgaaaataatggaaatggtaagaattcatctcatacccatgagttagggacaaagcgtgaaaataagaaaactattttcaaagttaataaagatccatatgtggctgacatggaaaatggcaatattgaaggtattccagaaaggcaaaagggcaaaccgaatgtgacttcaaaggtatcagaaaatcttggttcacatgggaaagagatttcagagagtgagcattgtaagtgtaaagcacttatggatagtttagatgattcaaatactgctggcaaggaatttgtttcccaagatgttagacatcttccaaagaaatgtccaaatcaccaccattttgaaaatcaaagcactgcctctactcccactgagaagtctttctcagaactggctttagaaaccaggtttaacaacagacaagactctgatgcactgaaatcatctgatgatgcaccgagtatggctggaaaatctgctggttgttgcctagcacttgaacaaaacggaacagaggaaaatgcttctatcagcaacatttcctgttgcaactgtgagccagatgttttccatcaaaaagatgccgaagattgttcagtacacaactttgttaaggaaatcattgacatggcttcgacagccctaaaaagtaaatctcaacctgaaaacgaggtggctgctcctacttcattaactcaaatcaaggagaaagtgttggagcattctcaccggcccatccagctgagaaaaggggacttttattcgtacttatctctctcatctcatgacagtgattgtggggaggtcaccaattacatagaagagaaaagcagcactccattgccactagacaccactgactcgggcttagatgacaaggaagatattgaatgcttttttgaggcctgtgttgagggtgactctgatggagaggagccttgtttctctagtgctcctccaaatgaatctgcagttcccagcgaagctgcaatgccactacaagcaacagcatgttcttctgagttcagtgatagttctctttcagctgatgatgcagatacagtggctctttcaagtccttcctctcaggaaagagctgaggttggaaaggaagtgaatggtttgccccaaacttccagtggctgtgcagaaaacttagagtttactccttcaaagcttgacagtgaaaaggaaagttccggaaaaccaggtgaatctggaatgccagaagaacataatgctgcttcagccaaatctaaagttcaagacctctccttgaaggcaaatcagccaacagacaaggccgcattgcatcccagccccaaaactttaacctgtgaagaaaatcttctaaaccttcatgaaaaacgacatagaaatatgcataggtagaatgtaccccctccccaagcatgaaaatcatctcactgaaagatacgcctggctgcaactcaggggtggcctcatcctcccgccctgggctggcctctggttccatcacgtttgtcactgccgtttattacattgacttctcccaagatgaatcttccttccaaatgtgttttctccacacaagccttgtgatctgaatgtgtgcgctggttctctttaggtgatcgtctttgaagttcagcaaagctgcttgttctcccatggattcctgtcccaagctacctctaccaaccctctctctccagctagacttttctctttgcctcctcccttcccttccactctttaaagttctgcagttcaccaactggtagtccattaaattctcctgtctagaatgacccccccaccagtacttgaccaatttcatgtatcaatctggattttttttttaacggtataatgactgtgcttattgaaagagttttacctaaaaagccaacatttgaattggttgcagcatagagaagaaacactggtccttctttcaaaattaagcaactattaaaagcgccattttatttatttcatttaaaaaataatctatgcagcatttcaagaaacaaccatatggtgttgtatattataaactggtgacattctactattgaattatgtacaacattttcattttttatgcttcttgaggtggtaatgagaaaaaagttttttaaaaaagtgtgccttgctgtatttcttataccatttattaaaaagctgctttcacggtaaaattatgttggtttgaaaggaggaaatagcaaggttaagatgtgtgaataatttctgtatatatgtataaccaagtacaaacattgatgtataatgacagtataaaatgctttcatgtttgtgatgtctagtgatgtggaaaatataagccttaaatccattagattgcatggtaattaaaattggcataataaacacagattattgggggaaaaggaaaattagtgatctcttctactatgttctttaccaaattgttgcatctggttctgaaaaagtatagcatgtagcagcttccaaacatattcatattgcttaagaggcttaacattacctaaactagagactagacgtaaagccttcagttttcaaaatctttctggtcactataaagatcttggaacagcaaatgattaaatgtcagttcccctaaaccaataaacatttatactagattttttatttccacttatcattaatgatttaatgttggatttcaggtaccttgtatgtcttaatttattttaaatatttattttgaatgagtttgatagaaagctagtagaaaagtacagaaaatttgactattatttatagatttcaggtatatttatatgtgtaaaagaaattgacaaagaaatatttcatctggcctttactgactcctgttaaatgcagttttaaatttatatcgtaacacctacttaagtgcctgacacagtaggtattcaataaaaatttactgaattaaaggattaaattaggtgacatggtgacatctatccctttattttgacactaaaacatggacacaactagaaagaggtacaatgcaatataaagtcacaatagataatatatatcaaatttctaaaaggtaaagaatgttgtgggttcatgcagtcacaggaatgacaatcattcaacagatagttcagaaacactttttatctgcaaggcactattctagatccagaagatgcaatgttgaacaaacagacaaagccctgccctcagaaggctgtcctgcattaggaacaagtgaacacgcaaatgacatgaagtatttgttgcagagctgaggaacagagcaaatgtagtgatagaagcgcaatgagagaagcagcagtgggtacaaggaggaagaaaaagggcttgcagagagtggaaagttagtggaatattcatgaaacttcattgcaggggtaatagaagaaaaagtaaattgggaggacttaatggaaggtcttttaaaaagttaacttggagcttctgtatgtaaaatgctaggtaataaggacactttgtacaggctgttttgcacctgattttatttatcattagtgccacgccaagatcatttagacgatgcttatctgtaattctaccactttaataactatttgtatttttatgccccttctgatcttttccatatgtatttctaaatggataaattattctaggcttcttaataggtagtaatttgttcaaaagcggttttagccagacatctagttgcagtgttcaagaggattatgggggaaagagattagagataattgtctagttagggggcagctggagaaaataagctaagtttgcaataacagagtacacaagtatagtggcccaggatgtagtgaaagaacaaatcctagagtctttgaaatttctaagggcattctagacctctgttgggatatggtattattttacatactgacacaacctaaattttctttgggtagtaactaatgtcaagtctacatcgactggtaaaacattcaaagaacaaactgacaatgatgttctacctacttgttacatgctcatggaagaccgtgcagtattgaaagtatttgttaattatctgcttagtattaacactaaatttgtagaatgactttcaggtttgttgaacaatgccttttcaggttggaagaagaaaaatagcctcaatctcccaccccatgtaggcactacctccccaattacccttagaaaatgatcacaccaactctgcctacacacttccagtgatagtggctcattgtctgttaaggcaaactgttccactgttgggcatatctctttgttagaaagttctttcttaggttgctaaaatctgcctagtaccccgctaccctgttctgtcttatggagcagcccagattatctttactccctctttctcatggcaaccctgaagataatcaaggccagttactcatcatctcccaaccactgtttcctcaactgcccttcatatgtcatggttttcagatccattccaacctgactgaatgttaacagacagaattcttcacattaaggaactgtcttcatcatcatacatgtagaaaagaatctgaacatttaagtgcgaagttttctctagaaatatattcaagatatgtttattctattattgtaaatttcaaacaataaataaataagaatcc

SEQ ID NO: 12 - protein, 2319 aa, Homo sapiens [sequence omitted]

SEQ ID NO: 13 - protein, 2329 aa, Homo sapiens
MLTMSVTLSPLRSQDLDPMATDASPMAINMTPTVEQGEGEEAMKDMDSDQQYEKPPPLHTGADWKIVLHLPEIETWLRMTSERVRDLTYSVQQDSDSKHVDVHLVQLKDICEDISDHVEQIHALLETEFSLKLLSYSVNVIVDIHAVQLLWHQLRVSVLVLRERILQGLQDANGNYTRQTDILQAFSEETKEGRLDSLTEVDDSGQLTIKCSQNYLSLDCGITAFELSDYSPSEDLLSGLGDMTSSQVKTKPFDSWSYSEMEKEFPELIRSVGLLTVAADSISTNGSEAVTEEVSQVSLSVDDKGGCEEDNASAVEEQPGLTLGVSSSSGEALTNAAQPSSETVQQESSSSSHHDAKNQQPVPCENATPKRTIRDCFNYNEDSPTQPTLPKRGLFLKEETFKNDLKGNGGKRQMVDLKPEMSRSTPSLVDPPDRSKLCLVLQSSYPNSPSAASQSYECLHKVGNGNLENTVKFHIKEISSSLGRLNDCYKEKSRLKKPHKTSEEVPPCRTPKRGTGSGKQAKNTKSSAVPNGELSYTSKAIEGPQTNSASTSSLEPCNQRSWNAKLQLQSETSSSPAFTQSSESSVGSDNIMSPVPLLSKHKSKKGQASSPSHVTRNGEVVEAWYGSDEYLALPSHLKQTEVLALKLENLTKLLPQKPRGETIQNIDDWELSEMNSDSEIYPTYHVKKKHTRLGRVSPSSSSDIASSLGESIESGPLSDILSDEESSMPLAGMKKYADEKSERASSSEKNESHSATKSALIQKLMQDIQHQDNYEAIWEKIEGFVNKLDEFIQWLNEAMETTENWTPPKAEMDDLKLYLETHLSFKLNVDSHCALKEAVEEEGHQLLELIASHKAGLKDMLRMIASQWKELQRQIKRQHSWILRALDTIKAEILATDVSVEDEEGTGSPKAEVQLCYLEAQRDAVEQMSLKLYSEQYTSSSKRKEEFADMSKVHSVGSNGLLDFDSEYQELWDCLIDMESLVMDSHDLMMSEEQQQHLYKRYSVEMSIRHLKKTELLSKVEALKKGGVLLPNDLLEKVDSINEKWELLGKTLGEKIQDTMAGHSGSSPRDLLSPESGSLVRQLEVRIKELKGWLRDTELFIFNSCLRQEKEGTMNTEKQLQYFKSLCREIKQRRRGVASILRLCQHLLDDRETCNLNADHQPMQLIIVNLERRWEAIVMQAVQWQTRLQKKMGKESETLNVIDPGLMDLNGMSEDALEWDEMDISNKLISLNEESNDLDQELQPVIPSLKLGETSNEDPGYDEEADNHGGSQYASNITAPSSPHIYQVYSLHNVELYEDNHMPFLKNNPKVTGMTQPNVLTKSLSKDSSFSSTKSLPDLLGGSNLVKPCACHGGDMSQNSGSESGIVSEGDTETTTNSEMCLLNAVDGSPSNLETEHLDPQMGDAVNVLKQKFTDEGESIKLPNSSQSSISPVGCVNGKVGDLNSITKHTPDCLGEELQGKHDVFTFYDYSYLQGSKLKLPMIMKQSQSEKVHVEDPLLRGFYFDKKSCKSKHQTTELQPDVPPHERILASASHEMDRISYKSGNIEKTFTGMQNAKQLSLLSHSSSIESLSPGGDLFGLGIFKNGSDSLQRSTSLESWLTSYKSNEDLFSCHSSGDISVSSGSVGELSKRTLDLLNRLENIQSPSEQKIKRSVSDITLQSSSQKMSFTGQMSLDIASSINEDSAASLTELSSSDELSLCSEDIVLHKNKIPESNASFRKRLTRSVADESDVNVSMIVNVSCTSACTDDEDDSDLLSSSTLTLTEEELCIKDEDDDSSIATDDEIYEDCTLMSGLDYIKNELQTWIRPKLSLTRDKKRCNVSDEMKGSKDISSSEMTNPSDTLNIETLLNGSVKRVSENNGNGKNSSHTHELGTKRENKKTIFKVNKDPYVADMENGNIEGIPERQKGKPNVTSKVSENLGSHGKEISESEHCKCKALMDSLDDSNTAGKEFVSQDVRHLPKKCPNHHHFENQSTASTPTEKSFSELALETRFNNRQDSDALKSSDDAPSMAGKSAGCCLALEQNGTEENASISNISCCNCEPDVFHQKDAEDCSVHNFVKEIIDMASTALKSKSQPENEVAAPTSLTQIKEKVLEHSHRFIGURECONTPIQLRKGDFYSYLSLSSHDSDCGEVTNYIEEKSSTPLPLDTTDSGLDDKEDIECFFEACVEGDSDGEEPCFSSAPPNESAVPSEAAMPLQATACSSEFSDSSLSADDADTVALSSPSSQERAEVGKEVNGLPQTSSGCAENLEFTPSKLDSEKESSGKPGESGMPEEHNAASAKSKVQDLSLKANQPTDKAALHPSPKTLTCEENLLNLHEKRHRNMHR

SEQ ID NO: 14 - protein, 190 aa, Homo sapiens
MGKSSTPLPLDTTDSGLDDKEDIECFFEACVEGDSDGEEPCFSSAPPNESAVPSEAAMPLQATACSSEFSDSSLSADDADTVALSSPSSQERAEVGKEVNGLPQTSSGCAENLEFTPSKLDSEKESSGKPGESGMPEEHNAASAKSKVQDLSLKANQPTDKAALHPSPKTLTCEENLLNLHEKRHRNMHR

SEQ ID NO: 15 - protein, 142 aa, unidentified
MVELSLCSEDIVLHKNKIPESNASFRKRLTRSVADESDVNVSMIVNVSCTSACTDDEDDSDLLSSSTLTLTEEELCIKDEDDDSSIATDDEIYEDCTLMSGLDYIKNELQTWIRPKLSLTRDKKRCNVSDEMKGSKDISSSE

SEQ ID NO: 16 - protein, 142 aa, Homo sapiens
SDELSLCSEDIVLHKNKIPESNASFRKRLTRSVADESDVNVSMIVNVSCTSACTDDEDDSDLLSSSTLTLTEEELCIKDEDDDSSIATDDEIYEDCTLMSGLDYIKNELQTWIRPKLSLTRDKKRCNVSDEMKGSKDISSSE

SEQ ID NO: 17 - protein, 142 aa, Macaca mulatta
SDELSLCSEDIVLHKNKIPESNASFRKRLTRSVADESDVNVSMIVNVSCTSACTDDEDDSDLLSSSTLTLTEEELCIKDEDDDSSIATDDEIYEDCNLMSGLDYIKNELQTWIRPKLSLTREKKRCNVSDEMKGSKDVSSSE

SEQ ID NO: 18 - protein, 142 aa, Pan paniscus
SDELSLCSEDIVLHKNKIPESNASFRKRLTRSVADESDVNVSMIVNVSCTSACTDDEDDSDLLSSSTLTLTEEELCIKDEDDDSSIATDDEIYEDCNLMSGLDYIKNELQTWIRPKLSLTRDKKRCNVSGEMKGSKDVSSSE

SEQ ID NO: 19 - protein, 142 aa, Loxodonta africana
SDELSLCSEDIVLHKNKIPESNASFRKRLTRSVADESDVNVSMIVNVSCTSACTDDEDDSDLLSSSTLTLTEEELCIKDEDDDSSIATDDEIYEDCNLMSGLDYIKNELQTWIRPKLSLTRDKRRCNLSDEIKSSKDVSSSE

SEQ ID NO: 20 - protein, 142 aa, Choloepus hoffmanni
SDELSLCSEDIVLHKNKIPESNASFRKRLTRSVADESDVNVSMIVNVSCTSACTDDEDDSDLLSSSTLTLTEEELCIKDEDDDSSIATDDEIYEESNLMSGLDYIKNELQTWIRPKLSLTRDKKRCNLSDEIKGSKDVSSGE

SEQ ID NO: 21 - protein, 142 aa, Tursiops truncatus
SDELSLCSEDIVLHKNKIPESNASFRKRLPRSVADESDVNVSMIVNVSCTSACTDDEDDSDLLSSSTLTLTEEELCIKDEDDDSSIATDDEIYEECNLMSGLDYIKNELQTWIRPKFSLTRDKRRCNLSDEIKGSKDVSSSE

SEQ ID NO: 22 - protein, 142 aa, Phyllostomus hastatus
SDELSLCSEDIVLHKNKIPESNASFRKRLTRSVADESDVNVSMIVNVSCTSACTDDEDDSDLLSSSTLTLTEEELCIKDEDDDSSIATDDEIYEDCNLMSGLDYIKNELHTWIRPKLSLVRDKRRCNLSDEIRGSKDISSNE

SEQ ID NO: 23 - protein, 142 aa, Equus asinus
SDELSLCSEDIVLHKNKIPESNASFRKRLTRSVADESDVNVSMIVNVSCTSACTDDEDDSDLLSSSTLTLTEEELCIKDEDDDSSIATDDEIYEECNLMSGLDYIKNELQTWIRPKYSLTRDKRRCNLSDEIKGCKDVSSSE

SEQ ID NO: 24 - protein, 142 aa, Dasypus novemcinctus
SDELSLCSEDIVLHKNKIPESNASFRKRLTRSVADESDVNVSMIVNVSCTSACTDDEDDSDLLSSSTLTLTEEELCIKDEDDDSSIATDDEIYEECNLMSGLDYIKNELQTWIRPKLSLTRDKRRCNLSDEIKSSKDVSSGE

SEQ ID NO: 25 - protein, 142 aa, Canis lupus
  source 1..142: note = Canis familiaris
SDELSLCSEDIVLHKNKIPESNASFRKRLTRSVADESDVNVSMIVNVSCTSACTDDEDDSDLLSSSTLTLTEEELCIKDEDDDSSIATDDEIYEECNLMSGLDYIKNELQTWIRPKFSLTKDKRRCNLSDEIKGSKDVSSSE

SEQ ID NO: 26 - protein, 142 aa, Carlito syrichta
  source 1..142: note = Tarsius syrichta
SDELSLCSEDIALHKNKIPESNASFRKRLTRSAADESDVNVSMIVNVSCTSACTDDEDDSDLLSSSTLTLTEEELCIKDEDDDSSIATDDEIYEECNLMSGLDYIKNELQTWIRPKLSLARDKKRCNLSDEIKGSRDVSSSE

SEQ ID NO: 27 - protein, 142 aa, Sus scrofa
SDELSLCSEDIVLHKNKIPESNASFRKRLTRSVADESDVNVSMIVNVSCTSACTDDEDDSDLLSSSTLTLTEEELCIKDEDDDSSIATDDEIYEECNLISGLDYIKNELQTWIRPKFSLTRDKRRCSVSDEIRGSKDVSSSE

SEQ ID NO: 28 - protein, 142 aa, Felis catus
SDELSLCSEDIVLHKNKVPESNASFRKRLTRSVADESDVNVSMIVNVSCTSACTDDEDDSDLLSSSTLTLTEEELCIKDEDDDSSIATDDEIYEECNLMSGLDYIKNELQTWIRPKISLTKDKRRCNISDEIKGSKDVSSSE

SEQ ID NO: 29 - protein, 142 aa, Cavia porcellus
SDELSLCSEDIVLHKNKIPESNASFRKRLNHSVADESDVNVSMIVNVSCTSACTDDEDDSDLLSSSTLTLTEEELCIKDEDDDSSIATDDEIYEECNLMSGLDYIKNELQTWIRPKLSLTRDKKRCNLSEEIKGNKDVSSNE

SEQ ID NO: 30 - protein, 142 aa, Bos taurus
SDELSLCSEDIVLHKNKIPESNVSFRKRLPRSVADESDVNVSMIVNVSCTSACTDDEDDSDLLSSSTLTLTEEELCVKDEDDDSSIATDDEIYEDCNVMSGLDYIKNELQTWIRPKLSLTRDKRRCNLSDEIKGNKDVSSSE

SEQ ID NO: 31 - protein, 142 aa, Sorex araneus
SDEISLCSEDIALHKNKVPESNASFRKRLTHSVADESDVNVSMIVNVSCTSACTDDEDDSDLLSSSTLTLTEEELCIKDEDDDSSIATDDEIYEDCHLMSGLDYIKNELQTWIRPKYSMTRDKKRCHLGDEIKGSKDISGSE

SEQ ID NO: 32 - protein, 142 aa, Dipodomys ordii
SDELSLCSEDIVLHKNKIPESNSSFRKRLNRSVAEESDVNVSMIVNVSCTSACTDDEDDSDLLSSSTLTLTEEELCIKDEDDDSSIATDDEIYEECNLMSGLDYIKNELQTWIRPKLFMTRDKKRCNFSNNMKGSKDGSSTE

SEQ ID NO: 33 - protein, 145 aa, synthetic construct
SDELSLCSEDIVLHKNKIPESNASFRKRMTHSVADESDINVSMIVNVSCTSACTDDEDDSDLLSSSTLTLTEEELCIKDGDDDDDDSSIATDDEIYEECNLVSGLDYIKNELQTWIRPKFSLTRDKKRCNVSDEIKGNKDVSSSE

SEQ ID NO: 34 - protein, 142 aa, Rattus sp.
SDELSLCSEDIVLHKNKIPESNASFRKRLNRSVADESDVNVSMIVNVSCTSACTDDEDDSDLLSSSTLTLTEEELCLKDEDDDSSIATDDEIYEESNLMSGLDYIKNELQTWIRPKLSLTREKKRSGVTDEIKVNKDGGGNE

SEQ ID NO: 35 - protein, 142 aa, Mus musculus
SDELSLCSEDIVLHKNKVPESNASFRKRLNRSVADESDVNVSMIVNVSCTSACTDDEDDSDLLSSSTLTLTEEELCLKDEDDDSSIATDDEIYEESNLMSGLDYIKNELQTWIRPKLSLSREKKRPSVTDEIKVNKDGGSTE

SEQ ID NO: 36 - protein, 143 aa, Monodelphis domestica
SDELSLCSEDIVLHRNKIPQDSNASFRKRLTHSVADESDVNVSMIVNVSCTSACTDDEDDSDLLSSSTLTLTEEELCVKDEDDDSSIATDEDIYEECNLMSGLDYIKNELQTWIRPKFSLSREKRRCNLSDEIQCSKDRGSSE

SEQ ID NO: 37 - protein, 142 aa, Macropus eugenii
SDELSLCSEDIILHRNKIPQDSNASFRKRLTHSVADESDVNVSMIVNVSCTSACTDDEDDSDLLSSSTLTLTEEELCVRDEDESSIATDEEIYEECNLMSGLDYIKNELQTWIRPKFSLSREKRRCNLSDEIQCSKDRGGSE

SEQ ID NO: 38 - protein, 141 aa, Taeniopygia guttata
SEDLSLCSEDIVLHRNKIPDSNASFRKHLNRSVADESDVNVSMIVNVSCTSACTDDEDDSDLLSSSTLTLTEEELGIKDEDDDSSIATDEDIYEDCNLISGLDYIKNELQTWIRPKFSLTREKRKSNLSDEIQRSKEVSND

SEQ ID NO: 39 - protein, 140 aa, Gallus gallus
SEDLSLCSEDIVLHRNKIPDSNASFRKHLNRSVADESDVNVSMIVNVSCTSACTDDEDDSDLLSSSTLTLTEEELGIKDEDDDSSIATDEDIYEECNLISGLDYIKNELQTWIRPKFSLTREKRRSNLSDEIQHGKVSNE

SEQ ID NO: 40 - protein, 134 aa, Xenopus tropicalis
SEELSLRSEDLVVLKGKLLGSNMSFHKHLSRSLGGEEGEANLSMIVNVSCTSACTDEEDDSDLLSSSTLTLTEEEEEEFEEPNSSLGSEDEAEDGTKLPLGADFIRREMQAWIRPAFFLPKEKRKINRIVKDMP

SEQ ID NO: 41 - protein, 139 aa, Pimephales promelas
TEDLSVASEDLAVQMNRCTAADSNASFRKHCRSQQQTDETDASISMVVNVSCTSACTDDEEDSDLLSSSTLTLTEEELGIKDDDSSVTSEEEYIEGSFTLGLDYIKDEFQSWIKPRSQSREKNEADLVDELQCGTLSRD

SEQ ID NO: 42 - protein, 139 aa, Danio rerio
TEDSSVASEDLAVQMNRCIAAESNASFRKHCHSQQQADETDASISMVVNISCTSACTDDEEDSDLLSSSTLTLTEEELGIKDDDSSVTSEEEYIEGSFALGLDYIKGEFQSWMKPRSQIREKNEADLVDELQCGTLSRD

SEQ ID NO: 43 - protein, 149 aa, synthetic construct
  VARIANT 1..4: note = X is any amino acid
  VARIANT 6..7: note = X is any amino acid
  VARIANT 11..17: note = X is any amino acid
  VARIANT 23: note = X is any amino acid
  VARIANT 25: note = X is any amino acid
  VARIANT 30..37: note = X is any amino acid
  VARIANT 40..44: note = X is any amino acid
  VARIANT 50: note = X is any amino acid
  VARIANT 47: note = X is any amino acid
  VARIANT 59: note = X is any amino acid
  VARIANT 61: note = X is any amino acid
  VARIANT 78..83: note = X is any amino acid
  VARIANT 84..86: note = X is any amino acid or absent
  VARIANT 18: note = X is any amino acid or absent
  VARIANT 19..21: note = X is any amino acid
  VARIANT 22: note = X is any amino acid or absent
  VARIANT 28: note = X is any amino acid
  VARIANT 38: note = X is any amino acid or absent
  VARIANT 87..89: note = X is any amino acid
  VARIANT 92..106: note = X is any amino acid
  VARIANT 108: note = X is any amino acid
  VARIANT 110: note = X is any amino acid
  VARIANT 112..113: note = X is any amino acid
  VARIANT 115..117: note = X is any amino acid
  VARIANT 119..120: note = X is any amino acid
  VARIANT 122..128: note = X is any amino acid
  VARIANT 130..133: note = X is any amino acid
  VARIANT 134: note = X is any amino acid or absent
  VARIANT 135..148: note = X is any amino acid
  VARIANT 149: note = X is Glu or absent
XXXXSXXSEDXXXXXXXXXXXXSNXSFXKXXXXXXXXXEXXXXXSMXVNXSCTSACTDXEXDSDLLSSSTLTLTEEEXXXXXXXXXXXXSSXXXXXXXXXXXXXXXGXDXIXXEXXXWXXPXXXXXXXKXXXXXXXXXXXXXXXXXXXE

SEQ ID NO: 44 - protein, 149 aa, synthetic construct; identical to SEQ ID NO: 43
  VARIANT 1: note = X is Ser or Thr
  VARIANT 2: note = X is Asp or Glu
  VARIANT 3: note = X is Glu or Asp
  VARIANT 4: note = X is Leu, Ile, or Ser
  VARIANT 6: note = X is Leu or Val
  VARIANT 7: note = X is Cys, Arg, or Ala
  VARIANT 11: note = X is Ile or Leu
  VARIANT 12: note = X is Val, Ile, or Ala
  VARIANT 13: note = X is Leu or Val
  VARIANT 14: note = X is His, Leu, or Gln
  VARIANT 15: note = X is Lys, Arg, or Met
  VARIANT 16: note = X is Asn or Glu
  VARIANT 17: note = X is Lys or Arg
  VARIANT 18: note = X is Cys or Absent
  VARIANT 19: note = X is Ile, Val, Leu, or Thr
  VARIANT 20: note = X is Pro, Leu, or Ala
  VARIANT 21: note = X is Glu, Gln, Asp, Gly, or Ala
  VARIANT 22: note = X is Asp, Glu, or absent
  VARIANT 25: note = X is Ala, Val, Ser, or Met
  VARIANT 28: note = X is Arg or His
  VARIANT 30: note = X is Arg or His
  VARIANT 31: note = X is Leu, Met, or Cys
  VARIANT 32: note = X is Thr, Pro, Asn, Arg, or His
  VARIANT 33: note = X is Arg, His, or Ser
  VARIANT 34: note = X is Ser or Gln
  VARIANT 35: note = X is Val, Ala, Leu, or Gln
  VARIANT 36: note = X is Ala, Gly, or Gln
  VARIANT 37: note = X is Asp, Glu, Gly, Thr, or Ala
  VARIANT 38: note = X is Glu, Asp, or absent
  VARIANT 40: note = X is Ser, Gly, or Thr
  VARIANT 41: note = X is Asp or Glu
  VARIANT 42: note = X is Val, Ile, or Ala
  VARIANT 43: note = X is Asn or Ser
  VARIANT 44: note = X is Val, Leu, or Ile
  VARIANT 47: note = X is Ile or Val
  VARIANT 50: note = X is Val or Ile
  VARIANT 59: note = X is Asp or Glu
  VARIANT 61: note = X is Asp or Glu
  VARIANT 78: note = X is Leu or Glu
  VARIANT 79: note = X is Cys, Gly, or Glu
  VARIANT 80: note = X is Ile, Leu, Val, or Glu
  VARIANT 81: note = X is Lys, Arg, or Phe
  VARIANT 82: note = X is Asp or Glu
  VARIANT 83: note = X is Glu, Gly, or Asp, or absent
  VARIANT 84..86: note = X is Asp or absent
  VARIANT 89: note = X is Asp, Glu, or Asn
  VARIANT 92: note = X is Ile, Leu, or Val
  VARIANT 93: note = X is Ala, Gly, or absent
  VARIANT 94: note = X is Thr or Ser
  VARIANT 95: note = X is Asp, Glu, or Ser
  VARIANT 96: note = X is Asp or Glu
  VARIANT 97: note = X is Asp or Glu
  VARIANT 98: note = X is Ile, Ala, or Glu
  VARIANT 99: note = X is Tyr or Glu
  VARIANT 100: note = X is Glu, Asp, or Ile
  VARIANT 101: note = X is Asp, Glu, or Gly
  VARIANT 102: note = X is Cys, Ser, Thr, or Gly
  VARIANT 103: note = X is Asn, Thr, His, Lys, or Ser
  VARIANT 104: note = X is Leu, Val, or Phe
  VARIANT 105: note = X is Met, Ile, Val, Pro, Thr, or Ala
  VARIANT 106: note = X is Ser or Leu
  VARIANT 108: note = X is Leu or Ala
  VARIANT 110: note = X is Tyr or Phe
  VARIANT 112: note = X is Lys or Arg
  VARIANT 113: note = X is Asn, Arg, Asp, or Gly
  VARIANT 115: note = X is Leu, Met, or Phe
  VARIANT 116: note = X is Gln or His
  VARIANT 117: note = X is Thr, Ala, or Ser
  VARIANT 119: note = X is Ile or Met
  VARIANT 120: note = X is Arg or Lys
  VARIANT 123: note = X is Leu, Phe, Tyr, Ile, or absent
  VARIANT 122: note = X is Lys, Ala, or Arg
  VARIANT 124: note = X is Ser or Phe
  VARIANT 126: note = X is Thr, Val, Ser, Ala, Pro, or Ile
  VARIANT 125: note = X is Leu, Met, or Gln
  VARIANT 127: note = X is Arg or Lys
  VARIANT 128: note = X is Asp or Glu
  VARIANT 130: note = X is Lys, Arg, or Asn
  VARIANT 131: note = X is Arg, Lys, or Glu
  VARIANT 132: note = X is Cys, Ser, Pro, Ile, or Ala
  VARIANT 133: note = X is Asn, Ser, His, Gly, or Asp
  VARIANT 134: note = X is Arg or absent
  VARIANT 135: note = X is Val, Leu, Ile, or Phe
  VARIANT 136: note = X is Ser, Gly, Thr, or Val
  VARIANT 137: note = X is Asp, Gly, Asn, or Lys
  VARIANT 138: note = X is Glu, Asn, or Asp
  VARIANT 139: note = X is Met, Ile, or Leu
  VARIANT 140: note = X is Lys, Arg, Gln, or Pro
  VARIANT 141: note = X is Gly, Val, Ser, Cys, Arg, His, or absent
  VARIANT 142: note = X is Ser, Cys, Asn, Gly, or absent
  VARIANT 143: note = X is Lys, Arg, Thr, or absent
  VARIANT 144: note = X is Asp, Val, Glu, Leu, or absent
  VARIANT 145: note = X is Ile, Val, Gly, Arg, Ser, or Absent
  VARIANT 146: note = X is Ser, Gly, Asn, Arg, or absent
  VARIANT 147: note = X is Ser, Gly, Asn, Glu, Asp, or Absent
  VARIANT 148: note = X is Ser, Gly, Asn, Thr, Asp, or absent
  VARIANT 149: note = X is Glu or absent

SEQ ID NO: 45 - protein, 134 aa, Homo sapiens
EEKSSTPLPLDTTDSGLDDKEDIECFFEACVEGDSDGEEPCFSSAPPNESAVPSEAAMPLQATACSSEFSDSSLSADDADTVALSSPSSQERAEVGKEVNGLPQTSSGCAENLEFTPSKLDSEKESSGKPGESG

SEQ ID NO: 46 - protein, 134 aa, Pan paniscus
EEKSSTPLPLDTTDSGLDDKEDIECFFEACVEGDSDGEEPCFSSAPPNESAVPSEAAMPLQATACSSEFSDSSLSADDADTVALSSPSSQERAEVGKEVNGLPQTSNGCADNLEFTLSKLDSEKESSGKPGESG

SEQ ID NO: 47 - protein, 134 aa, Macaca mulatta
EEKSSTPLPLDPTDSGLDDKEDIECFFEACVEGDSDGEEPCFSSAPPNESAVPSKAAVPLQVTACSSEFGDSSLVADDADTVALSSPSSQERAEAGKEVNGLPQTSNGSAENLEFTPSKLDSEKESSEKPGESG

SEQ ID NO: 48 - protein, 135 aa, Carlito syrichta
  source 1..135: note = Tarsius syrichta
EEKSNTPLPLDTTDSGQDDKEDIECFFEACVEDGSDGEEEACFSSSLPNESAVRNEAAMLLQETASSSKLSDISLLADDADMMALSSPTPQKGVDVGKEVDGLPQTSSGCAEISEFIPSRLGNKKESSGKPSESG

SEQ ID NO: 49 - protein, 134 aa, Equus asinus
EEKSSTPLPLDVTDSGLEDKEDIECFFEACVEDDPDGDEPCFSSVPPNEPAVPGKAAMPLPAAGSSHFSDMSLSAHDADSVALSHPPPEKGSDSGKEVDGLPQASNVCGESSELTTPSRLGSEKESSENRGESG

SEQ ID NO: 50 - protein, 134 aa, Choloepus hoffmanni
EEKSNTPLPLDTTDSGLDDREDIECFFEACVDDDPDGEEPCFSSTPPSEPAIPNEAAMPLQATSSARFSGISLLAIDAGMVAFPSPPLQKVYDAGKEVHGLPQTSNGCLESSELTTPSRPSSEKESSENPSESG

SEQ ID NO: 51 - protein, 135 aa, Phyllostomus hastatus
EEKSNTPLPLDITDSGLEDKEDIECFFEACVEDDPEEDEPRFSSASLNEPAGPSEAAMPIQAAAGSPRSSDIFLLTDDADMVALSNPPPPEGSDIGKEVDAVPQSSNVCGESSESTNPSRLASEKESSEHPGESG

SEQ ID NO: 52 - protein, 134 aa, Cavia porcellus
EEKSNTPLLLDTIDSGLDEKEDIECFFEACVENDPNSEDPCFSNTPPDESSVPEETKVPLQATPHSSKFSDTSLLVNDTDMVPLPIPPSQKEADIRKEVDDFPQTSSRYAENSEFIPSQVHSEKENSGKPGESG

SEQ ID NO: 53 - protein, 133 aa, Dasypus novemcinctus
EEKSNTPLPPDTTDPALDDREDIECFFEACVEDDPDGEEPSFSNAPPNEPMVVPDEAVLPLQATRSSLKFNDISLLANDAVTMAFSSPVPQKGYDIGKEVAGLPQTSNGCLESSELTTPSSEKESSENPGESG

SEQ ID NO: 54 - protein, 135 aa, Sus scrofa
EEKSNTPLPPDMTDSGSEDKEDTECFFEACVEDNPDEDKPCFSSAPPNETAVPGEAPGPLQAAAGSSEFSNTPLLADHADIVALSSPPLPQGGGVGQEVDGAPQASNVCGKSSELTTPSRIGSEKESSENPGESG

SEQ ID NO: 55 - protein, 135 aa, Tursiops truncatus
EEKSNTPLPPDMTDSGSEDKEDTECFFEACVEEDPSEYQSCFSSALPNEPAVPSEAPVPLQAAAGSSKFSDTSLLVDDADVMALSGPPPPKRGDVGKEVDGLPQASNVYGESSELTTPSRLSGEKESSESSGESG

SEQ ID NO: 56 - protein, 134 aa, Dipodomys ordii
DEKSNTPLPLDATDSGLDDKEDIEYFFEACVEDEPNGEEPCFSSASPKEAAVPNEATGSAQAMASSSMFNDISLLADDAHVVPLSRPPPQEEADIRREVDDWSQSSQGCAEYSIITPSGVGSEKESSGNPDVSG

SEQ ID NO: 57 - protein, 132 aa, Felis catus
EEKSNTPLPLDISDSGLEDKEDTECFFEACVEDDPDGVEPCLPHAPPNEPVSVEAALPQQAASGSSKFKDISLVADEEDMVALSGSPHQKESDVGKEGGGLPRATVHGKSSELTTSRLGSEKESLENTGESG

SEQ ID NO: 58 - protein, 133 aa, Bos taurus
EEKSSTPLPTDMTDSGSEDKEDTEYFFEACVEEDPAEDKPCFSDVPPNEPADPTEAAVPLLAGAGSSRFSDTSLLASNADMVALSSPPKGGDVGKEVDSLPEASNVHGESAEVTTPSRLGSEKESSENPGESA

SEQ ID NO: 59 - protein, 133 aa, Canis lupus
  source 1..133: note = Canis lupus familiaris
EEKSNTPVPPDITDSGLEDKEDPECFFEACVEDDPDGDGPCLSSTPPDEPTLSDEAATPPQAAAGSPTFSDTSLLADEVDMVALSNPPHQKESDVGKEADGLPPAPICGESSELTASRLGSKRESSENTGESG

SEQ ID NO: 60 - protein, 136 aa, Erinaceus europaeus
EEDSNTPLPLDITDSGLEDKEDIECFFEACIEDNPEGDEPCLSSTPPDEPSVPGEAAMTLFKAAADCFKSSDIALLSDDADTVALSSPSLQKEPVIGKEVDGLSQASSVSGENSELTTLSKIGSEKEHSKNLGDLG

SEQ ID NO: 61 - protein, 118 aa, Loxodonta africana
EEKSNTPLPQDTADSGLDDKEDIECFFEACVEDDPDGEEPSLASPSPNEPTVPDEPAVSLQAQDADMVALPSPPSEKGSDVEKEVDGPPQTSSGCEEISALATPSSEEESPGNPGESG

SEQ ID NO: 62 - protein, 135 aa, Sorex araneus
EEKSSTPLPPDAADSSLEDKEDIECFFEACVEDGPDVREPCFPSSPPNTPAVPAEASVPLQAIAGPSHSGDGQHPADDALGVTYSSLPPPNGSDIVKEVGVLPQAPSDSRENPELTESLKLGNDKESSEDLGESG

SEQ ID NO: 63 - protein, 129 aa, Mus musculus
DEKSNTPLPPDTVDSGLDDKEDVDCFFEACVEDEPADEEARLSSALPNESEVQDEAAKPEQMTASSSVFRDETDTVPLSGLSPQKGADDAKEGDGASHTSQGCVESAEPTTPPGKAKREGSSRKQSVSG

SEQ ID NO: 64 - protein, 130 aa, Rattus sp.
DEKSSTPLPPDAVDSGLDDKEDMDCFFEACVEDEPVNEEAGLPGALPNESAIEDGAEQKSEQKTASSPVLSDKTDLVPLSGLSPQKGADDAKEGDDVSHTSQGCAESTEPTTPSGKANAEGRSRMQGVSA

SEQ ID NO: 65 - protein, 138 aa, synthetic construct
  VARIANT 1..3: note = X is any amino acid
  VARIANT 5: note = X is any amino acid
  VARIANT 8..10: note = X is any amino acid
  VARIANT 12..13: note = X is any amino acid
  VARIANT 15..20: note = X is any amino acid
  VARIANT 22..25: note = X is any amino acid
  VARIANT 31..39: note = X is any amino acid
  VARIANT 101..119: note = X is any amino acid
  VARIANT 40: note = X is any amino acid or absent
  VARIANT 41..51: note = X is any amino acid
  VARIANT 52: note = X is any amino acid or absent
  VARIANT 53..60: note = X is any amino acid
  VARIANT 61: note = X is any amino acid or absent
  VARIANT 62..100: note = X is any amino acid
  VARIANT 120: note = X is any amino acid or absent
  VARIANT 121..138: note = X is any amino acid
XXXSXTPXXXDXXDXXXXXXEXXXXFFEACXXXXXXXXXXXXXXXXXXXXXXXXXXXXXXXXXXXXXXXXXXXXXXXXXXXXXXXXXXXXXXXXXXXXXXEXXXXXXXXXXXXXXXXXXXXXXXXXXXXXXXXXXXXX

SEQ ID NO: 66 - protein, 138 aa, synthetic construct; identical to SEQ ID NO: 65
  VARIANT 1: note = X is Met, Glu, or Asp
  VARIANT 2: note = X is Gly or Glu
  VARIANT 3: note = X is Lys or Asp
  VARIANT 5: note = X is Ser or Asn
  VARIANT 8: note = X is Leu or Val
  VARIANT 9: note = X is Pro or Leu
  VARIANT 10: note = X is Leu, Pro, Thr, or Gln
  VARIANT 12: note = X is Thr, Pro, Val, Ile, Met, or Ala
  VARIANT 13: note = X is Thr, Ile, Ser, Ala, or Val
  VARIANT 15: note = X is Ser or Pro
  VARIANT 16: note = X is Gly, Ala, or Ser
  VARIANT 17: note = X is Leu, Gln or Ser
  VARIANT 18: note = X is Asp or Glu
  VARIANT 19: note = X is Asp or Glu
  VARIANT 20: note = X is Lys or Arg
  VARIANT 22: note = X is Asp or Glu
  VARIANT 23: note = X is Ile, Thr, Pro, Val, or Met
  VARIANT 24: note = X is Glu or Asp
  VARIANT 25: note = X is Cys or Tyr
  VARIANT 31: note = X is Val or Ile
  VARIANT 32: note = X is Glu or Asp
  VARIANT 33: note = X is Gly, Asp, Asn, or Glu
  VARIANT 34: note = X is Asp, Gly, Asn, or Glu
  VARIANT 35: note = X is Pro or Ser
  VARIANT 36: note = X is Asp, Ser, Asn, Ala or Val
  VARIANT 37: note = X is Gly, Glu, Ser, Val, Asp, or Asn
  VARIANT 38: note = X is Glu, Asp, Tyr, Val, or Arg
  VARIANT 39: note = X is Glu, Asp, Lys, Gln, or Gly
  VARIANT 40: note = X is Glu or absent
  VARIANT 41: note = X is Pro, Ala, or Ser
  VARIANT 42: note = X is Cys, Arg, Ser, or Gly
  VARIANT 43: note = X is Phe or Leu
  VARIANT 44: note = X is Ser or Pro
  VARIANT 45: note = X is Ser, Asn, His, Asp, or Gly
  VARIANT 46: note = X is Ala, Val, Ser, Pro or Thr
  VARIANT 47: note = X is Pro, Leu, or Ser
  VARIANT 48: note = X is Pro or Leu
  VARIANT 49: note = X is Asn, Ser, Asp, or Lys
  VARIANT 50: note = X is Glu or Thr
  VARIANT 51: note = X is Ser, Pro, or Thr
  VARIANT 52: note = X is Met or absent
  VARIANT 53: note = X is Ala, Ser, Val, Thr, Glu, or absent
  VARIANT 54: note = X is Val, Gly, Asp, Ile, or Leu
  VARIANT 55: note = X is Pro, Arg, Ser, Gln, or Glu
  VARIANT 56: note = X is Ser, Asn, Gly, Asp, Val, or Thr
  VARIANT 57: note = X is Glu or Gly
  VARIANT 58: note = X is Ala, Thr, or Pro
  VARIANT 59: note = X is Ala, Lys, Val, Pro, Thr, Ala, Ser or Glu
  VARIANT 60: note = X is Met, Val, Leu, Gly. Thr, Lys, or Gln
  VARIANT 61: note = X is Thr, Lys, or absent
  VARIANT 62: note = X is Pro, Leu, Ser, or absent
  VARIANT 63: note = X is Leu, Pro, Gln, Ile, Ala, Phe, Glu, or absent
  VARIANT 64: note = X is Gln, Leu, Lys, or absent
  VARIANT 65: note = X is Ala, Val, Glu, Pro, Met, Lys, or absent
  VARIANT 66: note = X is Thr, Ala, Met, Gly, or Ile, absent
  VARIANT 67: note = X is Ala, Pro, Arg, Ser, or absent
  VARIANT 68: note = X is Cys, Ser, Gly, His, Asp, or absent
  VARIANT 69: note = X is Ser, Cys, Pro, or absent
  VARIANT 71: note = X is Glu, Lys, Met, His, Arg, Thr, or absent
  VARIANT 70: note = X is Ser, Ala, Pro, Leu, Phe, or absent
  VARIANT 72: note = X is Ser, Leu, Phe, or absent
  VARIANT 73: note = X is Ser, Gly, Asn, Lys, or absent
  VARIANT 74: note = X is Asp, Gly, Asn, or absent
  VARIANT 75: note = X is Ser, Ile, Met, Thr, Gly, or absent
  VARIANT 76: note = X is Ser, Phe, Pro, Ala, Gln, or absent
  VARIANT 77: note = X is Leu, His, or Val
  VARIANT 78: note = X is Ser, Val, Leu, Gln, Pro, or Phe
  VARIANT 79: note = X is Ala, Thr, Val, Arg, or Ser
  VARIANT 80: note = X is Asp, Asn, Ile, His, Gln, or Ser
  VARIANT 81: note = X is Asp, His, Glu, Asn, or Lys
  VARIANT 82: note = X is Ala, Thr, Glu, or Val
  VARIANT 83: note = X is Asp, Gly, Val, His, or Leu
  VARIANT 84: note = X is Thr, Met, Ser, Ile, Val, Gly, or Leu
  VARIANT 85: note = X is Val or Met
  VARIANT 86: note = X is Ala, Pro, or Thr
  VARIANT 87: note = X is Leu, Phe, or Tyr
  VARIANT 88: note = X is Ser or Pro
  VARIANT 89: note = X is Ser, His, Ile, Asn, Gly, or Arg
  VARIANT 90: note = X is Pro, Ser, or Leu
  VARIANT 91: note = X is Ser, Thr, Pro, or Val
  VARIANT 92: note = X is Ser, Pro, Leu, His, or Lys
  VARIANT 93: note = X is Gln, Glu, Pro, or absent
  VARIANT 94: note = X is Glu, Lys, Gln,Asn, or absent
  VARIANT 95: note = X is Arg, Gly, Glu, or Val
  VARIANT 96: note = X is Ala, Val, Ser, Tyr, Gly, or Pro
  VARIANT 97: note = X is Glu, Asp, Val, or Gly
  VARIANT 98: note = X is Val, Ala, Ser, Ile, or Asp
  VARIANT 99: note = X is Gly, Arg, Glu, Val, or Ala
  VARIANT 100: note = X is Lys, Gln, or Arg
  VARIANT 102: note = X is Val, Gly, or Ala
  VARIANT 103: note = X is Asn, Asp, His, Ala, or Gly
  VARIANT 104: note = X is Gly, Ala, Val, Glu, Asp, or Ser
  VARIANT 106: note = X is Pro or Ser
  VARIANT 105: note = X is Leu, Val, Phe, Ala, Trp, or Pro
  VARIANT 107: note = X is Gln, Arg, Glu, Pro, His, or absent
  VARIANT 108: note = X is Thr, Ala, or Ser
  VARIANT 109: note = X is Ser, Thr, or Pro
  VARIANT 110: note = X is Ser, Asn, Gln or absent
  VARIANT 111: note = X is Gly, Val, Arg, Ile, or Asp
  VARIANT 112: note = X is Cys, Tyr, His, or Ser
  VARIANT 113: note = X is Ala, Gly, Leu, Glu, Arg, or Val
  VARIANT 114: note = X is Glu, Asp, or Lys
  VARIANT 115: note = X is Asn, Ile, Ser, or Tyr
  VARIANT 116: note = X is Leu, Ser, Pro, Ala, or Thr
  VARIANT 117: note = X is Glu, Ile, or Ala
  VARIANT 118: note = X is Phe, Leu, Ser, Ile, Val, or Pro
  VARIANT 119: note = X is Thr, Ile, or Ala
  VARIANT 120: note = X is Thr, Asn, Glu, or absent
  VARIANT 121: note = X is Pro, Leu, Thr, Ala, or Ser
  VARIANT 122: note = X is Ser or Pro
  VARIANT 123: note = X is Lys, Arg, Gln, or absent
  VARIANT 124: note = X is Leu, Pro, Val, Ile, Lys, or absent
  VARIANT 125: note = X is Asp, Gly, Ser, His, Ala, or absent
  VARIANT 126: note = X is Ser, Asn, Gly or Lys
  VARIANT 127: note = X is Glu, Lys, Asp, Arg, or Ala
  VARIANT 128: note = X is Lys, Arg, or Glu
  VARIANT 129: note = X is Glu or Gly
  VARIANT 130: note = X is Ser, Asn, His, or Arg
  VARIANT 131: note = X is Ser, Leu, or Pro
  VARIANT 132: note = X is Gly, Glu, Lys, or Arg
  VARIANT 133: note = X is Lys, His, Asn, Ser, Asp, or Met
  VARIANT 134: note = X is Pro, Arg, Ser, Thr, Leu, or Gln
  VARIANT 135: note = X is Gly, Ser, or Asp
  VARIANT 136: note = X is Glu, Val, or Asp
  VARIANT 137: note = X is Ser or Leu
  VARIANT 138: note = X is Gly or Ala

SEQ ID NO: 67 - DNA, 5887 bp, synthetic construct
  source 1..5887: mol_type = other DNA
accgagttgctctcacccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttcaattcaactactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctactcgagcctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcagtagaaaaacagccaagctagggaggctgggaggccaagccccagataccttacatagctctgctcagcctctgtctcattaggaactccatttttaggatgcagttgtttcaggctaaaaataaatcatgcaatgaataaaaaagttagatacgacactgtagagggattcgctgatacagtctgtccgatctagagcagtctgggctttcacaagacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacctaagcttgctagcgctaccggtcgccaccatgggtaaaagcagcactccattgccactagacaccactgactcgggcttagatgacaaggaagatattgaatgcttttttgaggcctgtgttgagggtgactctgatggagaggagccttgtttctctagtgctcctccaaatgaatctgcagttcccagcgaagctgcaatgccactacaagcaacagcatgttcttctgagttcagtgatagttctctttcagctgatgatgcagatacagtggctctttcaagtccttcctctcaggaaagagctgaggttggaaaggaagtgaatggtttgccccaaacttccagtggctgtgcagaaaacttagagtttactccttcaaagcttgacagtgaaaaggaaagttccggaaaaccaggtgaatctggaatgccagaagaacataatgctgcttcagccaaatctaaagttcaagacctctccttgaaggcaaatcagccaacagacaaggccgcattgcatcccagccccaaaactttaacctgtgaagaaaatcttctaaaccttcatgaaaaacgacatagaaatatgcataggtagagtgtaatgcccccacgcatggaaatcatctcattgaaagatagcctggctgaagctcagggctagttaagtttgatccgcggccgcaatcaacctctggattacaaaatttgtgaaagattgactgatattcttaactatgttgctccttttacgctgtgtggatatgctgctttaatacctctgtatcgtgctattgcttcccgtacggctttcgttttctcctccttgtataaatcctggttgctgtctctttataaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgcggccttctgccggttctgcggcctctcccgcgtcttcgctttcggcctccgacgagtcggatctccctttgggccgcctccccgcctgtaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattaccactccctctctgcgcgctcgctcgctcactgaggccgggcgaccaaaggtcgcccgacgcccgggctttgcccgggcggcctcagtgagcgagcgagcgcgccagctgaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaatcagccagccggaagggccgagcgcagaagtggtcctgcaactttatccgcctccatccagtctattcattgttgccgggaagctagagtaagtagttcgccagttaatagttttcacaacgttgttgccattgctacaggcatcgtggtgtcacgctcgtcgtttggtatggcttcattcagctccggttcccaacgatcaaggcgagttacatgtcaccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcg

SEQ ID NO: 68 - protein, 190 aa, synthetic construct; identical to SEQ ID NO: 14

SEQ ID NO: 69 - RNA, 11191 nt, synthetic construct
  source 1..11191: mol_type = other RNA
ctgttttggaaagaagtgaggtttagacttctccatgttaaccatgagcgtgacactttcccccctgaggtcacaggacctggatcccatggctactgatgcttcacccatggccatcaacatgacacccactgtggagcagggtgagggagaagaggcaatgaaggacatggactctgaccagcagtatgaaaagccacccccactacacacaggggctgactggaagattgtcctccacttacctgaaattgagacctggctccggatgacctcagagagggtccgagacctaacctattcagtccagcaggattcggacagcaagcatgtggatgtacatctagttcaactaaaggacatttgtgaagatatttctgatcatgttgagcaaatccatgccctccttgaaacagagttctccctaaagctgctgtcttactctgtcaacgtgatagtggacatccacgcagtgcagctcctctggcaccagcttcgagtctcagtgctggttctgcgggagcgcattctgcaaggtctgcaggacgccaatggcaactacactaggcagacggacattctgcaagctttctctgaagagacaaaagagggccggcttgattctctaacagaagtggatgactcaggacaattaaccatcaaatgttctcaaaattacttgtctctggattgtggcattactgcattcgaactgtctgactacagtccaagtgaggatttgctcagtgggctaggtgacatgacctctagccaagtcaaaaccaaaccctttgactcttggagctacagtgagatggaaaaggagtttcctgagcttatccgaagtgttggtttacttacggtagctgctgactctatctctaccaatggcagtgaagcagttactgaggaggtatctcaagtatctctctcagtagacgacaaaggtggatgtgaggaagacaatgcttctgcagtcgaagagcaaccaggcttaacactgggggtgtcatcatcttcaggagaagctctgacaaatgctgctcaaccctcctctgagactgtgcagcaagaatccagttcctcctcccatcatgatgcaaagaatcagcagcctgttccttgtgaaaatgcaacccccaaacgaaccatcagagattgctttaattataacgaggactctcccacgcagcctacattgccaaaaagaggactttttcttaaagaggaaacttttaagaatgatctgaaaggcaatggtggaaagaggcaaatggttgatctaaagcctgagatgagcagaagcaccccttcgctagtagatcctcctgacagatccaaactttgcctggtattgcagtcttcttaccccaacagcccttctgctgccagccagtcttatgagtgtttacacaaggtggggaatgggaaccttgaaaacacagtcaaatttcacattaaagaaatttcttccagcctgggaaggcttaacgactgctataaagagaaatctcgacttaaaaagccacacaagacctcagaagaggtgcctccatgccgaacacctaaacgggggactggttcaggcaaacaagctaaaaatacaaagagctcagcagtgccaaatggagagctttcttatacttccaaggccatagaggggccacaaacaaattctgcttccacatcctcacttgagccttgtaatcagagaagttggaatgccaaattgcaattgcagtcagaaacatccagttcaccagcttttactcagagcagtgaatcctctgttggctcagacaacatcatgtctccggtgccacttctttcaaaacacaaaagcaaaaaaggtcaagcctcctctccaagtcacgtcactaggaatggtgaggttgtggaggcctggtatggctctgatgaatacctagcactgccctctcaccttaagcagacagaagtattggctttgaagttggaaaacctaacaaagcttctgcctcagaaacccagaggagaaaccatccagaatattgatgactgggaactgtctgaaatgaattcagattctgaaatctatccaacctatcatgtcaaaaagaagcatacaaggctaggcagggtgtctccaagctcatctagtgacatagcctcttcactaggggagagcattgaatctgggcccctgagtgacattctttctgatgaggagtccagtatgcctctcgctggcatgaaaaagtatgctgatgagaagtcagaaagagcttcatcctctgagaaaaatgagagccattctgccactaaatcagctttaattcagaaactgatgcaagatattcagcaccaagacaactatgaagccatatgggaaaaaatagaggggtttgtaaacaaactggatgaattcattcaatggttaaatgaagccatggaaactacagaaaattggactccccctaaagcagagatggatgaccttaaactgtatctggagacacacttgagttttaagttgaatgtagacagtcattgtgctctcaaggaagctgtggaggaggaaggacaccaacttcttgagcttattgcatctcacaaagcagaaggactgaaggacatgctgcggatgattgcaagtcaatggaaggagctgcagaggcaaatcaaacggcagcacagctggattctcagggctctggataccatcaaagccgagatactggctactgatgtgtctgtggaggatgaggaagggactggaagccccaaggctgaggttcaactatgctacctggaagcacaaagagatgctgttgagcagatgtccctcaagctgtacagcgagcagtataccagcagcagcaagcgaaaggaagagtttgctgatatgtcaaaagttcattcagtgggaagcaatgggcttctggactttgattcagaatatcaggagctctgggattggctgattgacatggagtcccttgtgatggacagccacgacctgatgatgtcagaggagcagcagcagcatctttacaagcgatacagtgtggaaatgtccatcagacacctgaaaaagacggagctgcttagtaaggttgaagctttgaagaaaggtggcgttttactaccaaatgatctccttgaaaaagtggattcaattaatgaaaaatgggaactgcttgggaaaaccctaggagagaagatccaggacacaatggcagggcacagtgggtcgagtccacgtgacctgctctctcctgaaagtggaagcctggtaaggcagctggaggtcaggatcaaagaactgaaaggatggctaagagatacagagcttttcatcttcaattcctgtctgagacaagaaaaggaaggaacaatgaatactgagaaacaactgcaatactttaagtccctctgtcgtgaaatcaagcaacgacgtcgaggagttgcctccattctgcgactatgccagcatcttttggatgaccgggagacttgcaatctgaatgcagaccaccagcccatgcagctgatcattgtaaatcttgaaagaaggtgggaagccattgtcatgcaagccgtccagtggcaaacacgtctacaaaagaagatgggaaaggaatctgagactttgaatgtgattgatcctggcttgatggacctaaatgggatgagtgaggatgccctggaatgggatgaaatggacataagtaacaagttaattagtttgaatgaggaatcaaatgaccttgatcaagaactccaacctgttatcccttccttgaagcttggagagacaagtaatgaggaccctggttatgacgaggaggctgataaccatgggggatctcagtatgcctcaaatattactgccccctctagtccacacatttaccaggtgtacagcctccacaatgttgaactctatgaggacaaccacatgccatttctgaaaaacaatccaaaggtcactggcatgacacagcctaatgttttaactaagagtctcagtaaagactcttcattttcatctaccaaatctttgccagatcttctaggtggttccaatttggtaaagccctgcgcatgtcatggaggagacatgagccagaattcaggcagtgagagtggaattgtcagtgaaggagacacagaaaccactaccaactctgaaatgtgcttgctcaatgcagtggatgggtccccaagtaaccttgaaactgaacatctggacccacaaatgggagatgcagttaacgtgttaaagcaaaaatttacagatgagggggaaagcattaagcttccaaatagctctcagtcgtccatttcaccagtgggttgtgtaaatggaaaagttggagatttaaacagtattaccaaacatacccctgactgtttgggagaagaattacaaggaaaacatgatgtgtttacattttatgattactcatacctccaaggctcaaaactcaaattaccaatgataatgaaacagtcacaaagcgaaaaagcgcatgtggaggatcccctgcttcgtggtttttattttgataaaaaatcatgcaaatctaaacatcagactacagagttacaaccagatgtacctccccatgaaaggattttggcaagtgcatctcatgaaatggatcgcatttcatataaaagtggcaatatagaaaagacattcactggcatgcagaatgccaaacagctctcccttttatctcatagttcatctattgagtccctttctccagggggtgatttatttggattgggcatctttaaaaatggcagtgacagcctccagcgaagcacttctttagaaagttggttgacttcctataaaagcaatgaagatctctttagctgtcacagctctggggatataagcgtgagcagtggctcagttggtgaactaagtaaaagaacattagatctcctgaatcgtttggagaatatccagagcccctcagagcaaaagataaaacgaagtgtttctgatatcactcttcaaagcagttcccaaaagatgtcctttactggccagatgtcattggacatagcatcttctatcaatgaagactcagcggcatctctaacagaacttagcagcagtgacgagctctctctttgctcagaggatattgtgttacacaagaacaagatcccggaatcgaatgcatcgttcaggaagcgtctgactcgttcagtggctgatgaaagcgatgtcaatgtcagcatgattgttaatgtctcttgcacctctgcttgcactgatgatgaagatgacagcgacctgctctccagctctacccttaccttgactgaagaagagctgtgcatcaaagatgaggatgacgactccagtattgcaacagatgatgaaatttatgaagactgcaccttgatgtcagggctagactacataaagaatgaattacagacctggattaggccaaaattgtctttgacaagagataagaaaaggtgcaatgtcagtgatgagatgaagggcagtaaagatataagtagcagtgagatgaccaatccctctgatactctgaatattgagacccttctaaatggctctgtaaaacgtgtctctgaaaataatggaaatggtaagaattcatctcatacccatgagttagggacaaagcgtgaaaataagaaaactattttcaaagttaataaagatccatatgtggctgacatggaaaatggcaatattgaaggtattccagaaaggcaaaagggaaaaccgaatgtgacttcaaaggtatcagaaaatcttggttcacatgggaaagagatttcagagagtgagcattgtaagtgtaaagcacttatggatagtttagatgattcaaatactgctggcaaggaatttgtttcccaagatgttagacatcttccaaagaaatgtccaaatcaccaccattttgaaaatcaaagcactgcctctactcccactgagaagtctttctcagaactggctttagaaaccaggtttaacaacagacaagactctgatgcactgaaatcatctgatgatgcaccgagtatggctggaaaatctgctggttgttgcctagcacttgaacaaaacggaacagaggaaaatgcttctatcagcaacatttcctgttgcaactgtgagccagatgttttccatcaaaaagatgccgaagattgttcagtacacaactttgttaaggaaatcattgacatggcttcgacagccctaaaaagtaaatctcaacctgaaaacgaggtggctgctcctacttcattaactcaaatcaaggagaaagtgttggagcattctcaccggcccatccagctgagaaaaggggacttttattcgtacttatctctctcatctcatgacagtgattgtggggaggtcaccaattacatagaagagaaaagcagcactccattgccactagacaccactgactcgggcttagatgacaaggaagatattgaatgcttttttgaggcctgtgttgagggtgactctgatggagaggagccttgtttctctagtgctcctccaaatgaatctgcagttcccagcgaagctgcaatgccactacaagcaacagcatgttcttctgagttcagtgatagttctctttcagctgatgatgcagatacagtggctctttcaagtccttcctctcaggaaagagctgaggttggaaaggaagtgaatggtttgccccaaacttccagtggctgtgcagaaaacttagagtttactccttcaaagcttgacagtgaaaaggaaagttccggaaaaccaggtgaatctggaatgccagaagaacataatgctgcttcagccaaatctaaagttcaagacctctccttgaaggcaaatcagccaacagacaaggccgcattgcatcccagccccaaaactttaacctgtgaagaaaatcttctaaaccttcatgaaaaacgacatagaaatatgcataggtagaatgtaccccctccccaagcatgaaaatcatctcactgaaagatacgcctggctgcaactcaggggtggcctcatcctcccgccctgggctggcctctggttccatcacgtttgtcactgccgtttattacattgacttctcccaagatgaatcttccttccaaatgtgttttctccacacaagccttgtgatctgaatgtgtgcgctggttctctttaggtgatcgtctttgaagttcagcaaagctgcttgttctcccatggattcctgtcccaagctacctctaccaaccctctctctccagctagacttttctctttgcctcctcccttcccttccactctttaaagttctgcagttcaccaactggtagtccattaaattctcctgtctagaatgacccccccaccagtacttgaccaatttcatgtatcaatctggatttttttttaacggtataatgactgtgtttattgaaagagttttacctaaaaagccaacatttgaattggttgcagcatagagaagaaacactggtccttctttcaaaattaagcaactattaaaagcgccattttatttatttcatttaaaaaataatctatgcagcatttcaagaaacaaccatatggtgttgtatattataaactggtgacattctactattgaattatgtacaacattttcattttttatgcttcttgaggtggtaatgagaaaaaagttttttaaaaaagtgtgccttgctgtatttcttataccatttattaaaaagctgctttcacggtaaaattatgttggtttgaaaggaggaaatagcaaggttaagatgtgtgaataatttctgtatatatgtataaccaagtacaaacattgatgtataatgacagtataaaatgctttcatgtttgtgatgtctagtgatgtggaaaatataagccttaaatccattagattgcatggtaattaaaattggcataataaacacagattattgggggaaaaggaaaattagtgatctcttctactatgttctttaccaaattgttgcatctggttctgaaaaagtatagcatgtagcagcttccaaacatattcatattgcttaagaggcttaacattacctaaactagagactagacgtaaagccttcagttttcaaaatctttctggtcactataaagatcttggaacagcaaatgattaaatgtcagttcccctaaaccaataaacatttatactagattttttatttccacttatcattaatgatttaatgttggatttcaggtaccttgtatgtcttaatttattttaaatatttattttgaatgagtttgatagaaagctagtagaaaagtacagaaaatttgactattatttatagatttcaggtatatttatatgtgtaaaagaaattgacaaagaaatatttcatctggcctttactgactcctgttaaatgcagttttaaatttatatcgtaacacctacttaagtgcctgacacagtaggtattcaataaaaatttactgaattaaaggattaaattaggtgacatggtgacatctatccctttattttgacactaaaacatggacacaactagaaagaggtacaatgcaatataaagtcacaatagataatatatatcaaatttctaaaaggtaaagaatgttgtgggttcatgcagtcacaggaatgacaatcattcaacagatagttcagaaacactttttatctgcaaggcactattctagatccagaagatgcaatgttgaacaaacagacaaagccctgccctcagaaggctgtcctgcattaggaacaagtgaacacgcaaatgacatgaagtatttgttgcagagctgaggaacagagcaaatgtagtgatagaagcgcaatgagagaagcagcagtgggtacaaggaggaagaaaaagggcttgcagagagtggaaagttagtggaatattcatgaaacttcattgcaggggtaatagaagaaaaagtaaattgggaggacttaatggaaggtcttttaaaaagttaacttggagcttctgtatgtaaaatgctaggtaataaggacactttgtacaggctgttttgcacctgattttatttatcattagtgccacgccaagatcatttagacgatgcttatctgtaattctaccactttaataactatttgtatttttatgccccttctgatcttttccatatgtatttctaaatggataaattattctaggcttcttaataggtagtaatttgttcaaaagcggttttagccagacatctagttgcagtgttcaagaggattatgggggaaagagattagagataattgtctagttagggggcagctggagaaaataagctaagtttgcaataacagagtacacaagtatagtggcccaggatgtagtgaaagaacaaatcctagagtctttgaaatttctaagggcattctagacctctgttgggatatggtattattttacatactgacacaacctaaattttctttgggtagtaactaatgtcaagtctacatcgactggtaaaacattcaaagaacaaactgacaatgatgttctacctacttgttacatgctcatggaagaccgtgcagtattgaaagtatttgttaattatctgcttagtattaacactaaatttgtagaatgactttcaggtttgttgaacaatgccttttcaggttggaagaagaaaaatagcctcaatctcccaccccatgtaggcactacctccccaattacccttagaaaatgatcacaccaactctgcctacacacttccagtgatagtggctcattgtctgttaaggcaaactgttccactgttgggcatatctctttgttagaaagttctttcttaggttgctaaaatctgcctagtaccccgctaccctgttctgtcttatggagcagcccagattatctttactccctctttctcatggcaaccctgaagataatcaaggccagttactcatcatctcccaaccactgtttcctcaactgcccttcatatgtcatggttttcagatccattccaacctgactgaatgttaacagacagaattcttcacattaaggaactgtcttcatcatcatacatgtagaaaagaatctgaacatttaagtgcgaagttttctctagaaatatattcaagatatgtttattctattattgtaaatttcaaacaataaataaataagaatccatgacttccttcagtggcccagtccagtgcctaagtcatctggaatcttctccttacatcgaatacaaacctaacctgtttccaagtagggcactccctccgcttatttattcatttattcagccattcagcaaacatttattaaatggctacctatgtgaggcactatatttggcattaggtatataaaggcaactaacacatggtatctgtctccaaaggtttacagcttttcttctgaaagtgccttttgtgtccttgaatagagtgattatgacttttgtctcaattaaatgatgacctcatttagtttctgatgaagtttctctacatttaagaaaatctagtgagcaaaacggtatacaaaaaatcatctggatctgactaccagaaaaagtaaagtggttttaattacagaaagaaaaatatttgttagatttctggtcactgaataaatatgcatcttatacaacacagaacaacctaaatttagggcttctggaaacctgagtttgatgactttgttgtgtttggttcatccggactaggtttcacccattctataattcctagaaacaaactagatgaaaatatcaatgaaatgatccaggtctgtcttacaagtcaatttgctttagcttccaaaagaagttgaaattttttagaattttaaagaaaaactcacttttcaagtgtcatataattcttatattttaaatataaatcgaaccaactaaatttaccgctatatttttttctacatattcttttttcaattttcattttgaaaaaattcagacctatgtaaaagttgagagtagaacaaagatacttggtactcatccctcaggttcatttattgttaatatttcaccatatttacttcatctctgtatacacacacacttttttgtatgattaaatcatttgaaattaaa

SEQ ID NO: 70 - DNA, 3683 bp, synthetic construct
  source 1..3683: mol_type = other DNA
actcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaactcgatcgagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagcagtctgggctttcacaagacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacctaagcttgctagcgctcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgcgggaggaaatagcaaggttatagtgaagccacagatgtataaccttgctatttcctcccttgcctactgcctcggaattcaaggggctactttaggagcaattatcttgtttactaaaactgaataccttgctatctctttgatacatttttacaaagctgaattaaaatggtataaattaaatcacttttttcaattggaagactaatgcggcagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattaccactccctctctgcgcgctcgctcgctcactgaggccgggcgaccaaaggtcgcccgacgcccgggctttgcccgggcggcctcagtgagcgagcgagcgcgccagctgaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagt

SEQ ID NO: 71 - RNA, 52 nt, synthetic construct
  source 1..52: mol_type = other RNA
aattatgttggtttgaaaggaggaaatagcaaggttaagatgtgtgaataat

SEQ ID NO: 72 - RNA, 52 nt, synthetic construct
  source 1..52: mol_type = other RNA
aattatgttggtttgaaaggaggaaatagcaaggttaagaggtgtgaataat

SEQ ID NO: 73 - RNA, 52 nt, synthetic construct
  source 1..52: mol_type = other RNA
tattatgttggtttgaaaggaggaaatagcaaggttaagatgcgtgaataat

SEQ ID NO: 74 - RNA, 52 nt, synthetic construct
  source 1..52: mol_type = other RNA
gattacactggtttgaaaggaggaaatagcaaggttaagatgcgtgcataat

SEQ ID NO: 75 - protein, 209 aa, synthetic construct
MEQKLISEEDLSPGMLTMSVTLSPLRSQTPLPPDAVDSGLDDKEDMDCFFEACVEDEPVNEEAGLPGALPNESAIEDGAEQKSEQKTASSPVLSDKTDLVPLSGLSPQKGADDAKEGDDVSHTSQGCAESTEPTTPSGKANAEGRSRMQGVSATPEENAASAKPKIQAFSLNAKQPKGKVAMRYPSPQTLTCKEKLVNFHEDRHSNMHR

SEQ ID NO: 76 - protein, 426 aa, synthetic construct
ATGGTGGAGCTCTCTCTTTGCTCAGAGGATATTGTGTTACACAAGAACAAGATCCCGGAATCGAATGCATCGTTCAGGAAGCGTCTGACTCGTTCAGTGGCTGATGAAAGCGATGTCAATGTCAGCATGATTGTTAATGTCTCTTGCACCTCTGCTTGCACTGATGATGAAGATGACAGCGACCTGCTCTCCAGCTCTACCCTTACCTTGACTGAAGAAGAGCTGTGCATCAAAGATGAGGATGACGACTCCAGTATTGCAACAGATGATGAAATTTATGAAGACTGCACCTTGATGTCAGGGCTAGACTACATAAAGAATGAATTACAGACCTGGATTAGGCCAAAATTGTCTTTGACAAGAGATAAGAAAAGGTGCAATGTCAGTGATGAGATGAAGGGCAGTAAAGATATAAGTAGCAGTGAG

SEQ ID NO: 77 - RNA, 19 nt, synthetic construct
  source 1..19: mol_type = other RNA
ggttgaagctttgaagaaa

SEQ ID NO: 78 - RNA, 19 nt, synthetic construct
  source 1..19: mol_type = other RNA
gctaagagatacagagctt

SEQ ID NO: 79 - RNA, 19 nt, synthetic construct
  source 1..19: mol_type = other RNA
ggaggaaatagcaaggtta

SEQ ID NO: 80 - protein, 36 aa, synthetic construct
CQRSTVASMMHRQETVDCLKKFNARRKLKGAILTTM

SEQ ID NO: 81 - protein, 12 aa, synthetic construct
KKRKSSSSVQMM

SEQ ID NO: 82 - protein, 14 aa, synthetic construct
EPQTTVIHNPDGNK